(19) United States
(12) Patent Application Publication
(10) Pub. No.: US 2004/0044226 A1
(43) Pub. Date: Mar. 4, 2004

(54) Estrogen receptor modulators

(76) Inventors: Frank P. DiNinno, Old Bridge, NJ (US); Seongkon Kim, Holmdel, NJ (US); Jane Y. Wu, Marlboro, NJ (US)

Correspondence Address: MERCK AND CO INC, P O BOX 2000, RAHWAY, NJ, 070650907

(21) Appl. No.: 10/399,246

(22) PCT Filed: Oct. 15, 2001
(86) PCT No.: PCT/US01/32035

(51) Int. Cl. C07D 335/06; C07D 495/02

(52) U.S. Cl. 549/23; 549/362

(57) ABSTRACT

The present invention relates to compounds and derivatives thereof, their synthesis, and their use as estrogen receptor modulators. The compounds of the instant invention are ligands for estrogen receptors and as such may be useful for treatment or prevention of a variety of conditions related to estrogen functioning including: bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restinosis, gynacomastia, vascular smooth muscle cell proliferation, obesity, incontinence, and cancer, in particular of the breast, uterus and prostate.

### BACKGROUND OF THE INVENTION

Naturally occurring and synthetic estrogens have broad therapeutic utility, including: relief of menopausal symptoms, treatment of acne, treatment of dysmenorrhea and dysfunctional uterine bleeding, treatment of osteoporosis, treatment of hirsutism, treatment of prostatic cancer, treatment of hot flashes and prevention of cardiovascular disease. Because estrogen is very therapeutically valuable, there has been great interest in discovering compounds that mimic estrogen-like behavior in estrogen responsive tissues.

For example, estrogen-like compounds would be beneficial in the treatment and prevention of bone loss. Bone loss occurs in a wide range of subjects, including women that are post-menopausal or have had a hysterectomy, patients who were or are currently being treated with corticosteroids, and patient's having gonadal dysgenesis. The current major bone diseases of public concern are osteoporosis, hypercalcemia of malignancy, osteopenia due to bone metastases, periodontal disease, hyperparathyroidism, periarticular erosions in rheumatoid arthritis, Paget's disease, immobilization-induced osteopenia, and glucocorticoid-induced osteoporosis. All of these conditions are characterized by bone loss, resulting from an imbalance between bone resorption, i.e. breakdown, and bone formation, which continues throughout life at the rate of about 14% per year on the average. However, the rate of bone turnover differs from site to site, for example, it is higher in the trabecular bone of the vertebrae and the alveolar bone in the jaws than in the cortices of the long bones. The potential for bone loss is directly related to turnover and can amount to over 5% per year in vertebrae immediately following menopause, a condition which leads to increased fracture risk.

In the U.S., there are currently about 20 million people with detectable fractures of the vertebrae due to osteoporosis. In addition, there are about 250,000 hip fractures per year attributed to osteoporosis. This clinical situation is associated with a 12% mortality rate within the first two years, while 30% of the patients require nursing home care after the fracture.

Osteoporosis affects approximately 20 to 25 million post-menopausal women in the U.S. alone. It has been theorized that the rapid loss of bone mass in these women is due to the cessation of estrogen production of the ovaries. Since studies have shown that estrogen slows the reduction of bone mass due to osteoporosis, estrogen replacement therapy is a recognized treatment for post-menopausal osteoporosis.

In addition to bone mass, estrogen appears to have an effect on the biosynthesis of cholesterol and cardiovascular health. Statistically, the rate of occurrence of cardiovascular disease is roughly equal in postmenopausal women and men; however, premenopausal women have a much lower incidence of cardiovascular disease than men. Because postmenopausal women are estrogen deficient, it is believed that estrogen plays a beneficial role in preventing cardiovascular disease. The mechanism is not well understood, but evidence indicates that estrogen can upregulate the low density lipid (LDL) cholesterol receptors in the liver to remove excess cholesterol.

Postmenopausal women given estrogen replacement therapy experience a return of lipid levels to concentrations comparable to levels associated with the premenopausal state. Thus, estrogen replacement therapy could be an effective treatment for such disease. However, the side effects associated with long term estrogen use limit the use of this alternative.

Other disease states that affect postmenopausal women include estrogen-dependent breast cancer and uterine cancer. Anti-estrogen compounds, such as tamoxifen, have commonly been used as chemotherapy to treat breast cancer patients. Tamoxifen, a dual antagonist and agonist of estrogen receptors, is beneficial in treating estrogen-dependent breast cancer. However, treatment with tamoxifen is less than ideal because tamoxifen's agonist behavior enhances its unwanted estrogenic side effects. For example, tamoxifen and other compounds that agonize estrogen receptors tend to increase cancer cell production in the uterus. A better therapy for such cancers would be an anti-estrogen compound that has negligible or nonexistent agonist properties.

Although estrogen can be beneficial for treating pathologies such as bone loss, increased lipid levels, and cancer, long-term estrogen therapy has been implicated in a variety of disorders, including an increase in the risk of uterine and endometrial cancers. These and other side effects of estrogen replacement therapy are not acceptable to many women, thus limiting its use.

Alternative regimens, such as a combined progestogen and estrogen dose, have been suggested in an attempt to lessen the risk of cancer. However, such regimens cause the patient to experience withdrawal bleeding, which is unacceptable to many older women. Furthermore, combining estrogen with progestogen reduces the beneficial cholesterol-lowering effect of estrogen therapy. In addition, the long term effects of progestogen treatment are unknown.

In addition to post-menopausal women, men suffering from prostatic cancer can also benefit from anti-estrogen compounds. Prostatic cancer is often endocrine-sensitive; androgen stimulation fosters tumor growth, while androgen suppression retards tumor growth. The administration of estrogen is helpful in the treatment and control of prostatic cancer because estrogen administration lowers the level of gonadotropin and, consequently, androgen levels.

The estrogen receptor has been found to have two forms: ERα and ERβ. Ligands bind differently to these two forms, and each form has a different tissue specificity to binding ligands. Thus, it is possible to have compounds that are selective for ERα or ERβ, and therefore confer a degree of tissue specificity to a particular ligand.

What is needed in the art are compounds that can produce the same positive responses as estrogen replacement therapy without the negative side effects. Also need are estrogen-like compounds that exert selective effects on different tissues of the body.

The compounds of the instant invention are ligands for estrogen receptors and as such may be useful for treatment or prevention of a variety of conditions related to estrogen functioning including: bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restinosis, gynacomastia, vascular smooth muscle cell proliferation, obesity, incontinence, and cancer, in particular of the breast, uterus and prostate.

### SUMMARY OF THE INVENTION

The present invention relates to compounds of the following chemical formula:

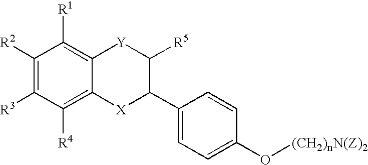

wherein R¹, R², R³, and R⁴ are each independently selected from the group consisting of hydrogen, C₁₋₅ alkyl, C₃₋₈ cycloalkyl, C₂₋₅ alkenyl, C₂₋₅ alkynyl, C₃₋₈ cycloalkenyl, phenyl, heteroaryl, heterocyclical, CF₃, —OR⁶, halogen, C₁₋₅ alkylthio, thiocyanato, cyano, —CO₂H, —COOC₁₋₅ alkyl, —COC₁₋₅ alkyl, —CONZ₂, —SO₂NZ₂, and —SO₂C₁₋₅ alkyl, wherein said alkyl, alkenyl, alkynyl, cycloalkyl, cycloalkenyl, phenyl, heteroaryl, heterocyclical groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, —CO₂H, —COOC₁₋₅ alkyl, —COC₁₋₅ alkyl, —CONZ₂, —SO₂NZ₂, and —SO₂C₁₋₅ alkyl;

R⁵ is selected from the group consisting of C₁₋₅ alkyl, C₃₋₈ cycloalkyl, C₂₋₅ alkenyl, C₂₋₅ alkynyl, C₃₋₈ cycloalkenyl, phenyl, heteroaryl, heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, —CO₂H, —COOC₁₋₅ alkyl, —COC₁₋₅ alkyl, —CONZ₂, —SO₂NZ₂, and —SO₂C₁₋₅ alkyl;

X and Y are each independently selected from the group consisting of oxygen, sulfur, sulfoxide and sulfone;

R⁶ is selected from the group consisting of hydrogen, C₁₋₅ alkyl, benzyl, methoxymethyl, triorganosilyl, C₁₋₅ alkylcarbonyl, alkoxycarbonyl and CONZ₂;

Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl;

Each n is independently an integer from one to five;

and the pharmaceutically acceptable salts thereof.

The present invention also relates to pharmaceutical compositions comprising the compounds of the present invention and a pharmaceutically acceptable carrier.

The present invention also relates to methods for making the pharmaceutical compositions of the present invention.

The present invention also related to processes and intermediates useful for making the compounds and pharmaceutical compositions of the present invention.

The present invention also relates to methods for eliciting an estrogen receptor modulating effect in a mammal in need thereof by administering the compounds and pharmaceutical compositions of the present invention.

The present invention also relates to methods for eliciting an estrogen receptor antagonizing effect in a mammal in need thereof by administering the compounds and pharmaceutical compositions of the present invention. The estrogen receptor antagonizing effect can be either an ERα antagonizing effect, and ERβ antagonizing effect or a mixed ERα and ERβ antagonizing effect.

The present invention also relates to methods for eliciting an estrogen receptor agonizing effect in a mammal in need thereof by administering the compounds and pharmaceutical compositions of the present invention. The estrogen receptor agonizing effect can be either an ERα agonizing effect, and ERβ agonizing effect or a mixed ERα and ERβ agonizing effect.

The present invention also relates to methods for treating or preventing disorders related to estrogen functioning, bone loss, bone fractures, osteoporosis, cartilage degeneration, endometriosis, uterine fibroid disease, cancer of the breast, uterus or prostate, hot flashes, cardiovascular disease, impairment of cognitive function, cerebral degenerative disorders, restenosis, gynacomastia, vascular smooth muscle dell proliferation, obesity and incontinence in a mammal in need thereof by administering the compounds and pharmaceutical compositions of the present invention.

The present invention also relates to methods for reducing bone loss, lowering LDL cholesterol levels and eliciting a vasodilatory effect, in a mammal in need thereof by administering the compounds and pharmaceutical compositions of the present invention.

### DETAILED DESCRIPTION OF THE INVENTION

The present invention relates to compounds useful as estrogen receptor modulators. Compounds of the present invention are described by the following chemical formula:

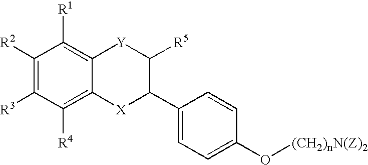

wherein R¹, R², R³, and R⁴ are each independently selected from the group consisting of hydrogen, C₁₋₅ alkyl, C₃₋₈ cycloalkyl, C₂₋₅ alkenyl, C₂₋₅ alkynyl, C₃₋₈ cycloalkenyl, phenyl, heteroaryl, heterocyclical, CF₃, —OR⁶, halogen, C₁₋₅ alkylthio, thiocyanato, cyano, —CO₂H, —COOC₁₋₅ alkyl, —COC₁₋₅ alkyl, —CONZ₂, —SO₂NZ₂, and —SO₂C₁₋₅ alkyl, wherein said alkyl, alkenyl, alkynyl, cycloalkyl, cycloalkenyl, phenyl, heteroaryl, heterocyclical groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, —CO₂H, —COOC₁₋₅ alkyl, —COC₁₋₅ alkyl, —CONZ₂, —SO₂NZ₂, and —SO₂C₁₋₅ alkyl;

R⁵ is selected from the group consisting of C₁₋₅ alkyl, C₃₋₈ cycloalkyl, C₂₋₅ alkenyl, C₂₋₅ alkynyl, C₃₋₈ cycloalkenyl, phenyl, heteroaryl, heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, —CO₂H, —COOC₁₋₅ alkyl, —COC₁₋₅ alkyl, —CONZ₂, —SO₂NZ₂, and —SO₂C₁₋₅ alkyl;

X and Y are each independently selected from the group consisting of oxygen, sulfur, sulfoxide and sulfone;

R⁶ is selected from the group consisting of hydrogen, C₁₋₅ alkyl, benzyl, methoxymethyl, triorganosilyl, C₁₋₅ alkylcarbonyl, alkoxycarbonyl and CONZ₂;

Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl;

Each n is independently an integer from one to five;

and the pharmaceutically acceptable salts thereof.

In one class of compounds of the present invention, X is O, and Y is S. In another class of compounds of the present invention, X is S and Y is S.

In one class of compounds of the present invention, R¹, R² , R³ and R⁴ are selected from the group consisting of hydrogen, C₁₋₅ alkyl, C₃₋₈ cycloalkyl, C₁₋₅ alkenyl, C₁₋₅ alkynyl, —OR⁶ and halogen.

In one class of compounds of the present invention R⁵ is selected from the group consisting of C₃₋₈ cycloalkyl, phenyl, heteroaryl and heterocyclical groups wherein said groups can be optionally substituted with —OR⁶ and halogen.

In one class of compounds of the present invention, R⁶ is preferably selected from the group consisting of hydrogen, C₁₋₅ alkyl, benzyl, methoxymethyl and triisopropylsilyl.

The present invention also relates to a process for preparing a compound of formula I

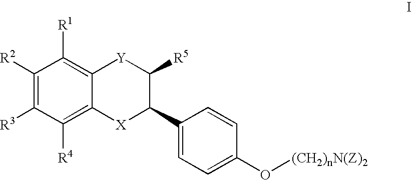

wherein R¹ is H, F, or Cl;

R² is H or OR⁶;

R³ is H or OR⁶;

R⁴ is H or CH₃;

R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SO₂NZ₂), and sulfonyl (—SO₂C₁₋₅ alkyl);

R⁶ is H, benzyl, methyl, methoxymethyl, or triisopropylsilyl, with the proviso that when OR⁶ exists elsewhere, it is chemically differentiable;

X and Y are each independently selected from the group consisting of oxygen, sulfur, sulfoxide and sulfone;

Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl;

n is an integer from one to five;

and the stereoisomer is cis;

or a pharmaceutically acceptable salt thereof,

comprising the steps of

a) reacting a compound of formula II with a compound of formula III under basic conditions

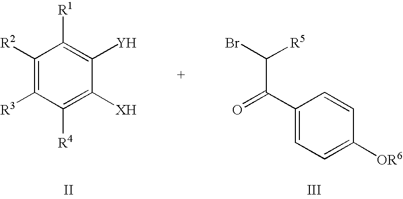

to form a compound of formula IV

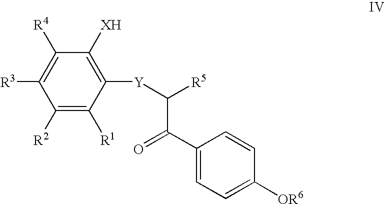

b) cyclizing IV, of step a, under acidic conditions in the presence of a reducing agent, to provide the cis compound of formula V

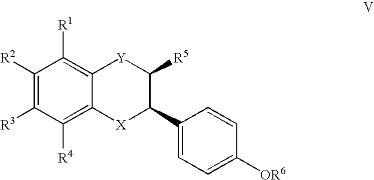

c) removing the protecting group R⁶ to yield the substituted phenol of formula VI

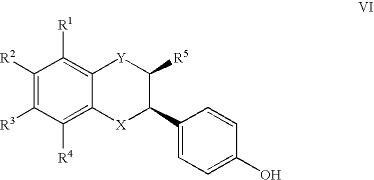

d) alkylating the substituted phenol of formula VI, from step c, with a reagent, HO(CH₂)ₙN(Z)₂, to give a compound of formula I

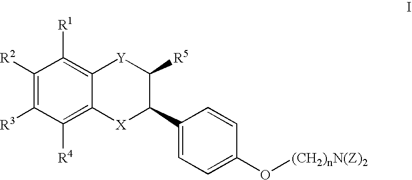

e) removing either protecting group from I, from step d, to afford either a compound of formula VIII or a compound of formula IX

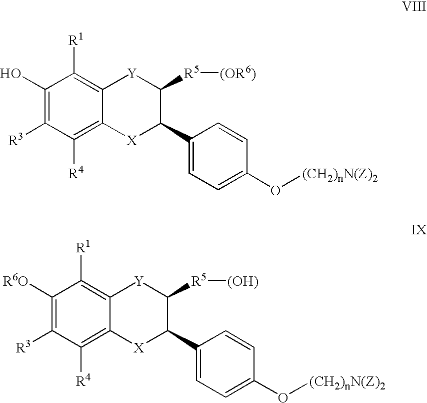

f) removing the remaining protecting group from either VIII or IX, from step e, to give a compound of formula I.

The present invention also relates to a process for preparing a compound of formula ID

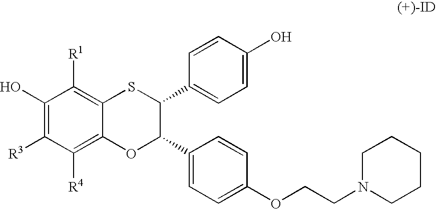

wherein R¹ is H, F, or Cl;

R³ is H;

R⁴ is H or CH₃;

and the stereoisomer is cis;

and the optical isomer is dextrorotatory (+), having the absolute configuration: (2S, 3R);

or a pharmaceutically acceptable salt thereof,

comprising the steps of

a) reacting a compound of formula IID with a compound of formula IIID under basic conditions

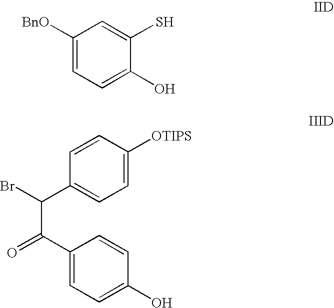

to form a compound of formula IVD

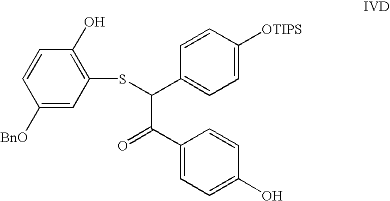

b) cyclizing IVD, of step a, under acidic conditions in the presence of a reducing agent to provide the racemic, cis compound of formula VD

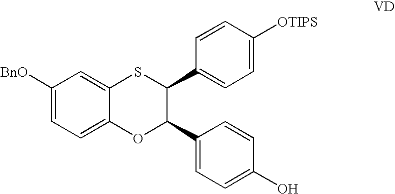

c) performing a chiral chromatography with VD, from step b, to resolve the enantiomeric forms to provide the dextrorotatory (+) isomer VID;

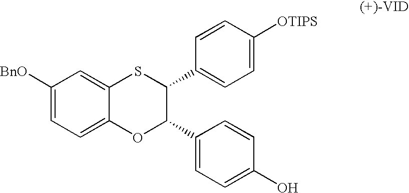

d) alkylating the dextrorotatory (+) isomer VID, from step c, with 1-piperidineethanol to give a compound of formula VIID

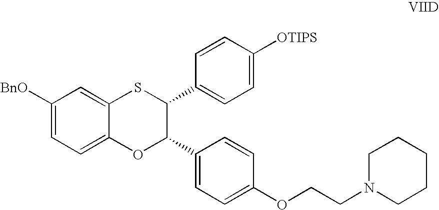

e) removing either protecting group from VIID, from step d, to afford either a compound of formula VIIID or a compound of formula IXD

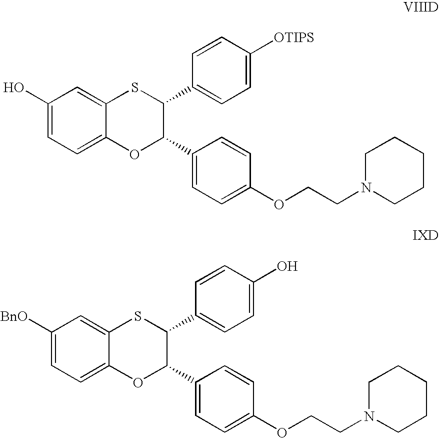

f) removing the remaining protecting group from either VIIID or IXD, from step e, to give a compound of formula I.

The present invention also comprises a process according for preparing a compound of formula IE

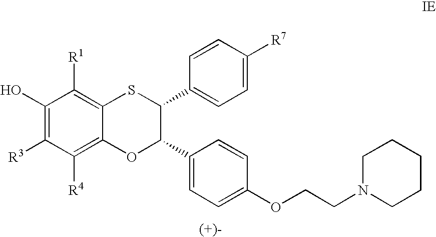

wherein

R¹ is selected from the group consisting of H, F, or Cl;

R³ and R⁴ are each H;

R⁷ is selected from the group consisting of H or OH;

the stereoisomer is cis, and the optical isomer is dextrorotatory (+), having the absolute configuration (2S, 3R);

or a pharmaceutically acceptable salt thereof

comprising the steps of

a) reacting a compound of formula IIE with a compound of formula IIIE under basic conditions

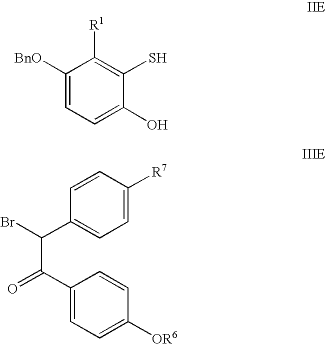

to form a compound of formula IVE

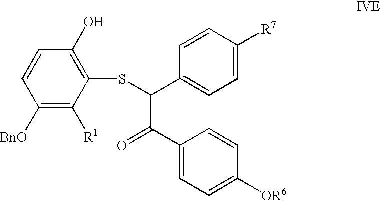

b) cyclizing IVE, of step a, under acidic conditions in the presence of a reducing agent to provide the racemic, cis compound of formula VE

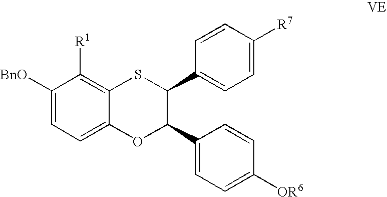

c) selectively removing the protecting group of VE, from step b, to yield the substituted phenol of formula VIE

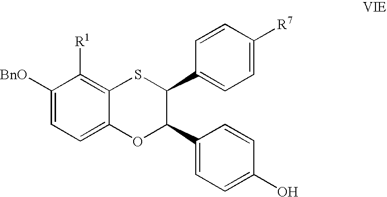

d) alkylating the substituted phenol of formula VIE, from step c, with 1-piperidineethanol to give a compound of formula VIIE

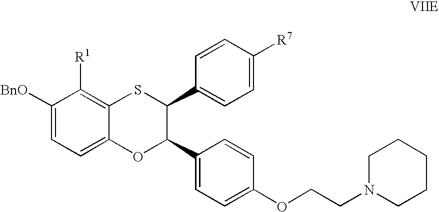

e) removing either protecting group from VIIE to afford either a compound of formula VIIIE or a compound of formula IXE

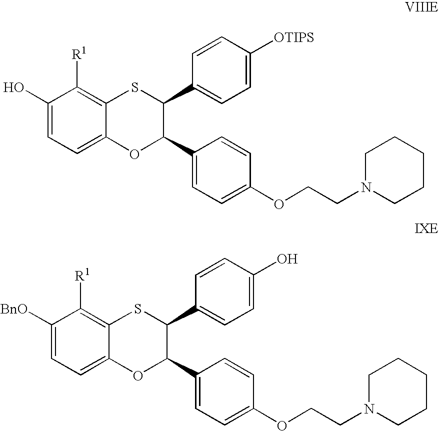

f) removing the remaining protecting group from either VIII or IX, from step e, to provide racemic I.

g) performing a resolution of the enantiomeric forms of I to provide the dextrorotatory (+) isomer I, having the (2S, 3R) absolute configuration.

The present invention also relates to novel intermediates useful for preparing compounds and compositions described herein, i.e. compounds of formula I, IA, IB, IC, ID and IE.

An embodiment of the invention is an intermediate of the formula:

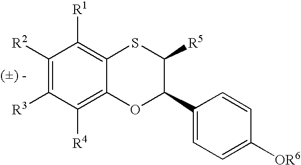

wherein R¹ is H, F, or Cl;

R² is H or OR⁶;

R³ is H or OR⁶;

R⁴ is H or CH₃;

R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SO₂NZ₂), and sulfonyl (—SO₂C₁₋₅ alkyl);

R⁶ is H, benzyl, methyl, methoxymethyl, or trisopropylsilyl, with the proviso that when OR⁶ exists elsewhere, it is chemically differentiable;

Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C_{1 5} alkyl;

Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl.

Another embodiment of the invention is an intermediate of the formula:

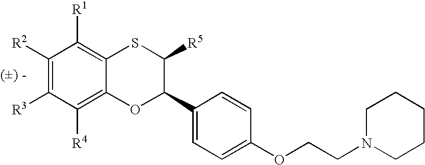

wherein R¹ is H, F, or Cl;

R² is H or OR⁶;

R³ is H or OR⁶;

R⁴ is H or CH₃;

R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SO₂NZ₂), and sulfonyl (—SO₂C₁₋₅ alkyl);

R⁶ is H, benzyl, methyl, methoxymethyl, or triisopropylsilyl, with the proviso that when OR⁶ exists elsewhere, it is chemically differentiable;

Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl.

Another embodiment of the invention is an intermediate of the formula:

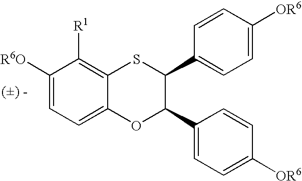

wherein R¹ is H, F, or Cl;

R⁶ is H, benzyl, methyl, methoxymethyl, or triisopropylsilyl, with the proviso that all existing R⁶ groups are chemically differentiable.

Another embodiment of the invention is an intermediate of the formula:

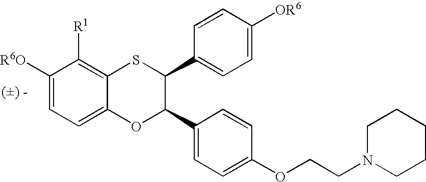

wherein R¹ is H, F, or Cl;

R⁶ is H, benzyl, methyl, methoxymethyl, or trisopropylsilyl, with the proviso that all existing R⁶ groups are chemically differentiable.

Another embodiment of the invention is an intermediate of the formula:

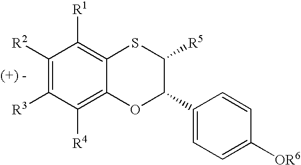

wherein R¹ is H, F, or Cl;

R² is H or OR⁶;

R³ is H or OR⁶;

R⁴ is H or CH₃;

R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SO₂NZ₂), and sulfonyl (—SO₂C₁₋₅ alkyl);

R⁶ is H, benzyl, methyl, methoxymethyl, or triisopropylsilyl, with the proviso that when OR⁶ exists elsewhere, it is chemically differentiable;

Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;

Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl.

Another embodiment of the invention is an intermediate of the formula:

wherein R¹ is H, F, or Cl;

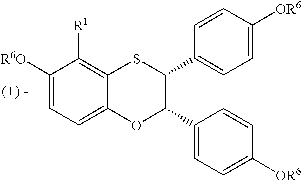

R⁶ is H, benzyl, methyl, methoxymethyl, or triisopropylsilyl, with the proviso that all existing R⁶ groups are chemically differentiable.

Another embodiment of the present invention is an intermediate of the formula:

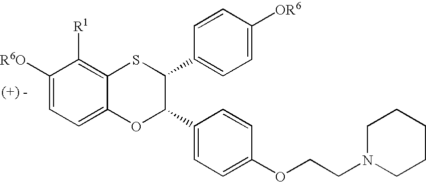

wherein R¹ is H, F, or Cl;

R⁶ is H, benzyl, methyl, methoxymethyl, or triisopropylsilyl, with the proviso that all existing R⁶ groups are chemically differentiable.

An embodiment of the invention is a method of eliciting an estrogen receptor modulating effect in a mammal in need thereof, comprising administering to the mammal a therapeutically effective amount of any of the compounds or any of the above pharmaceutical compositions described above.

A class of the embodiment is the method wherein the estrogen receptor modulating effect is an antagonizing effect.

A subclass of the embodiment is the method wherein the estrogen receptor is an ERα receptor.

A second subclass of the embodiment is the method wherein the estrogen receptor is an ERβ receptor.

A third subclass of the embodiment is the method wherein the estrogen receptor modulating effect is a mixed ERα and ERβ receptor antagonizing effect.

A second class of the embodiment is the method wherein the estrogen receptor modulating effect is an agonizing effect.

A subclass of the embodiment is the method wherein the estrogen receptor is an ERα receptor.

A second subclass of the embodiment is the method wherein the estrogen receptor is an ERβ receptor.

A third subclass of the embodiment is the method wherein the estrogen receptor modulating effect is a mixed ERα and ERβ receptor agonizing effect.

Another embodiment of the invention is a method of treating or preventing post-menopausal osteoporosis in a mammal in need thereof by administering to the mammal a therapeutically effective amount of any of the compounds or pharmaceutical compositions described above.

Another embodiment of the invention is a method of treating or preventing uterine fibroids in a mammal in need thereof by administering to the mammal a therapeutically effective amount of any of the compounds or pharmaceutical compositions described above.

Another embodiment of the invention is a method of treating or preventing restenosis in a mammal in need thereof by administering to the mammal a therapeutically effective amount of any of the compounds or pharmaceutical compositions described above.

Another embodiment of the invention is a method of treating or preventing endometriosis in a mammal in need thereof by administering to the mammal a therapeutically effective amount of any of the compounds or pharmaceutical compositions described above.

Another embodiment of the invention is a method of treating or preventing hyperlipidemia in a mammal in need thereof by administering to the mammal a therapeutically effective amount of any of the compounds or pharmaceutical compositions described above.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

Further exemplifying the invention is the use of any of the compounds described above in the preparation of a medicament for the treatment and/or prevention of osteoporosis in a mammal in need thereof. Still further exemplifying the invention is the use of any of the compounds described above in the preparation of a medicament for the treatment and/or prevention of: bone loss, bone resorption, bone fractures, cartilage degeneration, endometriosis, uterine fibroid disease, breast cancer, uterine cancer, prostate cancer, hot flashes, cardiovascular disease, impairment of cognitive functioning, cerebral degenerative disorder, restenosis, vascular smooth muscle cell proliferation, incontinence, and/or disorders related to estrogen functioning.

The present invention is also directed to combinations of any of the compounds or any of the pharmaceutical compositions described above with one or more agents useful in the prevention or treatment of osteoporosis. For example, the compounds of the instant invention may be effectively administered in combination with effective amounts of other agents such as an organic bisphosphonate or a cathepsin K inhibitor. Nonlimiting examples of said organic bisphosphonates include alendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof. Preferred organic bisphosphonates include alendronate and pharmaceutically acceptable salts and mixtures thereof. Most preferred is alendronate monosodium trihydrate.

The precise dosage of the bisphosphonate will vary with the dosing schedule, the oral potency of the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis, i.e. on the basis of the corresponding acid.

For use in medicine, the salts of the compounds of this invention refer to non-toxic “pharmaceutically acceptable salts.” Other salts may, however, be useful in the preparation of the compounds according to the invention or of their pharmaceutically acceptable salts. When the compounds of the present invention contain a basic group, salts encompassed within the term “pharmaceutically acceptable salts” refer to non-toxic salts which are generally prepared by reacting the free base with a suitable organic or inorganic acid. Representative salts include but are not limited to the following: acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, palmoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds of the present invention can have chiral centers and occur as racemates, racemic mixtures, diastereomeric mixtures, and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

The term “therapeutically effective amount” shall mean that amount of a drug or pharmaceutical agent that will elicit the biological or medical response of a tissue, system, animal or human that is being sought by a researcher or clinician.

The term “bone resorption,” as used herein, refers to the process by which osteoclasts degrade bone.

The term “basic conditions,” as used herein, refers to the incorporation or use of a base in the reaction medium. According to the Lowry-Bronsted definition, a base is a substance that accepts a proton; or according to the Lewis definition, a base is a substance that can furnish an electron pair to form a covalent bond. Examples of bases used herein, but are not limited to, are tertiary amine bases such as triethylamine, diisopropylethylamine, or the like.

The term “acidic conditions,” as used herein, refers to the incorporation or use of an acid in the reaction medium. According to the Lowry-Bronsted definition, an acid is a substance that gives up a proton; or according to the Lewis definition, an acid is a substance that can take up an electron pair to form a covalent bond. Examples of acids used herein, but are not limited to, are strong carboxylic acids such as trifluoroacetic acid, or the like, strong sulfonic acids, such as trifluoromethane sulfonic acid, or the like, and Lewis acids, such as boron trifluoride etherate, or stannous chloride, or the like.

The term “reducing agent,” as used herein, refers to a reagent capable of performing a reduction. A reduction is the conversion of a functional group or an intermediate from one category to a lower one. Examples of reducing agents used herein, but are not limited to, are triorganosilanes or stannanes, such as triethylsilane, triphenylsilane, and tri-n-butyl tin hydride, or the like.

The term “chemically differentiable” refers to two or more non-identical R⁶ substituents whose unique structures are such that one of ordinary skill in the art could choose reaction conditions which would convert one of the non-identical R⁶ substituents to H, without affecting the other R⁶ substituent.

The term “alkyl” shall mean a substituting univalent group derived by conceptual removal of one hydrogen atom from a straight or branched-chain acyclic saturated hydrocarbon (i.e., —CH₃, —CH₂CH₃, —CH₂CH₂CH₃, —CH(CH₃)₂, —CH₂CH₂CH₂CH₃, —CH₂CH(CH₃)₂, —C(CH₃)₃, etc.).

The term “alkenyl” shall mean a substituting univalent group derived by conceptual removal of one hydrogen atom from a straight or branched-chain acyclic unsaturated hydrocarbon containing at least one double bond (i.e., —CH═CH₂, —CH₂CH═CH₂, —CH═CHCH₃, —CH₂CH═C(CH₃)₂, etc.).

The term “alkynyl” shall mean a substituting univalent group derived by conceptual removal of one hydrogen atom from a straight or branched-chain acyclic unsaturated hydrocarbon containing at least one triple bond (i.e., —CH═CH, —CH₂C═CH, —C═CCH₃, —CH₂CH₂C═CCH₃, etc.).

The term “cycloalkyl” shall mean a substituting univalent group derived by conceptual removal of one hydrogen atom from a saturated monocyclic hydrocarbon (i.e., cyclopropyl, cyclobutyl, cyclopentyl, cyclohexyl, or cycloheptyl).

The term “cycloalkenyl” shall mean a substituting univalent group derived by conceptual removal of one hydrogen atom from an unsaturated monocyclic hydrocarbon containing a double bond (i.e., cyclopentenyl or cyclohexenyl).

The term “heterocyclical” shall mean a substituting univalent group derived by conceptual removal of one hydrogen atom from a heterocycloalkane wherein said heterocycloalkane is derived from the corresponding saturated monocyclic hydrocarbon by replacing one or two carbon atoms with atoms selected from N, O or S. Examples of heterocyclical groups include, but are not limited to, oxiranyl, azetidinyl, pyrrolidinyl, piperidinyl, piperazinyl, and morpholinyl. Heterocyclical substituents can be attached at a carbon atom. If the substituent is a nitrogen containing heterocyclical substituent, it can be attached at the nitrogen atom.

The term “heteroaryl” as used herein refers to a substituting univalent group derived by the conceptual removal of one hydrogen atom from a monocyclic or bicyclic aromatic ring system containing 1, 2, 3, or 4 heteroatoms selected from N, O, or S. Examples of heteroaryl groups include, but are not limited to, pyrrolyl, furyl, thienyl, imidazolyl, pyrazolyl, oxazolyl, isoxazolyl, thiazolyl, pyridyl, pyrimidinyl, pyrazinyl, benzimidazolyl, indolyl, and purinyl. Heteraryl substituents can be attached at a carbon atom or through the heteroatom.

The term “triorganosilyl” means those silyl groups trisubstituted by lower alkyl groups or aryl groups or combinations thereof and wherein one substituent may be a lower alkoxy group. Examples of triorganosilyl groups include trimethylsilyl, triethylsilyl, t-butyldimethylsilyl, triisopropylsilyl, triphenylsilyl, dimethylphenylsilyl, t-butyldiphenylsilyl, phenyl-t-butylmethoxysilyl and the like.

In the compounds of the present invention, alkyl, alkenyl, alkynyl, cycloalkyl, cycloalkenyl, heterocyclical and heteroaryl groups can be further substituted by replacing one or more hydrogen atoms be alternative non-hydrogen groups. These include, but are not limited to, halo, hydroxy, mercapto, amino, carboxy, cyano and carbamoyl.

Whenever the term “alkyl” or “aryl” or either of their prefix roots appear in a name of a substituent (e.g., aryl C₀₋₈ alkyl) it shall be interpreted as including those limitations given above for “alkyl” and “aryl.” Designated numbers of carbon atoms (e.g., C₁₋₁₀) shall refer independently to the number of carbon atoms in an alkyl or cyclic alkyl moiety or to the alkyl portion of a larger substituent in which alkyl appears as its prefix root.

The terms “arylalkyl” and “alkylaryl” include an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, chlorophenylethyl, thienylmethyl, thienylethyl, and thienylpropyl. Examples of alkylaryl include, but are not limited to, toluyl, ethylphenyl, and propylphenyl.

The term “heteroarylalkyl,” as used herein, shall refer to a system that includes a heteroaryl portion, where heteroaryl is as defined above, and contains an alkyl portion. Examples of heteroarylalkyl include, but are limited to, pyridylmethyl, pyridylethyl and imidazoylmethyl.

The term “halo” shall include iodo, bromo, chloro and fluoro.

The term “oxy” means an oxygen (O) atom. The term “thio” means a sulfur (S) atom. The term “oxo” means ═O. The term “oximino” means the ═N—O group.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to

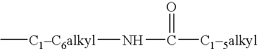

In choosing compounds of the present invention, one of ordinary skill in the art will recognize that the various substituents, i.e. R¹, R², R³, R⁴, R⁵, R⁶, R⁷, Q, X, Y, Z, n and m are to be chosen in conformity with well-known principles of chemical structure connectivity.

Representative compounds of the present invention typically display submicromolar affinity for alpha and/or beta estrogen receptors. Compounds of this invention are therefore useful in treating mammals suffering from disorders related to estrogen functioning. Pharmacologically effective amounts of the compound, including the pharmaceutically effective salts thereof, are administered to the mammal, to treat disorders related to estrogen functioning, such as bone loss, hot flashes and cardiovascular disease.

The compounds of the present invention are available in racemic form or as individual enantiomers. For convenience, some structures are graphically represented as a single enantiomer but, unless otherwise indicated, is meant to include both racemic and enantiomeric forms. Where cis and trans sterochemistry is indicated for a compound of the present invention, it should be noted that the stereochemistry can be construed as relative, unless indicated otherwise.

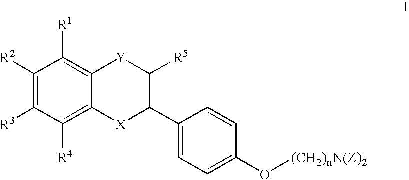

It is generally preferable to administer compounds of structure (I) as enantiomerically pure formulations since most or all of the desired bioactivity resides with a single enantiomer. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

The compounds of the present invention can be used in combination with other agents useful for treating estrogen-mediated conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating estrogen-mediated conditions includes in principle any combination with any pharmaceutical composition useful for treating disorders related to estrogen functioning.

As used herein, the term “composition” is intended to encompass a product comprising the specified ingredients in the specified amounts, as well as any product which results, directly or indirectly, from combination of the specified ingredients in the specified amounts.

The compounds of the present invention can be administered in such oral dosage forms as tablets, capsules (each of which includes sustained release or timed release formulations), pills, powders, granules, elixers, tinctures, suspensions, syrups and emulsions. Likewise, they may also be administered in intravenous (bolus or infusion), intraperitoneal, topical (e.g., ocular eyedrop), subcutaneous, intramuscular or transdermal (e.g., patch) form, all using forms well known to those of ordinary skill in the pharmaceutical arts.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The novel compounds of the present invention can be prepared according to the procedure of the following schemes and examples, using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

The compounds of the present invention are prepared according to the following generic Scheme I:

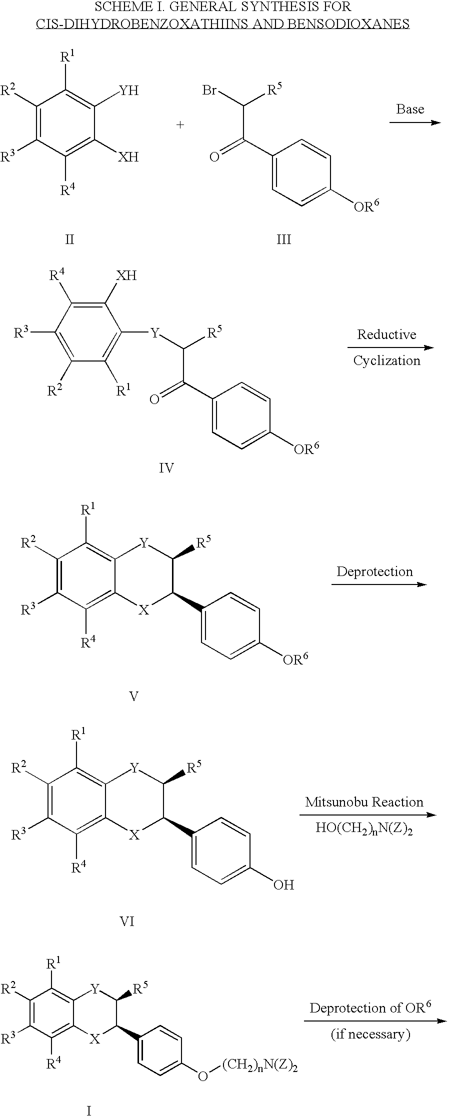

In words relative to the scheme, an appropriately functionalized bis-phenol II (X=O, Y=O), which are readily available, or a mercapto-phenol II (X=O, Y=S), which are prepared according to literature procedures, was reacted with a bromo-ketone derivative III, which was readily prepared from the corresponding ketone by bromination with phenyltrimethylammonium tribromide (PTAB), in the presence of a tertiary amine base, such as triethylamine, diisopropylethylamine, or the like, in a solvent such as dimethylformamide (DMF), formamide, acetonitrile, dimethylsulfoxide (DMSO), tetrahydrofuran (THF), dichloromethane, or the like, at a temperature of from −20° C. to 80° C. for as long as it takes for the reaction to complete to provide the displacement product IV. When X=Y=O, only R³ maybe —OR⁶. Alternatively, when X=Y=O and R² is —OR⁶, the requisite cyclization intermediate is obtained by interchangement of the ketone and bromide functionalities. These stipulations are required to allow for the preparation of these compounds of the invention where the presence of certain substituents will alter the reactivity of the phenolic oxygen atoms.

Intermediate IV was reductively cyclized in the presence of an organic acid such as trifluoroacetic acid, triflic acid, or the like, or a Lewis acid such as boron trifluoride etherate, stannous chloride, or the like, and a reducing agent such as a trisubstituted silane, such triethylsilane, or the like, in a solvent such as dichloromethane, chloroform, THF, toluene, or the like at a temperature of from −40° C. to 100° C. for as long as it takes for the reaction to complete to provide the cyclized product V, in which the stereochemistry of the aryl substituent and R⁵ in the newly created ring is exclusively cis. The formation of the intermediates with analogous trans stereochemistry is depicted in the next general Scheme II.

In product V, when R⁶ is a protecting group it is then removed in a manner consistent with its nature. Such methods are well documented in the literature which are incorporated in standard textbooks, such as Greene, T. W. and Wuts, P. G. M., Protective Groups in Organic Synthesis, Third Ed., Wiley, New York (1999). Further, it is understood that it is possible to have any number of the substituents R¹-R⁴ be or contain —OR⁶, or R⁵ may contain —OR⁶, where R⁶ is a protecting group, and it is further understood that in these instances the protecting groups are chemically differentiable, i.e., they maybe selectively removed when necessary. For example in product V, R⁶ is a methoxymethyl (OM) group, R² is —OR⁶, wherein R⁶ is a benzyl (Bn) group, R⁵ is a phenyl ring substituted by R⁷ where R⁷ is OR⁶, wherein R⁶ is a triisopropylsilyl (TIPS) group, and all unspecified substitutents are hydrogen. As indicated, as part of the synthetic sequence it is necessary to selectively remove the MOM group in preference to either the TIPS or Bz groups. Utilizing methods found in Green and Wuts, it is possible to generate the preferred intermediate V, wherein R⁶ is H, R² is —OBn, R⁵ is para-OTIPS-phenyl, and all unspecified substitutents are hydrogen. It is also noted that in product V, that when either R² or R³ is OR⁶, R⁶ must be a protecting group, and that prior to its removal, the existing —OR⁶ group must be covered by a differentiable protecting group.

The alcohol intermediate VI was then reacted with a reagent HO(CH₂)ₙNZ₂ in a Mitsunobu reaction protocol, in which they are combined with a trisubstituted phosphine, such as triphenylphosphine and a diazodicarboxylate, such as diisopropylazodicarboxylate, in a suitable solvent such as THF at from 0° C. to 80° C. for as long as it takes for the reaction to complete to provide the coupled product I. The variables for the Mitsunobu reaction have been well documented and are incorporated herein by reference: Mitsunobu, O. Synthesis, 1981, 1; Castro, B. R. Org. React. 1983, 29, 1; Hughes, D. L. Org. React. 1992, 42, 335.

Finally, after the Mitsunobu reaction, it is understood that in I if any R group is or contains —OR⁶, wherein R⁶ is a protecting group, it was removed utilizing the appropriate method found in Green and Wuts to give the final product where R⁶ is H.

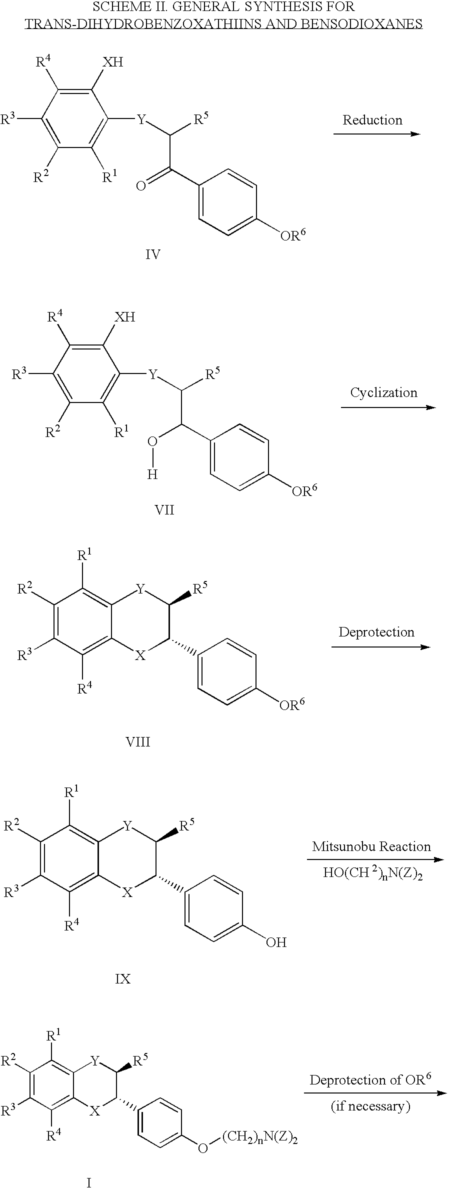

In words relative to the above scheme for the general preparation of the trans isomers of I, the ketone intermediate IV from Scheme I was reduced with sodium borohydride, super hydride, or the like, in a mixture of methanol and dichloromethane, or THF or the like at from 0° C. to ambient temperature for from a few minutes to a few hours to provide the analogous hydroxyl intermediate VII.

Cyclization of intermediate VII was accomplished in the presence of an acid catalyst such as amberlyst 15, or triflic acid or the like, in a solvent such toluene, or dichloromethane or the like, at a temperature of from ambient to reflux to afford the trans compound VIII as the major isomer.

The remainder of the synthetic sequence to produce trans I is identical to that outlined in Scheme I and detailed above.

The compounds of the invention where X=O and Y=SO or SO₂ are prepared as outlined in the specific schemes that follow.

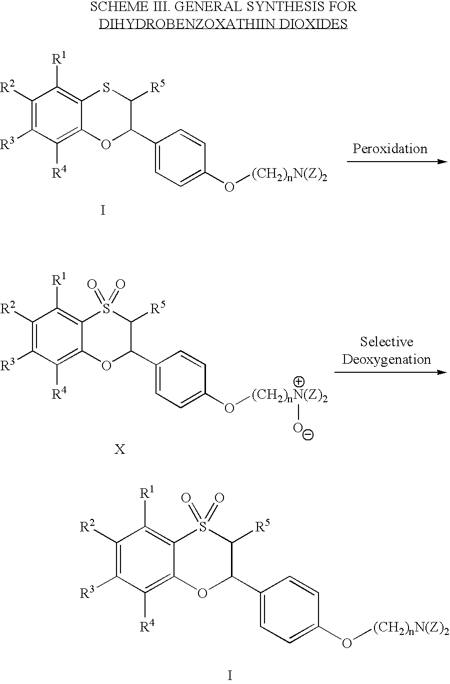

In words relative to Scheme III, the compounds I of the invention are peroxidized with an oxidant such as m-chloroperbenzoic acid, or per-trifluoroacetic acid, or the like, in a solvent such dichloromethane or the like, at a temperature of from 0° C. to reflux to produce the trioxide intermediate X. In turn X was selectively deoxygenated at the nitrogen atom by treatment with a reducing agent such as sodium bisulfite or the like in a biphasic medium such as ethyl acetate and water, or the like, to provide I.

In the compounds of the present invention, X is preferably O, and Y is preferably S.

In the compounds of the present invention, R¹, R², R³ and R⁴ are preferably selected from the group consisting of hydrogen, C₁₋₅ alkyl, C₃₋₈ cycloalkyl, C₁₋₅ alkenyl, C₁₋₅ alkynyl, —OR⁶ and halogen.

In the compounds of the present invention, R⁵ is preferably selected from the group consisting of C₃₋₈ cycloalkyl, phenyl, and substituted phenyl.

In the compounds of the present invention, R⁶ is preferably selected from the group consisting of hydrogen, C₁₋₅ alkyl, benzyl, methoxymethyl and trisopropylsilyl.

In the compounds of the present invention, a preferred subset is found where R¹ and R⁴ are hydrogen, R² and R³ are independently —OH, and R⁵ is independently selected from the group consisting of phenyl and substituted phenyl.

In the compounds of the present invention, another preferred subset is found where R¹ is independently selected fluorine and chlorine, R⁴ is hydrogen, R² and R³ are independently —OH, and R⁵ is independently selected from the group consisting of phenyl and substituted phenyl.

In the compounds of the present invention, the most preferred subset is found where R¹ and R⁴ are hydrogen and, R² is —OH, and R⁵ is independently selected from the group consisting of phenyl and para-hydroxy-phenyl.

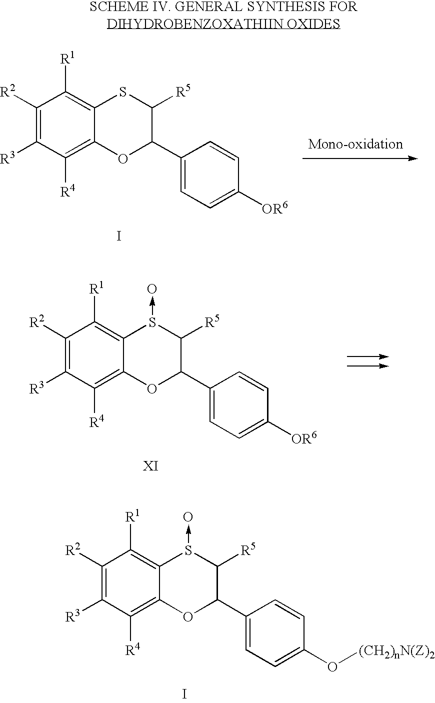

In words relevant to Scheme IV, the intermediate V of Scheme I was mono-oxidized by careful treatment with one equivalent or a slight excess of an oxidant such as m-chloroperbenzoic acid, or dimethyldioxirane, or the like, in a solvent such as dichloromethane, ether, acetone, or the like, at a temperature of from −78° C. to ambient temperature for from a few minutes to a few hours to give the corresponding sulfoxide intermediate XI. The remainder of the synthetic sequence to produce I is identical to that outlined in Scheme I and detailed above.

In the compounds of the present invention, X is preferably O, and Y is preferably S.

In the compounds of the present invention, R¹, R², R³and R⁴ are preferably selected from the group consisting of hydrogen, C₁₋₅ alkyl, C₃₋₈ cycloalkyl, C₁₋₅ alkenyl, C₁₋₅ alkynyl, —OR⁶ and halogen.

In the compounds of the present invention, R⁵ is preferably selected from the group consisting of C₃₋₈ cycloalkyl, phenyl, and substituted phenyl.

In the compounds of the present invention, R⁶ is preferably selected from the group consisting of hydrogen, C₁₋₅ alkyl, benzyl, methoxymethyl and trisopropylsilyl.

In the compounds of the present invention, a preferred subset is found where R¹ and R⁴ are hydrogen, R² and R³ are independently —OH, and R⁵ is independently selected from the group consisting of phenyl and substituted phenyl.

In the compounds of the present invention, another preferred subset is found where R¹ is independently selected fluorine and chlorine, R⁴ is hydrogen, R² and R³ are independently —OH, and R⁵ is independently selected from the group consisting of phenyl and substituted phenyl.

In the compounds of the present invention, the most preferred subset is found where R¹ and R⁴ are hydrogen and, R² is —OH, and R⁵ is independently selected from the group consisting of phenyl, meta-hydroxy-phenyl, and para-hydroxy-phenyl.

### EXAMPLE 1

### General Preparation of Thiophenols

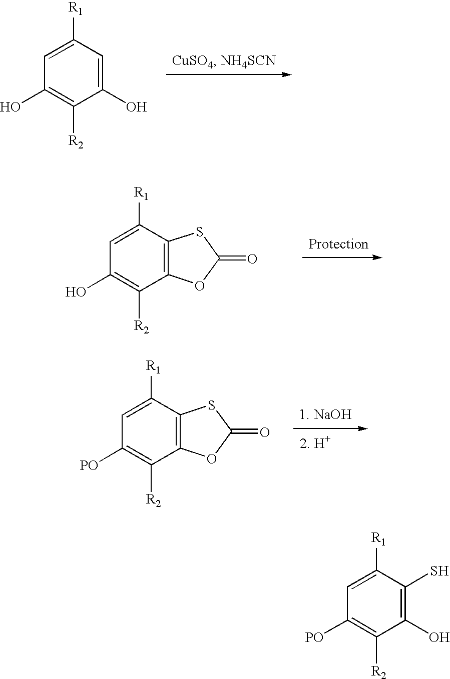

The functionalized thiophenols were prepared by the known procedure, with minor modification, which is depicted in above scheme: Wermer, G.; Biebrich, W. U.S. Pat. Nos. 2,276,553 and 2,332,418.

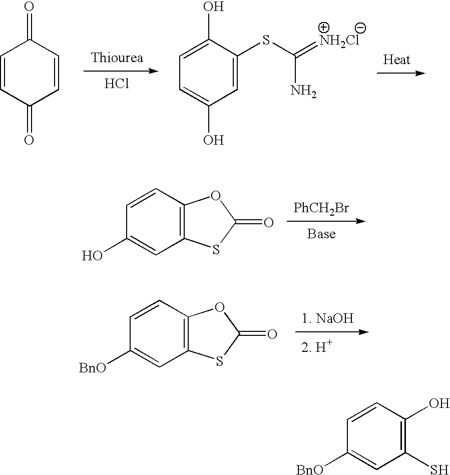

The thiophenol depicted above was prepared according to the following references: Maxwell, S. J. Am. Chem. Soc. 1947, 69, 712; Hanzlik, R. P. et. al. J. Org. Chem. 1990, 55, 2736.

### EXAMPLE 2

### Preparation of 2-thiophene-4-methoxy-benzophenone

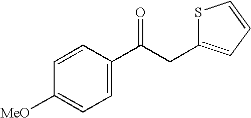

To a stirred solution of anisole (1.49 g, 13.8 mmol) in anhydrous dichloromethane (5 mL) was added AlCl₃ (1.2320 g, 9.2 mmol) followed by dropwise addition of 2-thiophene acetyl chloride (0.57 mL, 4.6 mmol) at 0° C. under N₂. The reaction was stirred for 1.5 h, then poured into a separatory funnel containing ice/brine/EtOAc. The organic layer was washed further with brine, dried over Na₂SO₄, and concentrated in vacuo. The resulting residue was purified by silica gel chromatography with 30% EtOAc/hexane as the eluant to afford the desired product as a yellow oil. ¹H 500 MHz NMR(CDCl₃) ppm(δ): 3.89 (s, 3H), 4.46 (s, 2H), 6.98 (m, 4H), 7.24 (d, 1H), and 8.05 (d, 2H).

### EXAMPLE 3

### Preparation of 2-thiophene-4-hydroxy-benzophenone

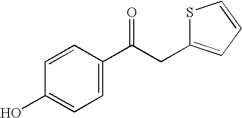

A mixture of the 2-thiophene-4-methoxy-benzophenone (0.8294 g, 3.5 mmol), generated in Example 2, and pyridine-HCl (4.0627 g, 35.2 mmol) was heated to 190° C. under N₂ for 6 h. The reaction was monitored by examining worked-up aliquots of the reaction by TLC (30% EtOAc/hexane). The reaction was cooled in an ice bath and ice/H₂O was added. The resulting mixture was extracted with EtOAc. The organic extract was washed with 2 N HCl and brine, dried over Na₂SO₄, and concentrated in vacuo. The resulting brown residue was purified by silica gel chromatography with 30% EtOAc/hexane as the eluant to afford the desired product as a yellow/orange solid. ¹H 500 MHz NMR(CDCl₃) ppm(δ): 4.43 (s, 2H), 5.60 (bs, 1H), 6.90 (d, 2H), 6.92 (m, 1 H), 6.97 (m, 1H), 7.22 (d, 1 H) and 8.00 (d, 2H).

### EXAMPLE 4

### General Preparation of Cycloalkyl-4-hydroxy-benzophenones

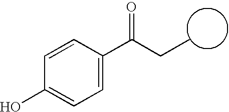

To a stirred solution of the 2-cycloalkyl-1-(4-methoxy-phenyl)-ethanone [prepared according to the method of Barrio, etal, J. Med. Chem., 1971, 14, 898] in dry methylene chloride at 0° C. was added 3.6 equivalents of aluminum chloride and 3.0 equivalents of isopropyl mercaptan. The ice-water bath was removed and the reaction mixture was stirred further overnight under an inert atmosphere of nitrogen. The reaction mixture was poured onto a mixture of 2N HCl/ice and extracted with ethyl acetate. The ethyl acetate extract was washed with brine, dried over anhydrous sodium sulfate, filtered, and evaporated. Purification by silica gel chromatography afforded the corresponding 2-cycloalkyl-1-(4-hydroxy-phenyl)-ethanone.

Utilizing the foregoing experimental procedure the following compounds were prepared:

2-cyclohexyl-1-(4-hydroxy-phenyl)-ethanone: 70% yield using methylene chloride-ethyl acetate (50:1) as the chromatography eluant. ¹H 500 MHz NMR(CDCl₃) ppm(δ): 1-2.0 (m, 11H), 2.96 (d, 1H), 5.6 (bs, 1H), 6.92 (d, 2H), and 7.95 (d, 2H).

2-cyclopentyl-1-(4-hydroxy-phenyl)-ethanone: 74% yield using methylene chloride-ethyl acetate (50:1) as the chromatography eluant. ¹H 500 MHz NMR(CDCl₃) ppm(δ): 1.2-1.92 (m, 10H), 2.4 (m, 1H), 2.96 (d, 1H), 5.6 (bs, 1H), 6.91 (d, 2H), and 7.95 (d, 2H).

### EXAMPLE 5

### Preparation of Isopropyl-4-hydroxy-benzophenone

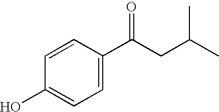

To a mixture of isovaleric acid (1.4 mL, 13.0 mmol) and phenol (1.0253 g, 10.9 mmol) was added BF₃OEt₂ (15 mL) under nitrogen. The resulting mixture was heated to 80° C. for approximately 3.5 h. The reaction was poured into ice/2 N HCl and extracted with EtOAc. The organic extract was washed with brine, dried over Na₂SO₄, and concentrated in vacuo to give a yellow residue. The final product was isolated as a pale yellow oil after silica gel chromatography with 30% EtOAc/hexane as the eluant. Upon standing at ambient temperature, the oil solidified to give a white solid. ¹H 500 MHz NMR(CDCl₃) ppm(δ): 1.01 (d, 6H), 2.27 (m, 1H), 2.81 (d, 2H), 6.99 (d, 2H), 7.93 (d, 2H).

### EXAMPLE 6

### Preparation of 4-pyridyl-4-hydroxy-benzophenone

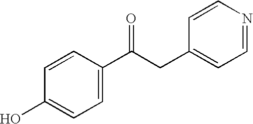

A dried flask equipped with a stirrer bar was charged with a 2.5 M solution of nBuLi in hexane (18 mL, 45.0 mmol) and cooled to 0° C. under N₂. A solution of diisopropylamine (6.4 mL, 45.7 mmol) in distilled THF (20 mL) was added slowly. After stirring for 25 min., a solution of 4-picoline (2.0 mL, 21.4 mmol) in distilled THF (8 mL) was added to the reaction. The resulting red solution was stirred for 25 min. before removing the ice bath. A solution of cyanophenol (2.5670 g, 21.4 mmol) in distilled THF (20 mL) was added via a dropping funnel over 30 min. Upon addition of the phenol, the reaction became a thick slurry with oiling out of a red/brown tar. Further addition of THF did not alleviate the difficulty in stirring. The reaction stood at ambient temperature for 16 h, and was poured into a mixture of ice/sat. NH₄Cl/EtOAc. The intermediate enamine precipitated from the mixture as an insoluble yellow solid and was collected by vacuum filtration. The solid was redissolved in 2 N HCl. The EtOAc layer from the filtrate was also collected and extracted with 2 N HCl/ice. The acidic aqueous extract was combined with the enamine solution in 2 N HCl and stirred at ambient temperature for 16 h. The acidic solution was washed with EtOAc, cooled to 0° C., and neutralized to pH7 with sat. NaHCO₃. The desired product precipitated from the solution as a yellow solid and was collected, washed with cold water, and dried in vacuo. ¹H 500 MHz NMR(d-acetone) ppm(δ): 4.37 (s, 2H), 6.97 (d, 2H), 7.31 (d, 2H), 8.01 (d, 2H), 8.52 (bs, 2H).

### EXAMPLE 7

### Preparation of 3-pyridyl-4-hydroxy-benzophenone

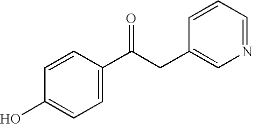

Following the procedure outlined in Example 6 with the exception that 1 equivalent of HMPA in THF was added to the reaction following addition of diisopropylamine, the 3-pyridyl-4-hydroxy-benzophenone was prepared from 3-picoline. The work-up differed slightly in that hydrolysis with 2 N HCl was unnecessary. Instead, the reaction was simply partitioned between ice/sat. NH₄Cl and EtOAc. The organic layer was washed with brine, dried over Na₂SO₄, and concentrated in vacuo. The residue was triturated with CH₂Cl₂ and EtOAc to give the desired product as an orange solid. ¹H 500 MHz NMR(d-acetone) ppm(δ): 4.39 (s, 2H), 6.97 (d, 2H), 7.31 (m, 1H), 7.68 (m, 1 H), 8.01 (d, 2H), 8.43 (m, 1H), 8.52 (m, 1H).

### EXAMPLE 8

### General Preparation of Cycloalkyl-4-triisopropylsilyloxy-benzophenones

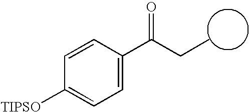

To a stirred solution of the 2-cycloalkyl-1-(4-hydroxy-phenyl)-ethanone, prepared in Example 4, in dry DMF at 0° C. was added 1.3 equivalents of diisopropylethylamine and 1.2 equivalents of triisopropylchlorosilane (TIPSCI). The ice-water bath was removed and the reaction mixture was stirred further until tlc showed the reaction to be complete (1-3 hours) under an inert atmosphere of nitrogen. The reaction mixture was partitioned between ether/2N HCl/ice and the organic phase was separated, washed twice with water, washed with brine, dried over anhydrous sodium sulfate, filtered, and evaporated. Purification by silica gel chromatography afforded the corresponding 2-cycloalkyl-1-(4-triisopropyloxy-phenyl)-ethanone.

Utilizing the foregoing experimental procedure the following compounds were prepared:

2-cyclohexyl-1-(4-triisopropylsilyloxy-phenyl)-ethanone: use methylene chloride-hexanes (1:1) as the chromatography eluant. ¹H 500 MHz NMR(CDCl₃) ppm(δ): 1.13 (d, 18H), 1-1.99 (m, 14H), 2.78 (d, 1H), 6.91 (d, 2H), and 7.89 (d, 2H).

2-cyclopentyl-1-(4-triisopropylsilyloxy-phenyl)-ethanone: use methylene chloride-hexanes(1:1) as the chromatography eluant. ¹H 500 MHz NMR(CDCl₃) ppm(δ):1.12 (d, 18H), 1.2-1.91 (m, 13H), 2.4 (m, 1H), 2.95 (d, 1H), 6.92 (d, 2H), and 7.9 (d, 2H).

### EXAMPLE 9

### General Preparation of Alkyl-4-triisopropylsilyloxy-benzophenones

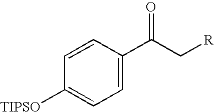

To a solution of the 2-alkyl-1-(4-hydroxy-phenyl)-ethanone, prepared in Examples 3, 6, and 7, in distilled THF was added 1.3 equivalents of 60% NaH in mineral oil at 0° C. under N₂. After the gas evolution ceased, 1.1 equivalents of was added dropwise and the resulting solution stirred for 30 min. The reaction was partitioned between ice/water and EtOAc. The organic layer was washed with brine, dried over Na₂SO₄, and concentrated in vacuo. Purification by silica gel chromatography afforded the corresponding 2-alkyl-1-(4-triisopropylsilyloxy-phenyl)-ethanones.

Utilizing the foregoing experimental procedure the following compounds were prepared:

2-(2-thiophene)-1-(4-triisopropylsilyloxy-phenyl)-ethanone: isolated as an orange/yellow solid using 15% EtOAc/hexane as the chromatography eluant. ¹H 500 MHz NMR(CDCl₃) ppm(δ): 1.14 (d, 18H), 1.30 (m, 3H), 4.42 (s, 2H), and 6.93-7.98 (m, 7 H).

2-(4-pyridyl)-1-(4-triisopropylsilyloxy-phenyl)-ethanone: isolated as a yellow solid using 40% EtOAc/hexane as the chromatography eluant. ¹H 500 MHz NMR(CDCl₃) ppm(δ): 1.14 (d, 18H), 1.30 (m, 3H), 4.28 (s, 2H), 6.97 (d, 2H), 7.35 (m, 1H), 7.69 (m, 1H), 7.97 (d, 2H), and 8.56 (bs, 2H).

2-(3-pyridyl)-1-(4-triisopropylsilyloxy-phenyl)-ethanone: isolated as a yellow solid using 40% EtOAc/hexane as the chromatography eluant. ¹H 500 MHz NMR(CDCl₃) ppm(δ): 1.14 (d, 18H), 1.20 (m, 3H), 4.18 (s, 2H), 6.82 (d, 2H), 7.10 (d, 2H), 7.82 (d, 2H), and 8.43 (d, 2H).

### EXAMPLE 10

### General Bromination Procedure of Alkyl and Cycloalkyl-4-triisopropylselyloxy-benzophenones

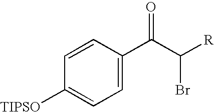

To a stirred solution of the 2-alkyl- and 2-cycloalkyl-1-(4-triisopropylsilyloxy-phenyl)-ethanones, prepared in Examples 8 and 9, in dry THF at 0° C. was added 1.0 equivalent of trimethylammoniumphenyl perbromide. The ice-water bath was removed and the reaction mixture was stirred further for 1 hour under an inert atmosphere of nitrogen. The reaction mixture was partitioned between ethyl acetate/brine/ice/5% sodium thiosulfate/sodium bicarbonate and the organic phase was separated, washed with brine, dried over anhydrous sodium sulfate, filtered, and evaporated. Purification by silica-gel chromatography afforded the corresponding 2-cycloalkyl-2-bromo-1-(4-triisopropylsilyloxy-phenyl)-ethanone.

Utilizing the foregoing experimental procedure the following compounds were prepared:

2-cyclohexyl-2-bromo-1-(4-triisopropylsilyloxy-phenyl)-ethanone: use methylene chloride-hexanes(1:1) as the chromatography eluant. ¹H 500 MHz NMR(CDCl₃) ppm(δ): 1.14 (d, 18H), 0.98-2.27 (m, 15H), 4.91 (d, 1H), 6.94 (d, 2H), and 7.94 (d, 2H).

2-cyclopentyl-2-bromo-1-(4-triisopropylsilyloxy-phenyl)-ethanone: use methylene chloride-hexanes(1:1) as the chromatography eluant. ¹H 50 0MHz NMR(CDCl₃) ppm(δ): 1.13 (d, 18H), 1.1-2.2 (m, 11H), 2.8 (m, 1H), 4.98 (d, 1H), 6.94 (d, 2H), and 7.96 (d, 2H).

2-(2-thiophene)-2-bromo-1-(4-triisopropylsilyloxy-phenyl)-ethanone: stirred at 0° C. for 40 min.; isolated as a dark brown oil and used in the next reaction without purification.

¹H 500 MHz NMR(CDCl₃) ppm(δ): 1.13 (d, 18H), 1.30 (m, 3H), 6.73 (s, 1H), 6.97 (d, 2H), 7.00 (m, 1H), 7.30 (m, 1H), 7.49 (d, 1H), and 8.00 (d, 2H).

2-(4-pyridyl)-2-bromo-1-(4-triisopropylsilyloxy-phenyl)-ethanone: added 2 equivalents of trimethylammoniumphenyl perbromide and stirred at 0° C. for 1 h; isolated as an orange/yellow oil and used in the next reaction without purification. ¹H 500 MHz NMR(CDCl₃) ppm(δ):1.03 (d, 18H), 1.21 (m, 3H), 6.21 (s, 1H), 6.98 (d, 2H), 7.40 (d, 2H), 7.90 (d, 2H), and 8.57 (d, 2H).

2-(3-pyridyl)-2-bromo-1-(4-triisopropylsilyloxy-phenyl)-ethanone: added 2 equivalents of trimethylammoniumphenyl perbromide and stirred at 0° C. for 3 h; isolated as an orange/yellow oil and used in the next reaction without purification. ¹H 500 MHz NMR(CDCl₃) ppm(δ): 1.13 (d, 18H), 1.30 (m, 3H), 6.30 (s, 1H), 6.98 (d, 2H), and 7.39-8.75 (m, 6H).

### EXAMPLE 11

### Preparation of 2-isopropyl-2-bromo-1-(4-hydroxyphenyl)-ethanone

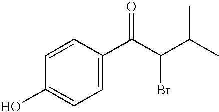

Following the procedure outlined in Example 10 and using the product obtained from Example 5, 2-isopropyl-2-bromo-1-(4-hydroxyphenyl)-ethanone was isolated as a yellow oil and used in the next reaction without purification. ¹H 500 MHz NMR(CDCl₃) ppm(δ): 1.01 (d, 3H), 1.21 (d, 3H), 2.46 (m, 1H), 4.93 (d, 1H), 6.96 (d, 2H), and 7.96 (d, 2H).

### EXAMPLE 12

### General Preparation of Bromoketones

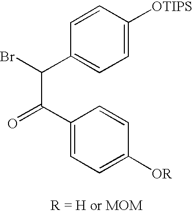

Step A

To a stirred solution of 3.0 g (13.2 mmole) of dry desoxybenzoin (freshly azeotroped with toluene) in 25 mL of DMF at 0° C. was added 5.7 mL (5.7 mmole) of neat diisopropylethylamine. To this stirred solution was added slowly 1.25 mL (19.73 mmole) of chloromethylmethylether (MOMCl). The ice-water bath was removed and the mixture was stirred further under an atmosphere of nitrogen for 18 hours. The mixture was then poured into a saturated NaHCO₃ solution, extracted with EtOAc, and the extract washed with water, and dried over anhydrous MgSO₄. After evaporation of the solvent, the residue was purified by silica gel chromatography (EtOAc/hexane=1:1) to provide the product, as a solid. ¹H NMR (400 MHz, CDCl₃) δ (ppm): 8.0 (d, 2H), 7.19(d, 2H), 7.10 (d, 2H), 6.8 (d, 2H), 5.23 (s, 2H), 4.8 (s, 1H), 4.2 (s, 2H), 3.5 (s, 3H).

Step B

To a stirred solution of the product obtained from Step A (423 mg, 1.55 mmole) and imidazole (211 mg, 3.1 mmole) in 20 mL of dry DMF at 0° C. was added triisopropylsilyl chloride (3.1 mmole) and the reaction mixture was allowed to warm to room temperature and stirred further for 2-3 hours. The reaction was quenched by the addition of aqueous NaHCO₃ solution and extracted with EtOAc. The organic layer was washed with brine and dried with MgSO₄. Chromatography (10% EtOAc/hexane) yielded the desired product. ¹H NMR (400 MHz, CDCl₃) δ (ppm): 8.0 (d, 2H), 7.12 (d, 2H), 7.08 (d, 2H). 6.82 (d, 2H), 5.21 (s, 2H), 4.18 (s, 2H), 3.5 (s, 3H), 1.24 (m, 3H), 1.1 (d, 18H).

Step C

To a mixture of the compound from Step B (0.5 g, 1.16 mmole) in 100 mL of anhydrous THF was added 0.39 g (1.16 mmole) of trimethylphenylammonium perbromide (PTAB) at 0° C. The ice-water bath was removed, and the mixture was stirred further for one hour. The solution was then filtered and washed with water and brine and dried over MgSO₄. Removal of the solvent afforded the mixture of bromo-ketones (MOM group was partially removed), which was used without further purification due to their instability toward chromatography.

Bromoketone with MOM group: ¹H NMR (400 MHz, CDCl₃) δ (ppm): 8.0 (d, 2H), 7.4 (d, 2H), 6.88 (d, 2H), 6.86 (d, 2H), 6.36 (s, 1H), 1.24 (m, 3H), 1.1 (d, 18H);

Bromoketone without MOM group: ¹H NMR (400 MHz, CDCl₃) δ (ppm): 7.94 (d, 2H), 7.4 (d, 2H), 6.88 (d, 2H), 6.86 (d, 2H), 6.36 (s, 1H), 1.24 (m, 3H), 1.1 (d, 18H).

### EXAMPLE 13

### Preparation of

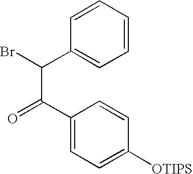

The required bromoketone was prepared using the procedure in Example 12 (Step C). ¹H NMR (400 MHz, CDCl₃) δ (ppm) 7.94 (d, 2H), 7.56 (m, 2H), 7.38 (m, 3H), 6.9 (d, 2H), 6.36 (s, 2H), 1.28 (m, 3H), 1.1 (d, 18H).

### EXAMPLE 14

### Preparation of

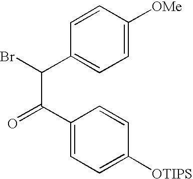

The required bromoketone was prepared using the procedure in Example 12 (Step C). ¹H NMR (400 MHz, CDCl₃) δ (ppm) 7.9 (d, 2H), 7.5 (d, 2H), 6.9 (d & d, 4H), 6.4 (s, 1H), 3.8 (s, 3H), 1.28 (m, 3H), 1.1 (d, 18H).

### EXAMPLE 15

### Preparation of

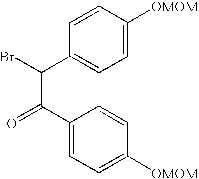

Step A

To a stirred solution of a mixture of the 0.1 g (0.37 mmole) mono phenolic compound from Step A in Example 12 and diisopropylethylamine (0.13 mL, 2 eq) in 5 mL of DMF at room temperature was added slowly neat MOMCl (0.05 mL, 2 eq), and the mixture was heated at 85° C. under N₂ for three hours. The mixture was then poured into a saturated NaHCO₃ solution, extracted with EtOAc, washed with water, and dried over MgSO₄. After evaporation of the solvent, the residue was purified by silica gel chromatography (EtOAc/Hexane=1:1) to provide the pure bis-protected MOM product, as a solid. ¹H NMR (400 MHz, CDCl₃) δ (ppm): 8.0 (d, 2H), 7.19(d, 2H), 7.10 (d, 2H), 7.02 (d, 2H), 5.23 (s, 2H), 5.2 (s, 2H), 4.2 (s, 2H), 3.5 (two s, 6H).

Step B

The product of Step A was treated with bromine to give the bromoketone. 1H NMR (400 MHz, CDCl₃) δ (ppm): 8.0 (d, 2H), 7.45(d, 2H), 7.10 (two d, 4H), 6.4 (s, 1H), 5.23 (two s, 4H), 3.5 (two s, 6H).

### EXAMPLE 16

### General Preparation of

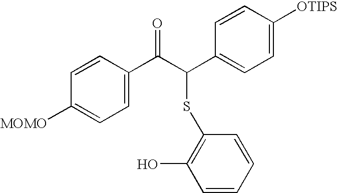

To a stirred, freshly prepared solution of 2-thiophenol (0.2 g, 1.6 mmole) and Et₃N (0.34 mL, 2 eq) in 15 mL DMF at 0° C. was slowly added a solution of 0.627 g (1.232 mmole) of bromoketone (prepared from Step C in Example 12) in 13 mL of DMF. The reaction mixture was stirred for three hours at room temperature and was then partitioned between saturated NaHCO₃ and EtOAc, the layers were separated, and the aqueous layer was extracted again with EtOAc. The combined organic layers were dried (Na₂SO₄), filtered, and evaporated in vacuo. The resulting oil was purified by flash chromatography (EtOAc/Hex=1/4) to provide the desired product as an oil. ¹H NMR (400 MHz, acetone-d₆) δ (ppm): 8.0 (d, 2H), 7.2-6.6 (m, 8H), 6.8 (d, 2H), 6.2 (s, 1H), 5.24 (s, 2H), 3.4 (s, 3H), 1.22 (m, 3H), 1.1 (d, 18H); MS m/z 575 (M⁺+23).

### EXAMPLE 17

### Cyclization of Coupled Product

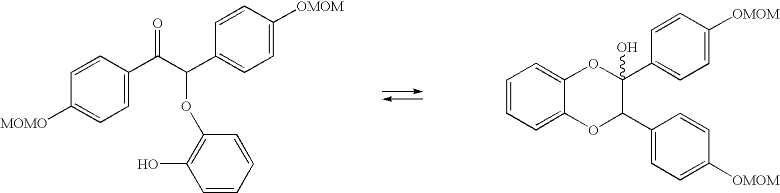

Following the procedure outlined in Example 16, 1,2-dihydroxybenzene and the bromide of Example 15 was converted to the product which was purified by silica gel chromatography using EtOAc/hexane (1/4) as eluant. MS m/z 448 (M⁺+23).

### EXAMPLE 18

### Preparation of

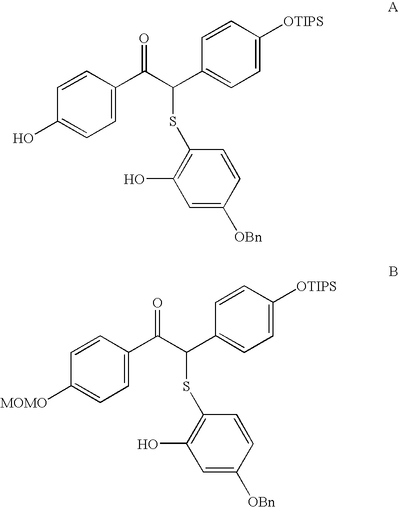

Following the procedure outlined in Example 16 and using 0.83 g (3.6 mmole) of 4-benzyloxy-thiophenol, prepared from Example 1, product A and product B were obtained after silica gel chromatography using EtOAc/hexane (1/5) as eluant.

A: ¹H NMR (400 MHz, acetone-d₆) δ (ppm): 8.15 (s, 1H), 7.8 (d, 2H), 7.4 (m, 5H), 6.98 (d, 2H), 6.98 (d, 1H), 6.75 (d & d, 4H), 6.0 (s, 1H), 5.62 (s, 1H), 5.0 (s, 2H), 1.22 (m, 3H), 1.15 (d, 18H).

B: ¹H NMR (400 MHz, acetone-d₆) δ (ppm): 8.0 (d, 2H), 7.5 (m, 5H), 7.18 (d, 2H), 7.04 (d, 2H), 6.96 (d, 1H), 6.8 (d, 2H), 6.56 (d, 1H), 6.32 (dd, 1H), 6.1 (s, 1H), 5.25 (s, 2H), 5.09 (s, 1H), 3.4 (s, 3H), 1.22 (m, 3H), 1.1 (d, 18H).

### EXAMPLE 19

### Preparation of

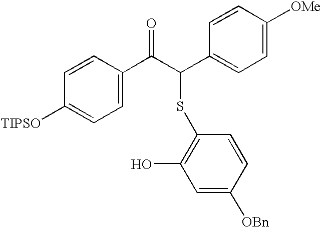

Following the procedure outlined in Example 16 and using 1.1 g (2.3 mmole) of the bromoketone from Example 14, the desired product was obtained after silica gel chromatography using EtOAc/hexane (1/5) as eluant. ¹H NMR (400 MHz, acetone-d₆) δ (ppm): 8.46 (br s, 1H), 7.98 (d, 2H), 7.48-7.3 (m, 5H), 7.24 (d, 2H), 7.4 (d, 1H), 6.92 (d, 2H), 6.82 (d, 2H), 6.56 (d, 1H), 6.38 (dd, 1H), 6.1 (s, 1H), 5.04 (s, 2H), 3.72 (s, 3H), 1.25 (m, 3H), 1.1 (d, 18H).

### EXAMPLE 20

### Preparation of

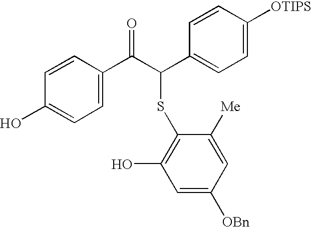

Following the procedure outlined in Example 16 and using 0.74 g (1.5 mmole) of the bromoketone from Example 12 (Step C), the desired product was obtained after silica gel chromatography using EtOAc/hexane (1/5) as eluant. ¹H NMR (400 MHz, acetone-d₆) δ (ppm): 7.92 (d, 2H), 7.46-7.1 (m, 5H), 7.18 (d, 2H), 6.84 (d, 2H), 6.78 (d, 2H), 6.42 (d, 1H), 6.36 (d, 1H), 5.98 (s, 1H), 5.02 (s, 2H), 2.2 (s, 3H), 1.22 (m, 3H), 1.1 (d, 18H).

### EXAMPLE 21

### Preparation of

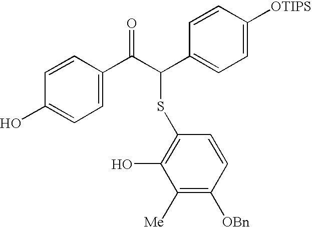

Following the procedure outlined in Example 16 and using 0.8 g (1.57 mmole) of the bromoketone from Example 12 (Step C) with the thiophenol derivative prepared from Example 1, the desired product was obtained after silica gel chromatography using EtOAc/hexane (1/5) as eluant. ¹H NMR (400 MHz, acetone-d₆) δ (ppm): 7.9 (d, 2H), 7.5-7.3 (m, 5H), 7.12 (d, 2H), 6.9 (d, 1H), 6.84 (d, 2H), 6.79 (d, 2H), 6.4 (d, 1H), 6.0 (s 1H), 5.1 (s, 2H), 2.1 (s, 3H), 1.25 (m, 3H), 1.1 (d, 18H).

### EXAMPLE 22

### Preparation of

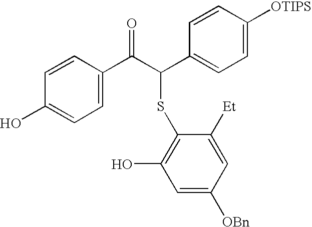

Following the procedure outlined in Example 16 and using 0.56 g (1.1 mmole) of the bromoketone from Example 12 (Step C) with 0.19 g (0.73 mmole) of thiophenol derivative prepared from Example 1, the desired product was obtained after silica gel chromatography using EtOAc/hexane (1/5) as eluant. ¹H NMR (400 MHz, acetone-d₆) δ (ppm): 7.9 (d, 2H), 7.48-7.3 (m, 5H), 7.16 (d, 2H), 6.84 (d, 2H), 6.78 (d, 2H), 6.42 (d, 1H), 6.38 (d, 1H), 5.96 (s, 1H), 5.1 (s, 2H), 2.6 (q, 2H), 1.22 (m, 3H), 1.1 (d, 18H), 1.1 (t, 3H).

### EXAMPLE 23

### Preparation of

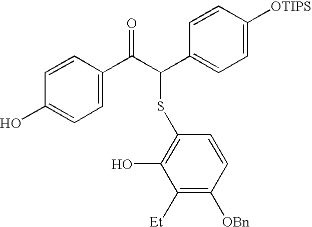

Following the procedure outlined in Example 16 and using 2.04 g (4.33 mmole) of the bromoketone from Example 12 (Step C) with the thiophenol derivative prepared from Example 1, the desired product was obtained after silica gel chromatography using EtOAc/hexane (1/5) as eluant. ¹H NMR (400 MHz, acetone-d₆) δ (ppm): 7.9 (d, 2H), 7.5-7.3 (m, 5H), 7.12 (d, 2H), 6.92 (d, 1H), 6.84 (d, 2H), 6.78 (d, 2H), 6.42 (d, 1H), 6.0 (s, 1H), 5.1 (s, 2H), 2.7 (q, 2H), 1.24 (m, 3H), 1.1 (d & t, 21H).

### EXAMPLE 24

### Preparation of

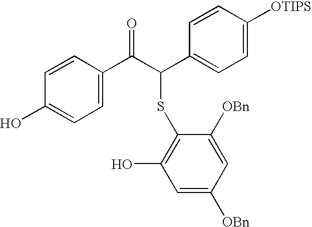

Following the procedure outlined in Example 16 and using 2.0 g (4.33 mmole) of the bromoketone from Example 12 (Step C) with the thiophenol derivative prepared from Example 1, the desired product was obtained after silica gel chromatography using EtOAc/hexane (1/5) as eluant. ¹H NMR (400 MHz, acetone-d₆) δ (ppm): 7.8 (d, 2H), 7.62 (d, 2H), 7.48-7.3 (m, 8H), 7.12 (d, 2H), 6.8 (d, 2H), 6.76 (2H, d), 6.28 (d, 1H), 6.18 (d, 1H), 6.0 (s, 1H), 5.24 (s, 2H), 5.05 (s, 2H), 1.22 (m, 3H), 1.1 (d, 18H).

### EXAMPLE 25

### Preparation of

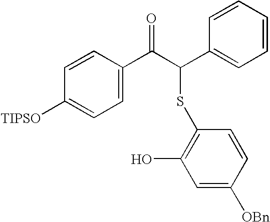

Following the procedure outlined in Example 16 and using 1.6 g (3.51 mmole) of the bromoketone from Example 13 with the thiophenol derivative prepared from Example 1, the desired product was obtained after silica gel chromatography using EtOAc/hexane (1/5) as eluant. ¹H NMR (400 MHz, acetone-d₆) δ (ppm): 8.0 (d, 2H), 7.5-7.2 (m, 10H), 7.0 (d, 1H), 6.92 (d, 2H), 6.54 (d, 1H), 6.35 (dd, 1H), 6.12 (s, 1H), 5.06 (s, 2H), 1.22 (m, 3H), 1.1 (d, 18H).

### EXAMPLE 26

### Preparation of

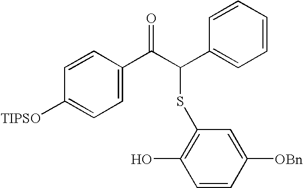

Following the procedure outlined in Example 16 and using 2.6 g (5.82 mmole) of the bromoketone from Example 13 with the thiophenol derivative prepared from Example 1, the desired product was obtained after silica gel chromatography using EtOAc/hexane (1/5) as eluant. ¹H NMR (400 MHz, acetone-d₆) δ (ppm): 8.0 (d, 2H), 7.4-7.2 (m, 10H), 6.94 (d, 2H), 6.84-6.74 (m, 3H), 6.24 (s, 1H), 4.85 (s, 2H), 1.23 (m, 3H), 1.1 (d, 18H).

### EXAMPLE 27

### Preparation of

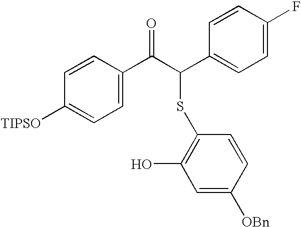

Following the procedure outlined in Example 16 and using the bromoketone from Example 12 (Step C) with the thiophenol derivative prepared from Example 1, the desired product was obtained after silica gel chromatography using EtOAc/hexane (1/5) as the eluant. ¹H NMR (400 MHz, acetone-d₆) δ (ppm): 8.0 (d, 2H), 7.4-7.2 (m, 7H), 7.0 (m, 5H), 6.54 (d, 1H), 6.28 (dd, 1H), 6.14 (s, 1H), 5.08 (s, 2H), 1.23 (m, 3H), 1.1 (d, 18H).

### EXAMPLE 28

### Preparation of

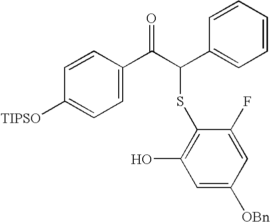

Following the procedure outlined in Example 16 and using the bromoketone from Example 13 with the appropriate thiophenol derivative prepared from Example 1, the desired product was obtained after silica gel chromatography using EtOAc/hexane (1/5) as the eluant. ¹H NMR (500 MHz, CDCl₃) δ (ppm) 8.28 (s, 1H), 7.82 (d, 2H), 7.40 (m, 5H), 7.22 (m, 5H), 6.80 (d, 2H), 6.40 (d, 1H), 6.21 (dd, 1H), 5.80 (s, 1H), 5.00 (s, 2H), 1.24 (m, 3H), 1.10 (d, 18H).

### EXAMPLE 29

### Preparation of

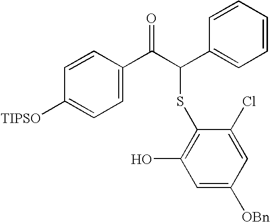

Following the procedure outlined in Example 16 and using the bromoketone from Example 13 with the appropriate thiophenol derivative prepared from Example 1, the desired product was obtained after SiO₂ using EtOAc/hexane (1/5) as eluant. ¹H NMR (500 MHz, CDCl₃) δ (ppm) 8.19(s, 1H), 7.82(d, 2H), 7.40(m, 5H), 7.24(m, 5H), 6.80(d, 2H), 6.64(d, 1H), 6.44(d, 1H), 5.84(s, 1H), 5.00(s, 2H), 1.23(m, 3H), 1.10(m, 18H).

### EXAMPLE 30

### Preparation of

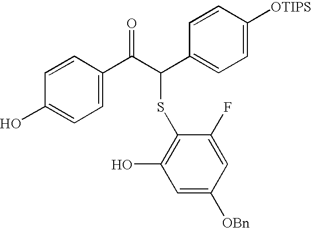

Following the procedure outlined in Example 16 and using the bromoketone from Example 12 with the thiophenol derivative prepared from Example 1, the desired product was obtained after silica gel chromatography using EtOAc/hexane (1/5) as eluant. ¹H NMR (500 MHz, CDCl₃) δ (ppm): 8.20 (s, 1H), 7.81 (d, 2H), 7.40 (m, 5H), 7.02 (d, 2H), 6.75 (d, 4H), 6.36 (d, 1H), 6.20 (dd, 1H), 5.78 (s, 1H), 4.95 (s, 2H), 1.23 (m, 3H), 1.10 (m, 18H).

### EXAMPLE 31

### Preparation of

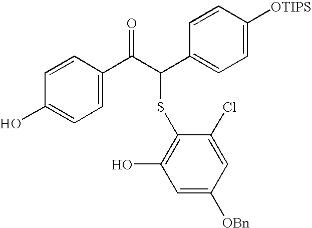

Following the procedure outlined in Example 16 and using the bromoketone from Example 12 with the thiophenol derivative prepared from Example 1, the desired product was obtained after silica gel chromatography using EtOAc/hexane (1/5) as eluant. ¹H NMR (500 MHz, CDCl₃) δ (ppm): 8.24 (s, 1H), 7.80 (d, 2H), 7.40 (m, 5H), 7.10 (d, 2H), 6.78 (d, 4H), 6.62 (d, 1H), 6.42 (d, 1H), 5.84 (s, 1H), 4.98 (s, 2H), 1.23 (m, 3H), 1.10 (m, 18H); MS m/z 650 (M⁺+1).

### EXAMPLE 32

### Preparation of

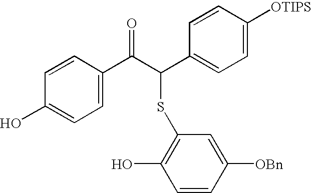

Following the procedure outlined in Example 16 and using the bromoketone from Example 12 with the thiophenol derivative prepared from Example 1, the desired product was obtained after silica gel chromatography using EtOAc/hexane (1/5) as eluant. ¹H NMR (500 MHz, acetone-d₆) δ (ppm): 7.95 (d, 2H), 7.40 (m, 5H), 7.20 (d, 2H), 6.80 (m, 7H), 6.20 (s, 1H), 4.85 (s, 2H), 1.23 (m, 3H), 1.10 (m, 18H); MS m/z 616 (M⁺+1).

### EXAMPLE 33

### Preparation of

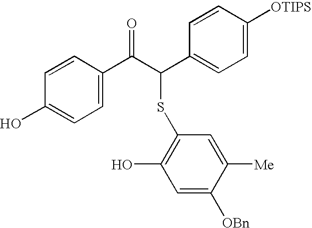

Following the procedure outlined in Example 169 and using the bromoketone from Example 12 with the thiophenol derivative prepared from Example 1, the desired product was obtained after silica gel chromatography using EtOAc/hexane (1/5) as eluant. ¹H NMR (500 MHz, CDCl₃) δ (ppm): 7.82 (d, 2H), 7.40 (m, 5H), 7.05 (d, 2H), 6.95 (s, 1H), 6.80 (d, 4H), 6.52 (s, 1H), 5.64 (s, 1H), 5.00 (s, 2H), 1.23 (m, 3H), 1.10 (m, 18H); MS m/z 629 (M⁺+1).

### EXAMPLE 34

### Preparation of

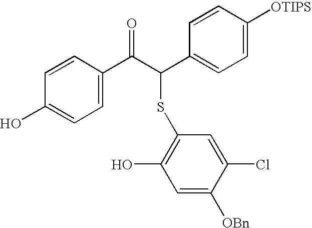

Following the procedure outlined in Example 16 and using the bromoketone from Example 12 with the thiophenol derivative prepared from Example 1, the desired product was obtained after silica gel chromatography using EtOAc/hexane (1/5) as eluant. ¹H NMR (500 MHz, CDCl₃) δ (ppm: 8.24 (s, 1H), 7.80 (d, 2H), 7.40 (m, 5H), 7.10 (d, 2H), 6.78 (d, 2H), 6.76 (d, 2H), 6.64 (d, 2H), 6.45 (d, 2H), 5.86 (s, 1H), 4.98 (s, 2H), 1.23 (m, 3H), 1.10 (m, 18H); MS m/z 650 (M⁺+1).

### EXAMPLE 35

### Preparation of

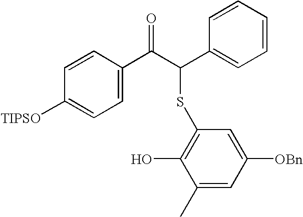

Following the procedure outlined in Example 16 and using the bromoketone from Example 12 with the thiophenol derivative prepared from Example 1, the desired product was obtained after silica gel chromatography using EtOAc/hexane (1/5) as eluant. ¹H NMR (500 MHz, CDCl₃) δ (ppm): 7.82 (d, 2H), 7.40 (m, 5H), 7.24 (m, 3H), 7.20 (d, 2H), 6.82 (d, 2H), 6.80 (d, 2H), 6.58 (d, 2H), 5.65 (s, 1H), 4.80 (d, 2H), 2.22 (s, 3H), 1.23 (m, 3H), 1.10 (m, 18H).

### EXAMPLE 36

### Preparation of

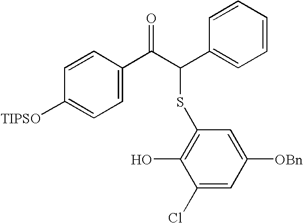

Following the procedure outlined in Example 16 and the bromoketone from Example 13 with the thiophenol derivative prepared from Example 1, the desired product was obtained after silica gel chromatography using EtOAc/hexane (1/5) as eluant. ¹H NMR (500 MHz, CDCl₃) δ (ppm): 7.98 (s, 1H), 7.82 (d, 2H), 7.40 (m, 5H), 7.25 (m, 3H), 7.20 (d, 2H), 7.00 (d, 1H), 6.80 (d, 2H), 6.60 (d, 1H), 5.78 (s, 1H), 4.78 (d, 2H), 1.23 (m, 3H), 1.10 (m, 18H).

### EXAMPLE 37

### Preparation of

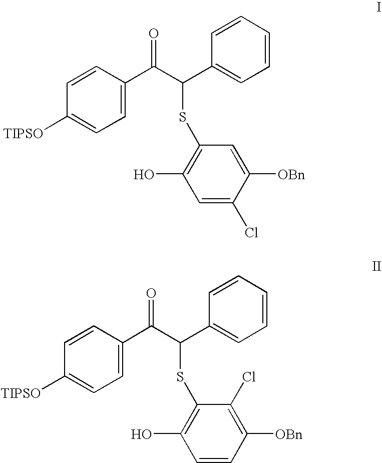

Following the procedure outlined in Example 16 and using the bromoketone from Example 13 with the mixture of the two thiophenol derivatives prepared from Example 1, the two desired products I and II were obtained after silica gel chromatography using EtOAc/hexane (1/5) as eluant.

I: ¹H NMR (500 MHz, CDCl₃) δ (ppm): 7.80 (d, 2H), 7.40 (m, 5H), 7.25 (m, 3H), 7.16 (d, 2H), 7.04 (s, 1H), 6.80 (d, 2H), 6.60 (s, 1H), 5.78 (s, 1H), 4.80 (d, 2H), 1.23 (m, 3H), 1.10 (m, 18H).

II: ¹H NMR (500 MHz, CDCl₃) δ (ppm): 7.80 (d, 2H), 7.65 (s, 1H), 7.44 (d, 1H), 7.40 (m, 5H), 7.25 (m, 5H), 6.96 (d, 1H), 6.80 (m, 3H), 6.00 (s, 1H), 5.15 (s, 2H), 1.23 (m, 3H), 1.10 (m, 18H).

### EXAMPLE 38

### Preparation of

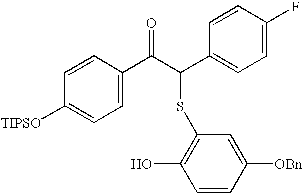

Following the procedure outlined in Example 16 and using the bromoketone from Example 12 with the thiophenol derivative prepared from Example 1, the desired product was obtained after silica gel chromatography using EtOAc/hexane (1/5) as eluant. ¹H NMR (500 MHz, CDCl₃) δ (ppm): 7.80 (d, 2H), 7.40 (m, 5H), 7.14 (m, 2H), 6.96 (m, 2H), 6.84 (m, 2H), 6.82 (d, 2H), 6.70 (d, 1H), 5.68 (s, 1H), 4.86 d, 2H), 1.23 (m, 3H), 1.10 (m, 18H).

### EXAMPLE 39

### General Preparation of

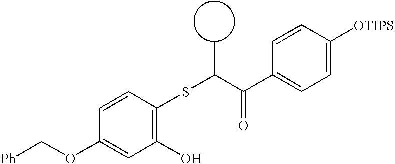

Utilizing the bromides prepared in the Example 10 and the appropriate mercaptan prepared in Example 1 and employing the procedure outlined in Example 16 the following compounds were prepared:

Cyclohexyl derivative: use methylene chloride/hexanes (3:1) as the chromatography eluant. ¹H 500 MHz NMR(CDCl₃) ppm(δ): 1.12 (d, 18H), 1.11-2.34 (m, 15H), 4.19 (d, 1H), 5.0 (s, 2H), 6.44 (dd, 1H), 6.54 (d, 1H), 6.86 (m, 3H), 7.25-7.72 (m, 7H).

Cyclopentyl derivative: use methylene chloride/hexanes (2:1) as the chromatography eluant. ¹H 500 MHz NMR(CDCl₃) ppm(δ): 1.12 (d, 18H), 1.28-2.49 (m, 12H), 4.18 (d, 1H), 5.0 (s, 2H), 6.45-7.77 (m, 12H).

### EXAMPLE 40

### Preparation of

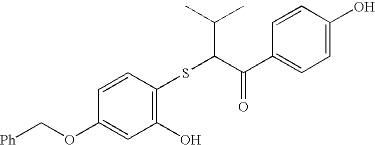

Utilizing the bromide prepared in Example 11 and the appropriate mercaptan prepared in Example 1 and employing the procedure outlined in Example 9, the desired product was obtained as a yellow oil in 77% yield after silica gel chromatography with 30% EtOAc/hexane as the eluant. ¹H 500 MHz NMR (CDCl₃) ppm(δ): 1.00 (d, 3H), 1.21 (d, 3 H), 2.30 (m, 1H), 4.13 (d, 1H), 4.99 (s, 2H), 6.41-7.72 (m, 12H), 8.02 (bs, 1H), 8.80 (bs, 1H); MS m/z 409 (M⁺).

### EXAMPLE 41

### General Preparation of

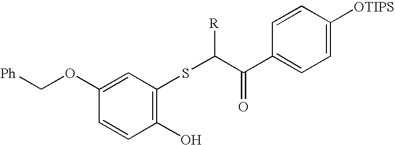

Utilizing the bromides prepared in Example 10 and the appropriate mercaptan prepared in Example 1 and employing the procedure outlined in Example 16 the following compounds were prepared:

Cyclohexyl derivative: use methylene chloride/hexanes (3:1) as the chromatography eluant. ¹H 500 MHz NMR(CDCl₃) ppm(δ): 1.12 (d, 18H), 1.11-2.3 (m, 15H), 4.24 (d, 1H), 4.89 (m, 2H), 6.8-7.6 (m, 12H).

Cyclopentyl derivative: use methylene chloride/hexanes (2:1) as the chromatography eluant. ¹H 500 MHz NMR(CDCl₃) ppm(δ): 1.12 (d, 18H), 1.26-2.12 (m, 11H), 2.5 (m, 1H), 4.24 (d, 1H), 4.9 (m, 2H), 6.8-7.69 (m, 12H).

4-Pyridyl derivative: isolated as a yellow oil using 30% EtOAc/hexane as the chromatography eluant. ¹H 500 MHz NMR(CDCl₃) ppm(δ): 1.12 (d, 18H), 1.28 (m, 3H), 4.84 (q, 2 H), 4.88 (s, 1H), 5.63 (s, 1H), and 6.69-8.50 (m, 16H).

3-Pyridyl derivative: isolated as a yellow oil using 30% EtOAc/hexane as the chromatography eluant. ¹H 500 MHz NMR(CDCl₃) ppm(δ): 1.12 (d, 18H), 1.28 (m, 3H), 4.84 (q, 2H), 4.90 (s, 1H), 5.79 (s, 1H), and 6.70-8.50 (m, 16H).

### EXAMPLE 42

### Preparation of

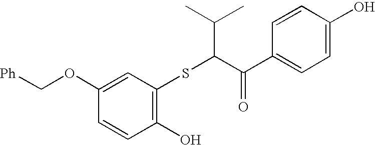

Utilizing the bromide prepared in Example 11 and the appropriate mercaptan prepared in Example 1 and employing the procedure outlined in Example 16, the desired product was obtained as a yellow oil after silica gel chromatography with 30% EtOAc/hexane as the eluant. ¹H 500 MHz NMR(CDCl₃) ppm(δ): 1.02 (d, 3H), 1.21 (d, 3 H), 2.34 (m, 1H), 4.13 (d, 1H), 4.90 (q, 2H), 6.25 (bs, 1H), 6.79-7.70 (m, 12H).

### EXAMPLE 43

### Preparation of

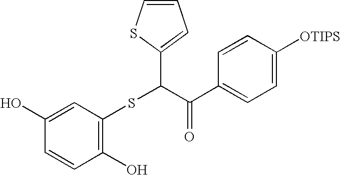

Utilizing the appropriate bromide prepared in Example 10 and the mercaptoquinol [prepared according to the method of Burton, etal, J. Chem. Soc., 1952, 2193] and employing the procedure outlined in Example 16, the desired product was obtained as an orange/red oil after silica-gel chromatography with 30% EtOAc/hexane as the eluant. ¹H 500 MHz NMR(CDCl₃) ppm(δ): 1.10 (d, 18H), 1.27 (m, 3H), 6.00 (s, 1H), and 6.76-7.89 (m, 10H); MS m/z 515 (M⁺).

### EXAMPLE 44

### Preparation of

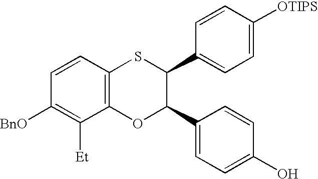

To a flask charged with 0.1 g (0.16 mmole) of thio-ketone generated in Example 22 in dichloromethane (ca 0.04M) was slowly added trifluoroacetic acid(TFA) (2×0.062 mL, 10 eq) under an N₂ atmosphere at room temperature. To the stirred reaction mixture was slowly added triethylsilane (2×0.05 mL, 4 eq) and the resulting mixture until starting material was consumed (approximately 5-6 hours, as monitored by TLC). The reaction mixture was poured into saturated NaHCO₃/ice water, stirred 10 minutes, and extracted with dichloromethane. The organic extract was washed with brine (2×50 mL), dried with Na₂SO₄, and concentrated in vacuo to afford a light yellow oil. Purification via flash chromatography (EtOAc/Hex=1:5) provided the desired compound as an oil. ¹H NMR (400 MHz, CDCl₃) δ (ppm): 7.44 (m, 5H), 6.98 (d, 1H), 6.90 (d, 2H), 6.75 (d, 2H), 6.68 (d, 2H), 6.65 (d, 1H), 6.63 (d, 2H), 5.51 (d, J=2.3 Hz, 1H), 5.10 (s, 2H), 4.74 (brs, 1H), 4.32 (d, J=2.3 Hz, 1H), 2.77 (qd, 2H), 1.22 (m, 3H), 1.08 (d, 18H), 1.1 (m, 3H); MS m/z 628.5 (M⁺+1).

### EXAMPLE 45

### Preparation of

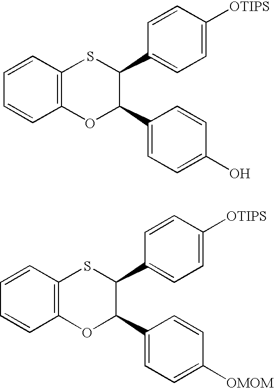

Utilizing the procedure from Example 44, the desired dihydrobenzoxathiin without MOM protection was isolated after purification by silica gel chromatography with 10% EtOAc/hexane. ¹H NMR (400 MHz, CDCl₃) δ (ppm): 7.2-6.98 (m, 4H), 6.85 (d, 2H), 6.78 (d, 2H), 6.66 (two d, 4H), 5.5 (d, J=2.2 Hz, 1H), 4.8 (s, 1H), 4.33 (d, J=2.1 Hz, 1H), 1.22 (m, 3H), 1.1 (d, 18H); MS m/z 515 (M⁺+23).

The other dihydrobenzooxathiin with MOM protection was also isolated. ¹H NMR (400 MHz, CDCl₃) δ (ppm): 7.2-6.6 (m, 8H), 6.78 (d, 2H), 6.66 (d, 2H), 5.5 (d, J=2.4 Hz, 1H), 5.14 (s, 2H), 4.35 (d, J=2.1 Hz, 1H), 3.48 (s, 3H), 1.22 (m, 3H), 1.1 (d, 18H).

### EXAMPLE 46

### Preparation of

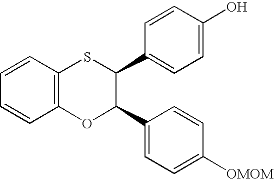

Utilizing the procedure from Example 71 (Step C), the dihydrobenzoxathiin generated from Example 45, was desilylated to give the product. ¹H NMR (400 MHz, CDCl₃) δ (ppm): 7.2-6.96 (m, 4H), 6.92 (two d, 4H), 6.82 (d, 2H), 6.6 (d, 2H), 5.52 (d, J=2.2 Hz, 1H), 5.16 (s, 2H), 4.68 (br s, 1H), 4.38 (d, J=2.2 Hz, 1H), 3.48 (s, 3H).

### EXAMPLE 47

### Preparation of

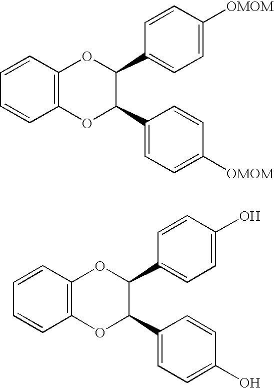

The ketone generated in Example 17 was converted to the desired product following the procedure described in Example 44 with the exception that 5 equivalents of TFA and 2 equivalents of Et₃SiH was necessary to drive the reaction to completion. The MOM group was removed with mild acid treatment (2N-HCl, 75° C.) to give the desired product. ¹H NMR (400 MHz, CDCl₃) δ (ppm): 7.0 (m, 4H), 6.85 (d, 2H), 6.65 (d, 2H), 5.38 (s, 2H); MS m/z 343 (M⁺+23).

### EXAMPLE 48

### Preparation of

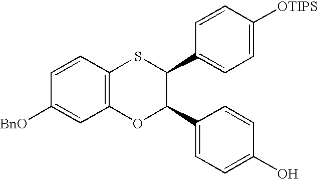

The ketone generated in Example 18 was converted to the dihydrobenzoxathiin utilizing the procedure from Example 44 with the exception that 20 equivalents of TFA and 15 equivalents of Et₃SiH were necessary to drive the reaction to completion. The desired product was isolated after purification by silica gel chromatography using 10% EtOAc/hexane as eluant. ¹H NMR (400 MHz, CDCl₃) δ (ppm): 7.5-7.34 (m, 5H), 7.08 (d, 1H), 6.84 (d, 2H), 6.76 (d, 2H), 6.7 (dd, 1H), 6.67 (d, 1H), 6.68 (two d, 4H), 5.5 (d, J=2.2 Hz, 1H), 5.04 (br q, 2H), 4.68 (s, 1H), 4.3 (d, J=2.2 Hz, 1H), 1.22 (m, 3H), 1.1 (d, 18H); MS m/z 515 (M⁺+23).

### EXAMPLE 49

### Preparation of

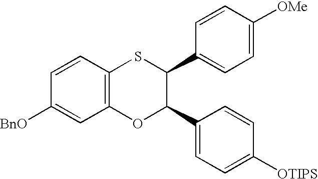

The ketone generated in Example 19 was converted to the dihydrobenzoxathiin utilizing the procedure from Example 44 with the exception that the reaction was run at −10° C. for 48 hours in the presence of 20 equivalents of TFA and 2 equivalents of Et₃SiH. The desired product [with 20% recovered starting material] was isolated after purification by silica gel chromatography using 10% EtOAc/hexane as eluant. ¹H NMR (400 MHz, CDCl₃) δ (ppm): 7.5-7.3 (m, 5H), 7.1-6.6 (m, 11H), 5.54 (d, J=1.9 Hz, 1H), 5.06 (dd, 2H), 4.32 (d, 1H), 3.74 (s, 3H), 1.22 (m, 3H), 1.1 (d, 18H).

### EXAMPLE 50

### Preparation of

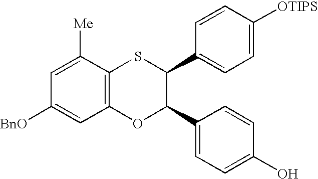

Following the procedure outlined in Example 44 and using the ketone derivative in Example 20, the desired product was obtained after purification by silica gel chromatography using 5% EtOAc/hexane as eluant. ¹H NMR (400 MHz, CDCl₃) δ (ppm): 7.46-7.32 (m, 5H), 6.84 (d, 2H), 6.78 (d, 2H), 6.66 (two d, 4H), 6.62 (d, 1H), 6.57 (d, 1H), 5.3 (d, J=2.2 Hz, 1H), 4.35 (d, 1H), 2.28 (s, 3H), 1.22 (m, 3H), 1.1 (d, 18H).

### EXAMPLE 51

### Preparation of

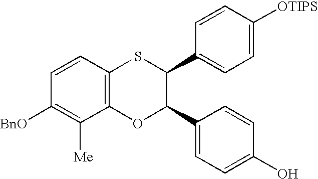

Following the procedure outlined in Example 44 and using the ketone derivative from Example 21, the desired product was obtained after purification by silica gel chromatography using 5% EtOAc/hexane as eluant. ¹H NMR (400 MHz, CDCl₃) δ (ppm): 7.5-7.3 (m, 5H), 6.98 (d, 1H), 6.9 (d, 1H), 6.76 (d, 2H), 6.6 (m, 5H), 5.51 (d, J=2.2 Hz, 1H), 5.1 (s, 2H), 4.8 (s, 1H), 4.32 (d, 1H), 2.4 (s, 3H), 1.22 (m, 3H), 1.1 (d, 18H).

### EXAMPLE 52

### Preparation of

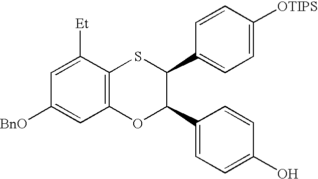

Following the procedure outlined in Example 44 and using the ketone derivative from Example 22, the desired product was obtained after purification by silica gel chromatography using 5% EtOAc/Hexane as eluant. ¹H NMR (400 MHz, CDCl₃) δ (ppm): 7.5-7.3 (m, 5H), 6.85 (d, 2H), 6.78 (d, 2H), 6.66 (m, 5H), 6.56 (d, 1H), 5.48 (d, J=2.0 Hz, 1H), 5.04 (br q, 2H), 4.74 (br s, 1H), 4.34 (d, J=2.0 Hz, 1H), 2.64 (q, 2H), 1.3 (t, 3H), 1.24 (m, 3H), 1.1 (d, 18H).

### EXAMPLE 53

### Preparation of

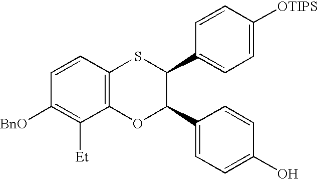

Following the procedure outlined in Example 44 and using the ketone derivative from Example 23, the desired product was obtained after purification by silica gel chromatography using 5% EtOAc/hexane as eluant. ¹H NMR (400 MHz, CDCl₃) δ (ppm: 7.5-7.3 (m, 5H), 6.98 (d, 1H), 6.9 (d, 2H), 6.74 (d, 2H), 6.7-6.6 (three d, 5H), 5.5 (d, J=2.3 Hz, 1H), 5.1 (s, 2H), 4.74 (br s, 1H), 4.32 (d, J=2.4 Hz, 1H), 2.79 (m, 2H), 1.22 (m, 3H), 1.1 (d & t, 2H); MS n/z 628.5 (M⁺+1).

### EXAMPLE 54

### Preparation of

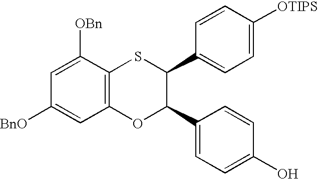

Following the procedure outlined in Example 44 and using the ketone derivative from Example 24, the desired product was obtained after purification by silica gel chromatography using 5% EtOAc/hexane as eluant. ¹H NMR (400 MHz, CDCl₃) δ (ppm): 7.5-7.3 (m, 10H), 6.84 (d, 2H), 6.78 (d, 2H), 6.66 (two d, 4H), 6.38 (s, 2H), 5.48 (d, J=2.1 Hz, 1H), 5.14 (s, 2H), 5.0 (q, 2H), 4.76 (br s, 1H), 4.32 (d, J=2.1 Hz, 1H), 1.22 (m, 3H), 1.1 (d, 18H).

### EXAMPLE 55

### Preparation of

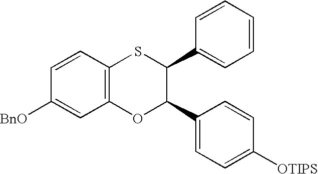

Following the procedure outlined in Example 44 and using the ketone derivative obtained from Example 25, the desired product was obtained after purification by silica gel chromatography using 5% EtOAc/hexane as eluant. ¹H NMR (400 MHz, CDCl₃) δ (ppm): 7.5-7.32 (m, 5H), 7.2-7.1 (m, 4H), 6.9-6.82 (m, 4H), 6.76-6.7 (m, 4H), 5.56 (d, 1H), 5.06 (br q, 2H), 4.36 (d, 1H), 1.22 (m, 3H), 1.1 (d, 18H).

### EXAMPLE 56

### Preparation of

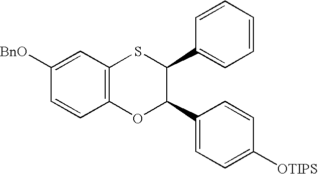

Following the procedure outlined in Example 44, with the exception that the reaction was run at 0° C. for three hours, and using 1.7 g (2.83 mmole) of the ketone derivative obtained from Example 26, the desired product was obtained after purification by silica gel chromatography using 5% EtOAc/hexane as eluant. ¹H NMR (400 MHz, CDCl₃) δ (ppm): 7.5-7.34 (m, 5H), 7.2-7.1 (m, 3H), 6.94 (d, 1H), 6.9-6.82 (m, 5H), 6.4 (m, 3H), 5.48 (d, J=1.9 Hz, 1H), 5.05 (s, 2H), 4.36 (d, J=1.9 Hz, 1H), 1.22 (m, 3H), 1.1 (d, 18H).

### EXAMPLE 57

### Preparation of

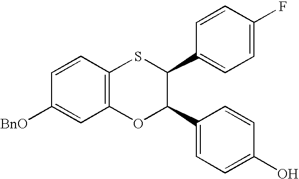

Following the procedure outlined in Example 44 and using the ketone derivative obtained from Example 27, the desired product was obtained, which was subsequently desilylated using the procedure described in Example 71 (Step C). The desired product was obtained as an oil after purification by silica gel chromatography using 15% EtOAc/hexane as eluant. ¹H NMR (400 MHz, CDCl₃) δ (ppm): 7.5-7.32 (m, 5H), 7.09 (d, 1H), 6.9-6.8 (m, 6H), 6.73-6.7 (m, 4H), 5.52 (d, 1H), 5.04 (br q, 2H), 4.34 (d, 1H), 1.22 (m, 3H), 1.1 (d, 18H).

### EXAMPLE 58

### Preparation of

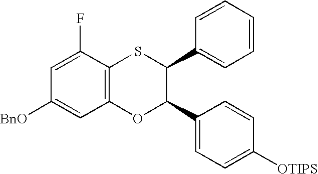

Following the procedure outlined in Example 44 and using the ketone derivative from Example 28, the desired product was obtained after purification by silica gel chromatography using 5% EtOAc/hexane as eluant. ¹H NMR (500 MHz, CDCl₃) δ (ppm): 7.5-7.3 (m, 5H), 7.22-7.10 (m, 3H), 6.90-6.80 (2d, 4H), 6.75 (d, 2H), 6.55 (d, 2H), 5.55 (d, J=2.1 Hz, 1H), 5.05 (d, 2H), 4.40 (d, J=2.1 Hz, 1H), 1.22 (m, 3H), 1.1 (d, 18H).

### EXAMPLE 59

### Preparation of

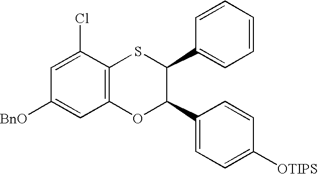

Following the procedure outlined in Example 44 and using the ketone derivative from Example 29, the desired product was obtained after purification by silica gel chromatography using 5% EtOAc/hexane as eluant. ¹H NMR (500 MHz, CDCl₃) δ (ppm): 7.5-7.3 (m, 5H), 7.22-7.10 (m, 3H), 6.90-6.80 (2d, 4H), 6.73 (d, 2H), 6.64 (d, 2H), 5.50 (d, J=2.1 Hz, 1H), 5.05 (d, 2H), 4.43 (d, J=2.2 Hz, 1H), 1.23 (m, 3H), 1.10 (d, 18H).

### EXAMPLE 60

### Preparation of

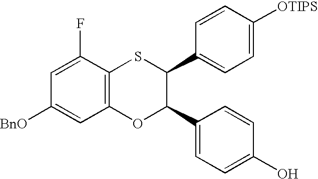

Following the procedure outlined in Example 44 and using the ketone derivative from Example 30, the desired product was obtained after purification by silica gel chromatography using 5% EtOAc/hexane as eluant. ¹H NMR (500 MHz, CDCl₃) δ (ppm): 7.5-7.3 (m, 5H), 6.82 (d, 2H), 6.68 (d, 2H), 6.64 (d, 2H), 6.62 (d, 2H), 6.46 (d, 2H), 5.44 (d, J=1.9 Hz, 1H), 5.02 (d, 2H), 4.30 (d, J=2.0 Hz, 1H), 1.22 (m, 3H), 1.10 (d, 18H); MS m/z 618 (M⁺+1).

### EXAMPLE 61

### Preparation of

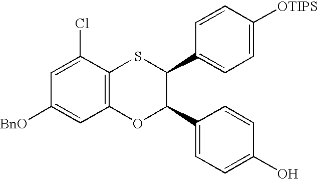

Following the procedure outlined in Example 44 and using the ketone derivative from Example 31, the desired product was obtained after purification by silica gel chromatography using 5% EtOAc/hexane as eluant. ¹H NMR (400 MHz, CDCl₃) δ (ppm: 7.5-7.3 (m, 5H), 6.86 (d, 1H), 6.82 (d, 2H), 6.76 (d, 2H), 6.70 (d, 1H), 6.67(d, 2H), 6.65(d, 2H), 5.44 (d, J=2.0 Hz, 1H), 5.04 (s, 2H), 4.38 (d, J=1.9 Hz, 1H), 1.23 (m, 3H), 1.10 (d, 18H); MS m/z 634 (M⁺+1).

### EXAMPLE 62

### Preparation of

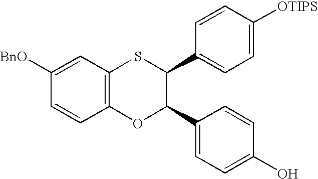

Following the procedure outlined in. Example 44 and using the ketone derivative from Example 32, the desired product was obtained after purification by silica gel chromatography using 5% EtOAc/hexane as eluant. ¹H NMR (500 MHz, CDCl₃) δ (ppm): 7.5-7.3 (m, 5H), 6.94 (d, 1H),6.85 (d, 2H), 6.80 (d, 2H), 6.74 (dd, 2H), 6.65(m, 4H), 5.43 (d, J=2.1 Hz, 1H), 5.05 (d, 2H), 4.30 (d, J=2.1 Hz, 1H), 1.23 (m, 3H), 1.10 (d, 18H).

### EXAMPLE 63

### Preparation of

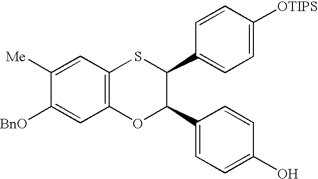

Following the procedure outlined in Example 44 and using the ketone derivative from Example 33, the desired product was obtained after purification by silica gel chromatography using 5% EtOAc/hexane as eluant. ¹H NMR (500 MHz, CDCl₃) δ (ppm): 7.5-7.3 (m, 5H), 6.88 (s, 1H), 6.84 (d, 2H), 6.82 (d, 2H), 6.70 (d, 2H), 6.68 (d, 2H), 6.66 (s, 1H), 5.50 (d, 1H), 5.05 (s, 2H), 4.43 (d, 1H), 2.35 (s, 3H), 1.23 (m, 3H), 1.10 (d, 18H).

### EXAMPLE 64

### Preparation of

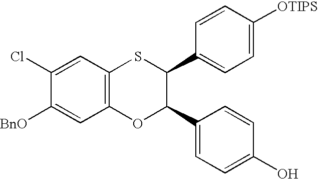

Following the procedure outlined in Example 44 and using the ketone derivative from Example 34, the desired product was obtained after purification by silica gel chromatography using 5% EtOAc/hexane as eluant. ¹H NMR (500 MHz, CDCl₃) δ (ppm): 7.5-7.3 (m, 5H), 7.24 (s, 1H), 7.20 (s, 1H), 6.82 (d, 2H), 6.68 (d, 2H), 6.64 (m, 4H), 5.44 (d, J=2.0 Hz, 1H), 5.05 (d, 2H), 4.28 (d, J=2.3 Hz, 1H), 1.23 (m, 3H), 1.10 (d, 18H).

### EXAMPLE 65

### Preparation of

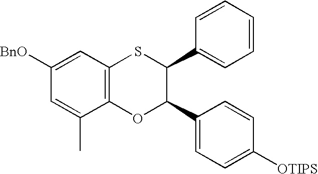

Following the procedure outlined in Example 44 and using the ketone derivative from Example 35, the desired product was obtained after purification silica gel chromatography using 5% EtOAc/hexane as eluant. ¹H NMR (500 MHz, CDCl₃) δ (ppm): 7.5-7.3 (m, 5H), 7.05-7.20 (m, 4H), 6.90 (d, 2H), 6.88 (d, 2H), 6.78 (d, 2H), 6.70 (d, 1H), 6.65 (d, 1H), 5.30 (d, J=1.8 Hz, 1H), 5.05 (d, 2H), 4.20 (d, J=2.3 Hz, 1H), 1.23 (m, 3H), 1.10 (d, 18H).

### EXAMPLE 66

### Preparation of

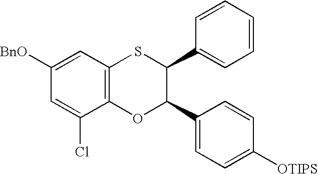

Following the procedure outlined in Example 44 and using the ketone derivative from Example 36, the desired product was obtained after purification by silica gel chromatography using 5% EtOAc/hexane as eluant. ¹H NMR (500 MHz, CDCl₃) δ (ppm): 7.5-7.3 (m, 5H), 7.05-7.20 (m, 2H), 7.10 (m, 2H), 6.98 (d, 2H), 6.88 (m, 2H), 6.80 (m, 1H), 6.60 (d, 1H), 5.56 (d, J=1.8 Hz, 1H), 5.05 (d, 2H), 4.44 (d, J=2.3 Hz, 1H), 1.23 (m, 3H), 1.10 (d, 18H).

### EXAMPLE 67

### Preparation of

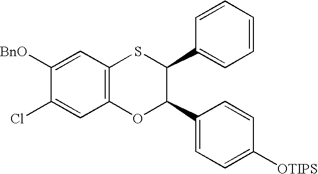

Following the procedure outlined in Example 44 and using the ketone derivative from Example 37(I), the desired product was obtained after purification by silica gel chromatography using 5% EtOAc/hexane as eluant. ¹H NMR (500 MHz, CDCl₃) δ (ppm): 7.55 (d, 2H), 7.45 (t, 2H), 7.35 (t, 1H), 7.20 (d, 1H), 7.15 (m, 3H), 6.88 (d, 2H), 6.84 (d, 3H), 6.78 (d, 2H), 5.46 (d, J=2.1 Hz, 1H), 5.15 (s, 2H), 4.39 (d, J=2.1 Hz, 1H), 1.23 (m, 3H), 1.10 (d, 18H).

### EXAMPLE 68

### Preparation of

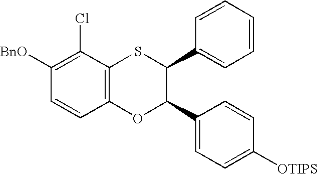

Following the procedure outlined in 44 and using the ketone derivative from Example 37(II), the desired product was obtained after purification by silica gel chromatography using 5% EtOAc/hexane as eluant. ¹H NMR (500 MHz, CDCl₃) δ (ppm): 7.55 (d, 2H), 7.45 (t, 2H), 7.35 (t, 1H), 7.20 (d, 1H), 7.15 (t, 2H), 6.80-6.90 (m, 4H), 6.78 (d, 2H), 6.76 (d, 2H), 5.42 (d, J=2.1 Hz, 1H), 5.18 (s, 2H), 4.42 (d, J=2.1 Hz, 1H), 1.23 (m, 3H), 1.10 (d, 18H).

### EXAMPLE 69

### Preparation of

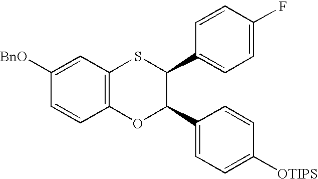

Following the procedure outlined in Example 44 and using the ketone derivative from Example 38, the desired product was obtained after purification by silica gel chromatography using 5% EtOAc/hexane as eluant. ¹H NMR (500 MHz, CDCl₃) δ (ppm): 7.36-7.50 (m, 5H), 6.96 (d, 2H), 6.80-6.90 (m, 4H), 6.70-6.78 (m, 5H), 5.42 (d, J=2.1 Hz, 1H), 5.18 (s, 2H), 4.38 (d, J=2.1 Hz, 1H), 1.23 (m, 3H), 1.10 (d, 18H).

### EXAMPLE 70

### Chiral Separation of

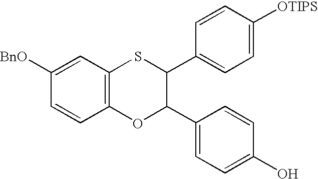

Each enantiomer of the racemic-dihydrobenzoxathiin, obtained from Example 62, was obtained via chiral chromatography using a Chiralpak AD column, with 30% isopropanol in hexane as the eluant.

The fast moving isomer: [α]D=+18.44°(c=0.725, MeOH).

The slow moving isomer: [α]D=−18.85°(c=0.74, MeOH).

### EXAMPLE 71

### General Preparation of THIINS

### Preparation of

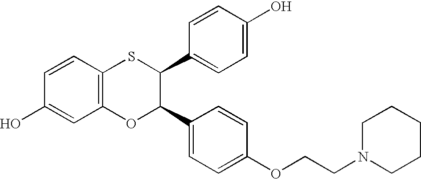

Step A

To a stirred solution of a mixture of dihydrobenzoxathiin (60 mg, 0.1 mmole), obtained from Example 48 (which was dried by the azeotropic method prior to use), triphenylphosphine (157 mg, 0.6 mmole), and 1-piperidineethanol (0.08 mL, 0.6 mmole) in 4 mL of anhydrous THF at 0° C. was added dropwise 0.118 mL (0.6 mmole) of diisopropyl azodicarboxylate (DIAD) over 0.2 hours. The resulting pale yellow solution was stirred at room temperature for 2-3 hours. The volatile components were removed in vacuo and the residue purified by flash chromatography (EtOAc/hexane=1:5, followed by 2-3% MeOH/dichloromethane) to give desired product. ¹H NMR (400 MHz, CDCl₃) δ (ppm): 7.5-7.34 (m, 5H), 7.08 (d, 1H), 6.86 (d, 2H), 6.78-6.64 (m, 8H), 5.5 (d, 1H), 5.01 (br q, 2H), 4.3 (d, 1H), 4.2 (t, 2H), 2.75 (t, 2H), 2.5 (br s, 4H), 1.6 (m, 4H), 1.48 (m, 2H), 1.22 (m, 3H), 1.1 (d, 18H); MS m/z 712.4 (M⁺+1).

Step B

To a stirred solution of the adduct (71 mg, 0.098 mmole), generated in Step A, in 2 mL of EtOH/EtOAc/H₂O (7:2:1) was added 13 mg (1.2 eq) of palladium black and ammonium formate (62 mg, 10 eq).The resulting mixture was heated at 80° C. and monitored by TLC. After 3 hours, the reaction mixture was cooled to room temperature, filtered through a pad of Celite to remove the catalyst, and the filtrate was partitioned between water and EtOAc. The organic phase was separated, dried over MgSO₄ and concentrated in vacuo to give desired product. ¹H NMR (400 MHz, CDCl₃) δ (ppm): 7.01 (d, 1H), 6.8 (d, 2H), 6.75 (d, 2H), 6.66 (two d, 4H), 6.54 (dd, 1H), 6.5 (d, 1H), 5.45 (d, J=2.3 Hz, 1H), 4.28 (d, J=2.3 Hz, 1H), 4.08 (t, 2H), 2.8 (t, 2H), 2.6 (br s, 4H), 1.68 (m, 4H), 1.5 (m, 2H), 1.22 (m, 3H), 1.1 (d, 18H).

Step C

To a stirred solution of a mixture of the debenzylated product generated in Step B and HOAc (10 eq) in mL of THF was added a solution of tetrabutylammonium fluoride (3 eq) in THF at room temperature. The resulting solution was allowed to stir for two hours at room temperature and then poured into saturated aqueous NaHCO₃ and extracted with EtOAc. The organic layer was washed with brine, dried over MgSO₄, filtered, and evaporated. Purification by silica gel chromatography using 5-7% MeOH in methylene chloride as eluant afforded the desired product. ¹H NMR (400 MHz, CD₃OD) δ (ppm): 6.95 (d, 2H), 6.92 (d, 1H), 6.78 (d, 2H), 6.71 (d, 2H), 6.48 (d, 2H), 6.47 (d, 1H), 6.44 (dd, 1H), 5.47 (d, J=2.1 Hz, 1H), 4.37 (d, J=2.1 Hz, 1H), 4.1 (t, 2H), 2.85 (t, 2H), 2.65 (br s, 4H), 1.66 (m, 4H), 1.5 (m, 2H).

### EXAMPLE 72

### Preparation of

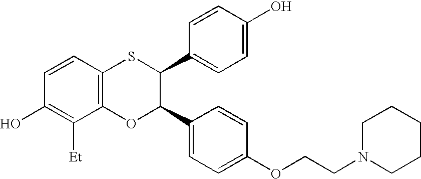

Step A

Using the procedure described in Example 71 (Step A), the dihydrobenzoxathiin obtained from Example 53 was coupled with 1-piperidineethanol. After purification by silica gel chromatography, using 3% MeOH/CH₂Cl₂ as eluant, the desired adduct was obtained. ¹H NMR (400 MHz, CDCl₃) 8 (ppm): 6.98 (d, 1H), 6.92 (d, 2H), 6.74 (two d, 4H), 6.65 (d, 1H), 6.62 (d, 2H), 5.5 (d, 1H), 5.1 (s, 2H), 4.31 (d, 1H), 4.09 (m, 2H), 2.75 (t, 2H), 2.55 (m, 2H), 2.5 (m, 4H), 1.6 (m, 4H), 1.45 (m, 2H), 1.22 (m, 3H), 1.1 (m, 21H).

Step B

The adduct generated in Step A was debenzylated using the procedure described in Example 71 (Step B) to give the desired product. ¹H NMR (400 MHz, CDCl₃) δ (ppm): 6.92 (d, 1H), 6.89 (d, 2H), 6.72 (d & d, 4H), 6.62 (d, 2H), 6.5 (d, 1H), 5.5 (d, J=2.2 Hz, 1H), 4.3 (d, J=2.2 Hz, 1H), 4.1 (m, 2H), 2.8 (t, 2H), 2.68 (m, 2H), 2.58 (br s, 4H), 1.64 (m, 4H), 1.48 (m, 2H), 1.2 (m, 3H), 1.09 (d & m, 21H).

Step C

The debenzylated product from Step B was desilylated using the procedure described in Example 71 (Step C). The desired product was obtained as a white solid. ¹H NMR (400 MHz, CD₃OD) δ (ppm): 7.0 (d, 2H), 6.79 (d, 2H), 6.76 (d, 1H), 6.71 (d, 2H), 6.47 (d, 3H), 5.46 (d, J=2.2 Hz, 1H), 4.38 (d, 1H), 4.08 (t, 2H), 2.8 (t, 2H), 2.5 (m, 2H), 2.6 (m, 4H), 1.62 (m, 4H), 1.5 (m, 2H), 1.1 (t, 3H); MS m/z 493.2 (M⁺+1).

### EXAMPLE 73

### Preparation of

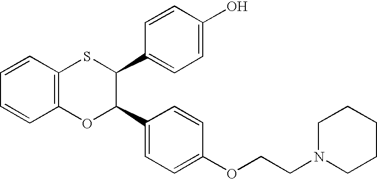

Step A

The dihydrobenzoxathiin obtained from Example 45 was coupled with 1-piperidineethanol using the procedure described in Example 71 (Step A). After purification by silica gel chromatography using 3% MeOH/CH₂Cl₂ as eluant, the desired adduct was obtained. 1H NMR (400 MHz, CDCl₃) δ (ppm): 7.14-6.92 (m, 4H), 6.8 (d, 2H), 6.76 (d, 2H), 6.72 (d, 2H), 6.64 (d, 2H), 5.48 (d, J=2.2 Hz, 1H), 4.34 (d, J=2.1 Hz, 1H), 4.1 (m, 2H), 2.85 (m, 2H), 2.6 (m, 4H), 1.65 (m, 4H), 1.5 (m, 2H), 1.22 (m, 3H), 1.1 (d, 18H).

Step B

The adduct from Step A was desilylated using the procedure described in Example 71 (Step C). The desired product was obtained as a white solid. ¹H NMR (400 MHz, CD₃OD) δ (ppm): 7.14-6.92 (m, 4H), 6.06 (d, 2H), 6.78 (d, 2H), 6.72 (d, 2H), 6.48 (d, 2H), 5.48 (d, J=2.1 Hz, 1H), 4.44 (d, 1H), 4.1 (t, 2H), 2.78 (t, 2H), 2.58 (br s, 4H), 1.64 (m, 4H), 1.5 (m, 2H); MS m/z 450.2 (M⁺+1).

### EXAMPLE 74

### Preparation of

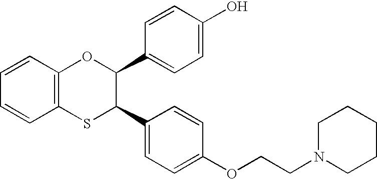

Step A

The dihydrobenzoxathiin obtained from Example 46 was coupled with 1-piperidineethanol using the procedure described in Example 71 (Step A). After purification by silica gel chromatography with 3% MeOH/CH₂Cl₂, the desired adduct was obtained as an oil. ¹H NMR (400 MHz, CDCl₃) δ (ppm): 7.14-6.94 (m, 4H), 6.96 (d, 2H), 6.84 (two d, 4H), 6.66 (d, 2H), 5.5 (d, J=2.1 Hz, 1H), 5.12 (s, 2H), 4.5 (d, J=2.1 Hz, 1H), 4.04 (t, 2H), 3.42 (s, 3H), 2.75 (t, 2H), 2.55 (br s, 4H), 1.6 (m, 4H), 1.48 (m, 2H); MS m/z 495.2 (M⁺+1).

Step B

The adduct (10 mg, 0.02 mmole) from Step A was deprotected with TFA (10 eq) and MeOH (6 eq) in CH₂Cl₂ at room temperature to afford the desired product. ¹H NMR (400 MHz, CD₃OD) δ (ppm): 7.14-6.92 (m, 4H), 6.84 (two d, 4H), 6.66 (d, 2H), 6.6 (d, 2H), 5.45 (d, J=2.2 Hz, 1H), 4.45 (d, J=2.2 Hz, 1H), 4.05 (t, 2H), 2.8 (t, 2H), 2.6 (br s, 4H), 1.6 (m, 4H), 1.5 (m, 2H) ; MS m/z 450.2 (M⁺+1).

### EXAMPLE 75

### Preparation of

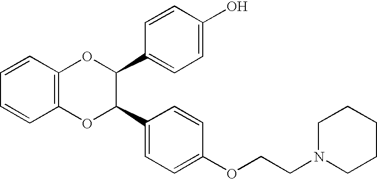

The dioxane derivative obtained from Example 47 was coupled with 1-piperidineethanol using the procedure described in Example 71 (Step A) to give the product. ¹H NMR (400 MHz, CD₃OD) δ (ppm): 7.04 (d, 2H), 6.98-6.84 (m, 4H), 6.82 (d, 2H), 6.74 (d, 1H), 6.63 (d, 2H), 6.56 (d, 2H), 5.36 (d, 1H), 5.33 (d, J=3.0 Hz, 1H), 4.02 (m, 2H), 2.8 (m, 2H), 2.6 (br s, 4H), 1.62 (m, 4H), 1.5 (m, 2H); MS m/z 432 (M⁺).

### EXAMPLE 76

### Preparation of

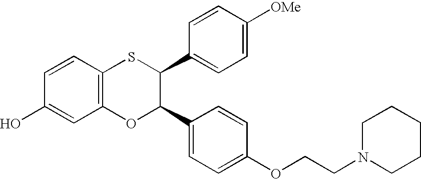

Step A

The dihydrobenzoxathiin generated from Example 49 was desilylated using the procedure described in Example 71 (Step C). The desired product was obtained as a white solid. ¹H NMR (400 MHz, CDCl₃) δ (ppm): 7.5-7.3 (m, 5H), 7.2 (d, 1H), 6.9 (d, 2H), 6.88 (d, 2H), 6.68 (m, 6H), 5.53 (d, J=2.2 Hz, 1H), 4.33 (d, J=2.3 Hz, 1H), 3.75 (s, 3H).

Step B

The desilylated product obtained from Step A was coupled-with 1-piperidineethanol using the procedure described in Example 71 (Step A). After purification by silica gel chromatography with 3% MeOH/CH₂Cl₂, the desired adduct was obtained. ¹H NMR (400 MHz, CDCl₃) δ (ppm): 7.5-7.3 (m, 5H), 7.08 (d, 1H), 6.9 (d, 2H), 6.84 (d, 2H), 6.76 (d, 2H), 6.66 (m, 4H), 5.52 (d, 1H), 5.03 (s, 2H), 4.32 (d, 1H), 4.06 (t, 2H), 3.75 (s, 3H), 2.75 (t, 2H), 2.5 (br s, 4H), 1.6 (m, 4H), 1.45 (m, 2H).

Step C

The adduct generated in Step B was debenzylated using the procedure described in Example 71 (Step B) to give the product. ¹H NMR (400 MHz, CD₃OD) δ (ppm): 6.96 (d, 2H), 6.92 (d, 1H), 6.82 (d, 2H), 6.78 (d, 2H), 6.63 (d, 2H), 6.48 (dd, 1H), 6.44 (d, 1H), 5.5 (d, J=2.2 Hz, 1H), 4.42 (d, J=2.2 Hz, 1H), 4.08 (t, 2H), 3.68 (s, 3H), 2.78 (t, 2H), 2.59 (br s, 4H), 1.6 (m, 4H), 1.48 (m, 2H); MS m/z 479.4 (M⁺+1).

### EXAMPLE 77

### Preparation of

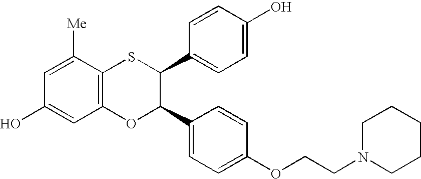

Step A

The dihydrobenzoxathiin obtained from Example 50 was coupled with 1-piperidineethanol using the procedure described in Example 71 (Step A). After purification by silica gel chromatography with 3% MeOH/CH₂Cl₂, the desired adduct was obtained. ¹H NMR (400 MHz, CDCl₃) δ (ppm): 6.83 (d, 2H), 6.75 (d, 2H), 6.69 (d, 2H), 6.62 (d, 2H), 6.5 (d, 1H), 6.48 (d, 1H), 5.42 (br s, 1H), 4.3 (br s, 1H), 4.06 (t, 2H), 2.78 (t, 2H), 2.5 (br s, 4H), 1.6 (m, 4H), 1.44 (m, 2H), 1.22 (m, 3H), 1.1 (d, 18H).

Step B

The adduct generated in Step A was debenzylated using the procedure described in Example 71 (Step B).

Step C

The debenzylated product from Step B was desilylated using the procedure described in Example 71 (Step C). The desired product was obtained as a white solid. ¹H NMR (400 MHz, CD₃OD) δ (ppm): 6.94 (d, 2H), 6.76 (d, 2H), 6.7 (d, 2H), 6.49 (d, 2H), 6.4 (d, 1H), 6.32 (d, 1H), 5.43 (d, J=2.3 Hz, 1H), 4.4 (d, J=2.3 Hz, 1H), 4.08 (t, 2H), 2.8 (t, 2H), 2.6 (br s, 4H), 2.18 (s, 3H), 1.64 (m, 4H), 1.5 (m, 2H); MS m/z 479.2 (M⁺+1).

### EXAMPLE 78

### Preparation of

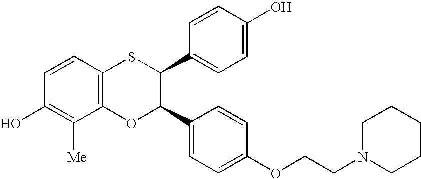

Step A

The dihydrobenzoxathiin obtained from Example 51 was coupled with 1-piperidineethanol using the procedure described in Example 71 (Step A). After purification by silica gel chromatography with 3% MeOH/CH₂Cl₂, the desired adduct was obtained.

Step B

The adduct generated in Step A was debenzylated using the procedure described in Example 71 (Step B). After purification by silica gel chromatography using 5% MeOH/CH₂Cl₂ as the eluant, the desired product was obtained as an oil. ¹H NM (400 MHz, CDCl₃) δ (ppm): 6.9 (d, 2H), 6.89 (d, 1H), 6.73 (m, 4H), 6.62 (d, 2H), 6.52 (d, 1H), 5.5 (d, 1H), 4.3 (d, 1H), 4.1 (br s, 2H), 2.8 (br t, 2H), 2.6 (br s, 4H), 2.2 (s, 3H), 1.6 (m, 4H), 1.5 (m, 2H), 1.22 (m, 3H), 1.1 (d, 18H).

Step C

The debenzylated product from Step B was desilylated using the procedure described in Example 71 (Step C). The desired product was obtained as a white solid. ¹H NMR (400 MHz, CD₃OD) δ (ppm): 7.02 (d, 2H), 6.76 (d, 2H), 6.7 (d, 2H), 6.47 (two d, 3H), 5.48 (d, J=2.3 Hz, 1H), 4.38 (d, J=2.3 Hz, 1H), 4.1 (t, 2H), 2.8 (t, 2H), 2.6 (br s, 4H), 2.1 (s, 3H), 1.6 (m, 4H), 1.5 (m, 2H); MS m/z 479.2 (M⁺+1).

### EXAMPLE 79

### Preparation of

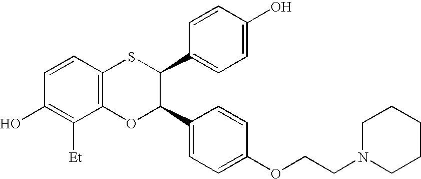

Step A

The dihydrobenzoxathiin obtained from Example 53 was coupled with 1-piperidineethanol using the procedure described in Example 71 (Step A). After purification by silica gel chromatography with 3% MeOH/CH₂Cl₂, the desired adduct was obtained.

Step B

The adduct generated in Step A was debenzylated using the procedure described in Example 71 (Step B).

Step C

The debenzylated product from Step B was desilylated using the procedure described in Example 71 (Step C). The desired product was obtained as a white solid after silical gel chromatography with 5% MeOH/CH₂Cl₂ as eluant. ¹H NMR (400 MHz, CD₃OD) δ (ppm): 6.94 (d, 2H), 6.76 (d, 2H), 6.7 (2H, d), 6.48 (d, 2H), 6.41 (d, 1H), 6.3 (d, 1H), 5.44 (d, J=2.2 Hz, 1H), 4.4 (d, J=2.2 Hz, 1H), 4.08 (t, 2H), 2.8 (t, 2H), 2.62 (br s, 4H), 2.6 (q, 2H), 1.6 (m, 4H), 1.45 (m, 2H), 1.2 (t, 3H); MS m/z 493.2 (M⁺+1).

### EXAMPLE 80

### Preparation of

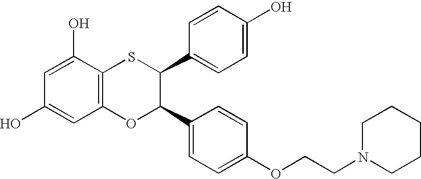

Step A

The dihydrobenzoxathiin obtained from Example 54 was coupled with 1-piperidineethanol using the procedure described in Example 71 (Step A). After purification by silica gel chromatography with 3% MeOH/CH₂Cl₂, the desired adduct was obtained. ¹H NMR (400 MHz, CDCl₃) δ (ppm): 7.5-7.3 (m, 10H), 6.86 (d, 2h), 6.78 (d, 2H), 6.74 (d, 2H), 6.64 (d, 2H), 6.38 (s, 2H), 5.48 (d, 1H), 5.14 (s, 2H), 5.02 (q, 2H), 4.32 (d, 1H), 4.08 (t, 2H), 2.8 (t, 2H), 2.5 (br s, 4H), 1.62 (m, 4H), 1.5 (m, 2H), 1.22 (m, 3H), 1.1 (d, 18H).

Step B

The adduct generated in Step A was debenzylated using the procedure described in Example 71 (Step B). After purification by silica gel chromatography using 5% MeOH/CH₂Cl₂ as eluant, the desired product was obtained as an oil.

Step C

The debenzylated product from Step B was desilylated using the procedure described in Example 71 (Step C). The desired product was obtained as a white solid. ¹H NMR (400 MHz, CD₃OD) δ (ppm): 6.94 (d, 2H), 6.78 (d, 2H), 6.72 (d, 2H), 6.5 (d, 2H), 6.06 (d, 1H), 6.02 (d, 1H), 5.42 (d, J=2.2 Hz, 1H), 4.33 (d, J=2.2 Hz, 1H), 4.09 (t, 2H), 2.8 (t, 2H), 2.6 (br s, 4H), 1.64 (m, 4H), 1.5 (m, 2H); MS m/z 482.2 (M⁺+1).

### EXAMPLE 81

### Preparation of

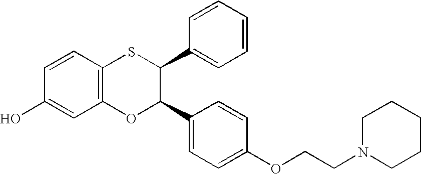

Step A

The dihydrobenzoxathiin generated from Example 55 was desilylated using the procedure described in Example 71 (Step C). The desired product was obtained as a white solid. ¹H NM (400 MHz, CDCl₃) δ (ppm: 7.48-7.32 (m, 5H), 7.2-7.1 (m, 4H), 6.94-6.84 (two d, 4H), 6.7 (m, 4H), 5.56 (d, J=2.1 Hz, 1H), 5.04 (br q, 2H), 4.74 (s, 1H), 4.37 (d, J=2.1 Hz, 1H).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained. ¹H NMR (400 MHz, CDCl₃) δ (ppm): 7.5-7.32 (m, 5H), 7.2-7.04 (m, 4H), 6.94-6.86 (m, 4H), 6.76-6.66 (m, 4H), 5.54 (br s, 1H), 5.04 (br s, 2H), 4.38 (br s, 1H), 4.06 (t, 2H), 2.76 (t, 2H), 2.5 (br s, 4H), 1.6 (m, 4H), 1.42 (m, 2H).

Step C

The adduct generated in Step B was debenzylated using the procedure described in Example 71 (Step B) to afford the desired product. ¹H NMR (400 MHz, CD₃OD) δ (ppm): 7.2-7.14 (m, 3H), 6.94 (m, 3H), 6.9 (d, 2H), 6.74 (d, 2H), 6.48 (dd, 1H), 6.45 (d, 1H), 5.53 (d, J=2.3 Hz, 1H), 4.46 (d, 1H), 4.06 (t, 2H), 2.78 (t, 2H), 2.58 (br s, 4H), 1.62 (m, 4H), 1.5 (m, 2H); MS m/z 449.2 (M⁺+1).

### EXAMPLE 82

### Preparation of

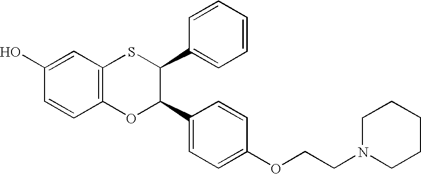

Step A

The dihydrobenzoxathiin generated from Example 56 was desilylated using the procedure described in Example 71 (Step C). The desired product was obtained as a white solid. ¹H NMR (400 MHz, CDCl₃) δ (ppm): 7.5-7.3 (m, 5H), 7.2-7.1 (m, 3H), 6.96 (m, 2H), 6.92 (d, 1H), 6.88 (d, 2H), 6.84 (d, 1H), 6.74 (dd, 1H), 6.66 (d, 2H), 5.48 (d, J=2.1 Hz, 1H), 5.04 (s, 2H), 4.37 (d, J=2.1 Hz, 1H); MS m/z 428.2 (M⁺+1).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained.

Step C

The adduct generated in Step B was debenzylated using the procedure described in Example 71 (Step B) to afford the desired product. ¹H NMR (400 MHz, CD₃OD) δ (ppm): 7.14-7.02 (m, 3H), 6.92 (m, 4H), 6.8 (d, 1H), 6.74 (d, 2H), 6.58 (d, 1H), 6.51 (dd, 1H), 5.42 (br s, 1H), 4.45 (br s, 1H), 4.06 (t, 2H), 2.78 (t, 2H), 2.55 (br s, 4H), 1.6 (m, 4H), 1.5 (m, 2H); MS m/z 449.2 (M⁺+1).

### EXAMPLE 83

### Preparation of

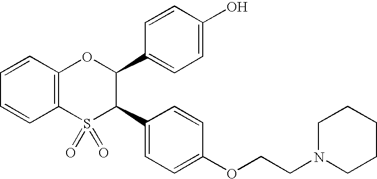

Step A

To a well stirred solution of the dihydrobenzoxathiin (30 mg, 0.061 mmole) prepared from Example 74 (Step A) was added 5equivalents of meta-chloroperbenzoic acid (m-CPBA) in methylene chloride at 0° C. The ice bath was removed and the reaction mixture was stirred at room temperature for three hours. The reaction mixture was quenched with a saturated solution of NaHSO₃ and stirred for additional 30 minutes. The aqueous layer was extracted with EtOAc and the organic layer was washed with brine, dried with MgSO₄, and evaporated to give a residue which was used for next step without further purification. ¹H NMR (400 MHz, CD₃OD) δ (ppm): 7.82 (dd, 1H), 7.67 (dt, 1H), 7.28 (m, 2H), 7.2 (d, 2H), 7.03 (d, 2H), 6.92 (d, 2H), 6.82 (d, 2H), 6.32 (d, 1H), 5.12 (s, 2H), 4.84 (d, 1H), 4.2 (br t, 2H), 3.40 (s, 3H), 3.2 (m, 2H), 3.0 (m, 4H), 1.75 (m, 4H), 1.6 (m, 2H).

Step B

The MOM protecting group was removed following the procedure outlined in Example 74 (Step B). The desired product was isolated after purification by silica gel chromatography using 5% MeOH/CH₂Cl₂ as the eluant. ¹H NMR (400 MHz, CD₃OD) δ (ppm): 7.82 (dd, 1H), 7.64 (dt, 1H), 7.26 (m, 2H), 7.04 (d, 2H), 6.06 (d, 2H), 6.76 (d, 2H), 6.65 (d, 2H), 6.24 (d, J=1.9 Hz, 1H), 4.71 (d, 1H), 4.1 (t, 2H), 2.72 (t, 2H), 2.5 (br s, 4H), 1.6 (m, 4H), 1.45 (m, 2H); MS m/z 481.1 (M⁺+1).

### EXAMPLE 84

### Preparation of

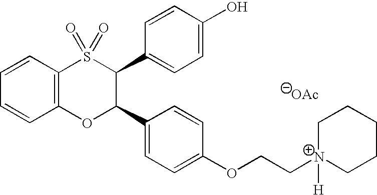

Step A

To a well stirred solution of the dihydrobenzoxathiin (60 mg) prepared from Example 73 (Step A) was added 5 equivalents of m-CPBA in CH₂Cl₂ at 0° C. The ice bath was removed and the reaction mixture was stirred at room temperature for 3 hours. The reaction mixture was quenched with a saturated solution of NaHSO₃ and saturated NaHCO₃, and stirred for additional 30 minutes. The aqueous layer was extracted with EtOAc and the combined organic layer was washed with brine and dried with MgSO₄. The solvent was removed by evaporation to give an oily residue, which was purified by silica gel chromatography with 3% MeOH(CH₂Cl₂ as the eluant to give the pure product. ¹H NMR (400 MHz, CD₃OD) δ (ppm): 7.85 (dd, 1H), 7.66 (m, 1H), 7.28 (m, 2H), 7.12 (d, 2H), 6.86 (d, 2H), 6.8 (d, 2H), 6.7 (d, 2H), 6.22 (d, J=2.1 Hz, 1H), 4.72 (d, J=2.3 Hz, 1H), 4.08 (m, 2H), 2.8 (t, 2H), 2.6 (br s, 4H), 1.6 (m, 4H), 1.5 (m, 2H), 1.22 (m, 3H), 1.1 (d, 18H); MS m/z 637 (M⁺+23).

Step B

The silyl protecting group was removed following the procedure outlined in Example 71 (Step C). The desired product was isolated after purification by silica gel chromatography using 5% MeOH(CH₂Cl₂ as the eluant. ¹H NMR (400 MHz, CD₃OD) δ (ppm): 7.81 (dd, 1H), 7.64 (m, 1H), 7.35 (m, 2H), 7.2 (d, 2H), 6.82 (two d, 4H), 6.6 (d, 2H), 6.28 (d, J=2.2 Hz, 1H), 4.69 (d, J=2.2 Hz, 1H), 4.2 (t, 2H), 3.08 (t, 2H), 2.85 (br s, 4H), 1.7 (m, 4H), 1.55 (m, 2H).

### EXAMPLE 85

### Preparation of

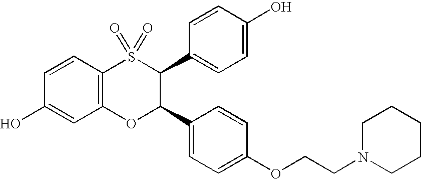

Step A

Utilizing the procedure from Example 83 (Step A), the dihydrobenzoxathiin (20 mg, 0.028 mmole) obtained from Example 71 (Step A), was oxidized by m-CPBA at room temperature. The crude material was used for next step without further purification. ¹H NMR (400 MHz, CDCl₃) δ (ppm): 7.84 (d, 1H), 7.7-7.4 (m, 5H), 7.02 (d, 2H), 6.88 (dd, 1H), 6.82 (d, 2H), 6.76 (two d, 4H), 6.72 (d, 1H), 6.22 (d, J=2.2 Hz, 1H), 5.18 (q, 2H), 4.28 (d, J=2.1 Hz, 1H), 4.09 (t, 2H), 2.8 (t, 2H), 2.55 (brs, 4H), 1.63 (m, 4H), 1.48 (m, 2H), 1.22 (m, 3H), 1.1 (d, 18H).

Step B

The product from Step A was deblocked using the standard procedure described in Example 71 (Step B) to afford the debenzylated product, which was used without further purification.

Step C

The silyl protecting group was removed following the procedure outlined in Example 71 (Step C). The final product was isolated after purification by silica gel chromatography using 5% MeOH/CH₂Cl₂ as the eluant. ¹H NMR (400 MHz, CD₃OD) δ (ppm): 7.62 (d, 1H), 7.14 (d, 2H), 6.84 (two d, 4H), 6.68 (dd, 1H), 6.6 (d, 2H), 6.55 (d, 1H), 6.22 (d, 1H), 4.55 (d, J=2.1 Hz, 1H), 4.1 (t, 2H), 2.8 (t, 2H), 2.6 (br s, 4H), 1.64 (M, 4H), 1.5 (M, 2H); MS m/z 496.1 (M⁺+1).

### EXAMPLE 86

### Preparation of

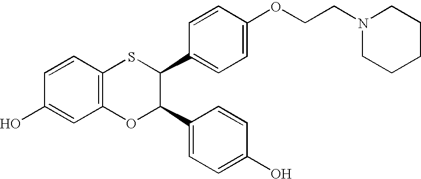

Step A

To a solution of dihydrobenzoxathiin (100 mg, 0.167 mmole) generated from Example 48 in CH₂Cl₂ was added triethylamine (0.07 mL), a catalytic amount of N,N-dimethylaminopyridine (DMAP) and acetic anhydride (0.034 mL, 2 eq) at room temperature. The resultant mixture was stirred for 30 minutes and then poured into saturated NaHCO₃. The aqueous layer was extracted with CH₂Cl₂ and then dried over anhydrous Na₂SO₄. The solvent was evaporated to give an oil, which was subjected to silica gel chromatography with 10% EtOAc/hexane as eluant to give the product. ¹H NMR (400 MHz, CDCl₃) δ (ppm): 7.48-7.34 (m, 5H), 7.08 (d, 1H), 6.99 (d, 2H), 6.94 (d, 2H), 6.76 (d, 2H), 6.72-6.67 (m, 4H), 5.56 (d, 1H), 5.06 (br q, 2H), 4.34 (d, 1H), 2.3 (d, 3H), 1.22 (m, 3H), 1.1 (d, 18 H).

Step B

The silyl protecting group was removed following the procedure outlined in Example 71 (Step C). The desired product was isolated after purification by silica gel chromatography using 5% MeOH/CH₂Cl₂ as the eluant. ¹H NMR (400 MHz, CDCl₃) δ (ppm): 7.48-7.34 (m, 5H), 7.09 (d, 1H), 7.04 (d, 2H), 6.98 (d, 2H), 6.78 (d, 2H), 6.7 (m, 2H), 6.59 (d, 2H), 5.56 (d, 1H), 5.06 (br q, 2H), 4.74 (s, 1H), 4.36 (d, 1H), 2.2 (s, 3H).

Step C

The desilylated product (80 mg, 0.165 mmole) obtained from Step B was coupled with 1-piperidineethanol using the procedure described in Example 71 (Step A). After purification by silica gel chromatography with 3% MeOH/CH₂Cl₂, the desired adduct was obtained. ¹H NMR (400 MHz, CDCl₃) δ (ppm): 7.48-7.34 (m, 5H), 7.08 (d, 1H), 7.04 (d, 2H), 6.98 (d, 2H), 6.82 (d, 2H), 6.7 (dd, 1H), 6.68 (d, 1H), 6.68 (d, 2H), 5.58 (d, J=2.2 Hz, 1H), 5.05 (br q, 2H), 4.36 (d, J=2.2 Hz, 1H), 4.05 (t, 2H), 2.68 (t, 2m), 2.5 (br s, 4H), 2.25 (s, 3H), 1.6 (m, 4H), 1.45 (m, 2H); MS m/z 597.3 (M⁺+1.

Step D

To a solution of 10 mg (0.017 mmole) of the adduct, generated from Step, in anhydrous THF was added four equivalents of a 1.0 M Super hydride solution in THF. The resulting mixture was stirred for 2 hours at 0° C. and then allowed to room temperature (30 minutes). The reaction mixture was hydrolyzed with H₂O/NaHCO₃. The aqueous layer was extracted with EtOAc, the organic layer separated, dried, and evaporated to give an oil, which was used for next step without further purification.

Step E

The crude product from Step D was deblocked using the standard procedure described in Example 71 (Step B) to afford the final product, after purification by silica gel chromatography using 5% MeOH/CH₂Cl₂ as the eluant. ¹H NMR (400 MHz, CD₃OD) δ (ppm): 6.92 (d, 1H), 6.83 (d, 2H), 6.82 (d, 2H), 6.65 (d, 2H), 6.58 (d, 2H), 6.46 (dd, 1H), 6.42 (d, 1H), 5.44 (d, J=2.1 Hz, 1H), 4.38 (d, 1H, J=2.3 Hz, 1H), 4.04 (t, 2H), 2.78 (t, 2H), 2.6 (br s, 4H), 1.6 (m, 4H), 1.5 (m, 2H); MS m/z 465 (M⁺+1).

### EXAMPLE 87

### Preparation of

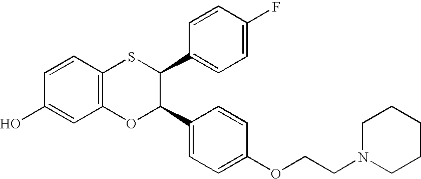

Step A

The desilylated product obtained from Example 57 was coupled with 1-piridineethanol using the procedure described in Example 71 (Step A). After purification by silica gel chromatography with 3% MeOH/CH₂Cl₂, the desired adduct was obtained.

Step B

The adduct generated in Step A was debenzylated using the procedure described in Example 71 (Step B) to afford the desired product. ¹H NMR (400 MHz, CD₃OD) δ (ppm): 6.98-6.76 (m, 9H), 6.5 (dd, 1H), 6.46 (d, 1H), 5.52 (d, J=2.3 Hz, 1H), 4.5 (d, 1H), 4.05 (t, 2H), 2.80 (t, 2H), 2.62 (br s, 4H), 1.62 (m, 4H), 1.5 (m, 2H); MS m/z 466.2 (M⁺).

### EXAMPLE 88

### Chiral Separation of

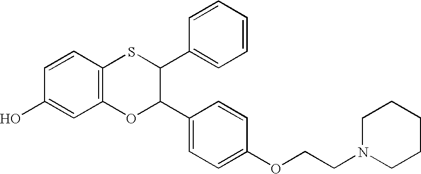

The racemic dihydrobenzoxathiin obtained from Example 81 (Step C) was resolved via chiral chromatography on a Chiralpak AD column, using 20% EtOH in hexane as the eluant. The fast moving isomer: [α]D=+33.43°(c=1.205, MeOH). The slow moving isomer: [α]D=−34.2°(c=1.09, MeOH).

### EXAMPLE 89

### Chiral Separation of

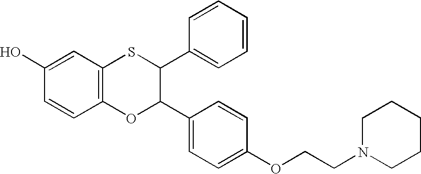

The racemic dihydrobenzoxathiin obtained from Example 82 (Step C) was resolved via chiral chromatography on a Chiralpak AD column, using 20% EtOH in hexane as the eluant. The fast moving isomer: [α]D=+32.4°(c=1.36, MeOH). The slow moving isomer: [α]D=−31.3°(c=1.37, MeOH).

### EXAMPLE 90

### Preparation of

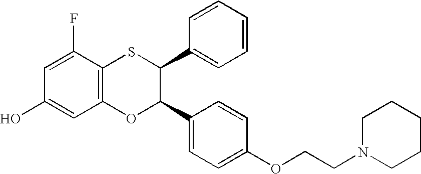

The dihydrobenzoxathiin generated from Example 58 was desilylated using the procedure described in Example 71 (Step C). The desired product was obtained as a white solid. ¹H NMR (500 MHz, CDCl₃) δ (ppm): 7.5-7.3 (m, 5H), 7.2-7.1 (m, 3H), 6.85 (2d, 4H), 6.68 (d, 2H), 6.55 (d, 2H), 5.55 (d, 1H), 5.04 (s, 2H), 4.40(d, 1H).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained.

Step C

A mixture of the adduct (80 mg, 0.144 mmole), generated in Step B, 20 mg of palladium black and 5 drops of AcOH in 4 mL of ethanol, was stirred under a balloon of hydrogen gas and monitored by TLC. After 18 hours, the reaction mixture was filtered through a pad of Celite to remove the catalyst, and the filtrate was neutralized by the addition of saturated, aqueous NaHCO₃ solution and extracted by EtOAc. The organic layer was separated, dried over MgSO₄ and concentrated in vacuo to give the desired product. ¹H NMR (500 MHz, CD₃OD) δ (ppm): 7.20-7.02 (m, 3H), 6.92 (m, 4H), 6.78 (d, 2H), 6.30 (d, 2H), 5.55 (d, J=2.1 Hz, 1H), 4.50(d, J=2.3 Hz, 1H), 4.06 (t, 2H), 2.78 (t, 2H), 2.55 (br s, 4H), 1.6 (m, 4H), 1.5 (m, 2H); MS m/z 467 (M⁺+1).

### EXAMPLE 91

### Preparation of

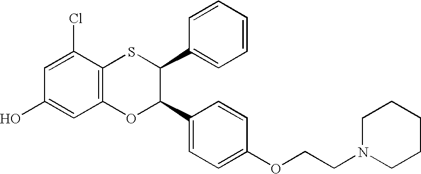

Step A

The dihydrobenzoxathiin generated from Example 59 was desilylated using the procedure described in Example 71 (Step C). The desired product was obtained as a white solid. ¹H NMR (500 MHz, CDCl₃) δ (ppm): 7.5-7.3 (m, 5H), 7.2-7.1 (m, 3H), 6.95 (d, 2H), 6.90 (d, 1H), 6.85 (d, 2H), 6.70 (d, 2H), 6.65 (d, 1H), 5.50 (d, 1H), 5.04 (s, 2H), 4.42 (d, 1H).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained.

Step C

The adduct, generated in Step B, was debenzylated using the procedure described in Example 71 (Step B) to afford the desired product. ¹H NMR (500 MHz, CD₃OD) δ (ppm): 7.14-7.02 (m, 3H), 6.92 (d, 2H), 6.85 (d, 2H), 6.74 (d, 2H), 6.58 (d, 1H), 6.41 (d, 1H), 5.52 (d, J=2.3 Hz, 1H), 4.55 (d, J=2.3 Hz, 1H), 4.06 (t, 2H), 2.78 (t, 2H), 2.55 (br s, 4H), 1.6 (m, 4H), 1.5 (m, 2H); MS m/z 483 (M⁺+1).

### EXAMPLE 92

### Preparation of

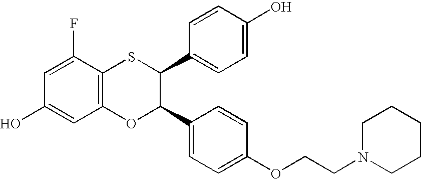

Step A

The dihydrobenzoxathiin, obtained from Example 60, was coupled with 1-piperidineethanol using the procedure described in Example 71 (Step A). After purification by silica gel chromatography with 3% MeOH/CH₂Cl₂ the desired adduct was obtained. ¹H NMR (500 MHz, CDCl₃) δ (ppm): 7.5-7.3 (m, 5H), 6.80 (d, 2H), 6.70 (2d, 4H), 6.60 (d, 2H), 6.40 (2d, 2H), 5.40 (s, 1H), 4.90 (d, 2H), 4.20 (s, 1H), 4.08 (t, 2H), 2.8 (t, 2H), 2.5 (br s, 4H), 1.62 (m, 4H), 1.5 (m, 2H), 1.22 (m, 3H), 1.1 (d, 18H).

Step B

The adduct, generated in Step A, was debenzylated using the procedure described in Example 71 (Step B).

Step C

The debenzylated product from Step B was desilylated using the procedure described in Example 71 (Step C). The desired product was obtained as a white solid. ¹H NMR (500 MHz, CD₃OD) δ (ppm): 6.93 (d, 3H), 6.78 (d, 2H), 6.69 (d, 2H), 6.50 (d, 2H), 6.28 (m, 1H), 5.46 (d, J=1.8 Hz, 1H), 4.39 (d, J=2.2 Hz, 1H), 4.05 (t, 2H), 2.8 (t, 2H), 2.6 (br s, 4H), 1.64 (m, 4H), 1.5 (m, 2H), MS m/z 482.2 (M⁺+1).

### EXAMPLE 93

### Preparation of

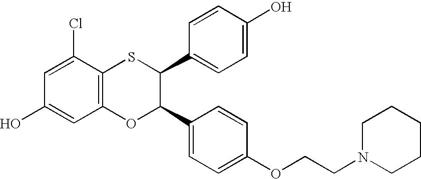

Step A

The dihydrobenzoxathiin, obtained from Example 61, was coupled with 1-piperidineethanol using the procedure described in Example 71 (Step A). After purification by silica gel chromatography with 3% MeOH/CH₂Cl₂ the desired adduct was obtained. ¹H NMR (500 MHz, CDCl₃) δ (ppm): 7.5-7.3 (m, 5H), 6.85 (m, 3H), 6.70 (d, 4H), 6.63 (d, 2H), 6.60 (d, 1H), 5.42 (s, 1H), 5.02 (d, 2H), 4.40 (s, 1H), 4.08 (t, 2H), 2.8 (t, 2H), 2.5 (br s, 4H), 1.62 (m, 4H), 1.5 (m, 2H), 1.22 (m, 3H), 1.1 (d, 18H).

Step B

The adduct, generated in Step A, was debenzylated using the procedure described in Example 71 (Step B) to afford the desired product. ¹H NMR (500 MHz, CD₃OD) δ (ppm): 6.82 (d, 2H), 6.78 (d, H), 6.70 (2d, 4H), 6.62 (d, 2H), 6.58 (d, 1H), 5.40 (d, 1H), 4.30 (d, 1H), 4.06 (t, 2H), 2.78 (t, 2H), 2.55 (br s, 4H), 1.6 (m, 4H), 1.5 (m, 2H); MS m/z 655 (M⁺+1).

Step C

The debenzylated product from Step B was desilylated using the procedure described in Example 71 (Step C). The desired product was obtained as a white solid. ¹H NMR (500 MHz, CD₃OD) δ (ppm): 6.92 (d, 2H), 6.75 (d, 2H), 6;68(d, 2H), 6.60 (d, 1H), 6.50 (d, 2H), 6.42(d, 1H), 5.42 (d, J=2.2 Hz, 1H), 4.42 (d, J=2.3 Hz, 1H), 4.07 (t, 2H), 2.78 (t, 2H), 2.55 (brs, 4H), 1.62 (m, 4H), 1.48 (m, 2H); MS m/z 499 (M⁺+1).

### EXAMPLE 94

### Preparation of

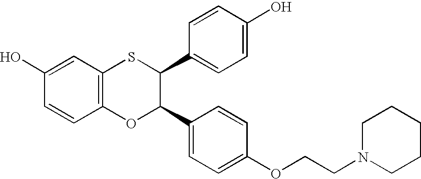

Step A

The dihydrobenzoxathiin, obtained from Example 62, was coupled with 1-piperidineethanol using the procedure described in Example 71 (Step A). After purification by silica gel chromatography with 3% MeOH/CH₂Cl₂ the desired adduct was obtained.

Step B

The adduct, generated in Step A, was debenzylated using the procedure described in Example 71 (Step B).

Step C

The debenzylated product from Step B was desilylated using the procedure described in Example 71 (Step C). The desired product was obtained as a white solid after purification by silica gel chromatography with 5% MeOH/CH₂Cl₂ as eluant. ¹H NMR (500 MHz, acetone-d₆) δ (ppm): 7.04 (d, 2H), 6.90 (dd, 3H), 6.72 (d, 2H), 6.64 (d, 1H), 6.59 (d, 2H), 6.57(dd, 1H), 5.44 (d, J=2.3 Hz, 1H), 4.52 (d, J=2.1 Hz, 1H), 4.08 (t, 2H), 2.8 (t, 2H), 2.62 (br s, 4H), 2.6 (q, 2H), 1.6 (m, 4H), 1.45 (m, 2H), 1.2 (t, 2H); MS m/z 465 (M⁺+1).

### EXAMPLE 95

### Preparation of

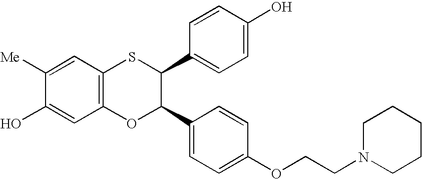

Step A

The dihydrobenzoxathiin, obtained from Example 63, was coupled with 1-piperidineethanol using the procedure described in Example 71 (Step A). After purification by silica gel chromatography with 3% MeOH/CH₂Cl₂ the desired adduct was obtained.

Step B

The adduct, generated in Step A, was debenzylated using the procedure described in Example 71 (Step B).

Step C:

The debenzylated product from Step B was desilylated using the procedure described in Example 71 (Step C). The desired product was obtained as a white solid after purification by silica gel chromatography with 5% MeOH(CH₂Cl₂ as eluant. ¹H NMR (500 MHz, acetone-d₆) δ (ppm): 7.00 (d, 2H), 6.85 (s, 1H), 6.80 (d, 2H), 6.78 (d, 2H), 6.59 (d, 2H), 6.52 (s, 1H), 5.49 (d, J=2.3 Hz, 1H), 4.65(d, J=2.2 Hz, 1H), 4.08 (t, 2H), 2.8 (t, 2H), 2.62 (br s, 4H), 2.6 (q, 2H), 1.6 (m, 4H), 1.45 (m, 2H), 1.2 (t, 2H); MS m/z 479 (M⁺+1).

### EXAMPLE 96

### Preparation of

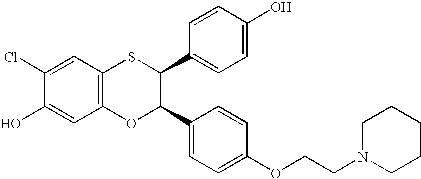

Step A

The dihydrobenzoxathiin, obtained from Example 64, was coupled with 1-piperidineethanol using the procedure described in Example 71 (Step A). After purification by silica gel chromatography with 3% MeOH/CH₂Cl₂ the desired adduct was obtained. ¹H NMR (500 MHz, CDCl₃) δ (ppm): 7.5-7.3 (m, 5H), 7.20 (s, 1H), 6.85 (d, 2H), 6.70 (2d, 4H), 6.63 (d, 2H), 6.60 (s, 1H), 5.42 (s, 1H), 5.02 (q, 2H), 4.30 (s, 1H), 4.08 (t, 2H), 2.8 (t, 2H), 2.5 (br s, 4H), 1.62 (m, 4H), 1.5 (m, 2H), 1.22 (m, 3H), 1.1 (d, 18H).

Step B

The adduct, generated in Step A, was debenzylated using the procedure described in Example 71 (Step B) to afford the desired product. ¹H NMR (500 MHz, acetone-d₆) δ (ppm): 7.10 (s, 1H), 6.98 (d, 2H), 6.82 (d, 2H), 6.78 (d, 2H), 6.70 (d, 2H), 6.68 (s, 1H), 5.50 (d, 1H), 4.50 (d, 1H), 4.06 (t, 2H), 2.78 (t, 2H), 2.55 (br s, 4H), 1.6 (m, 4H), 1.5 (m, 2H).

Step C

The debenzylated product from Step B was desilylated using the procedure described in Example 71 (Step C). The desired product was obtained as a white solid. ¹H NMR (500 MHz, acetone-d₆) δ (ppm): 7.12 (s, 1H), 7.02 (d, 2H), 6.80 (dd, 4H), 6.69 (s, 1H), 6.60 (d, 2H), 6.42 (d, 1H), 5.55 (d, J=2.3 Hz, 1H), 4.54 (d, J=2.1 Hz, 1H), 4.07 (t, 2H), 2.78 (t, 2H), 2.55 (brs, 4H), 1.62 (m, 4H), 1.48 (m, 2H); MS m/z 499 (M⁺+1).

### EXAMPLE 97

### Preparation of

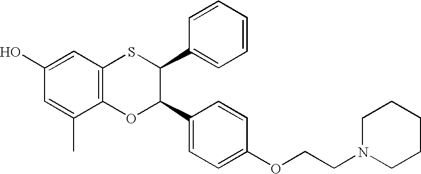

Step A

The dihydrobenzoxathiin generated from Example 65 was desilylated using the procedure described in Example 71 (Step C). The desired product was obtained as a white solid. ¹H NMR (500 MHz, CDCl₃) δ (ppm): 7.5-7.3 (m, 5H), 7.2-7.1 (m, 5H), 6.95 (m, 3H), 6.64-6.70 (m, 2H), 5.46 (d, J=1.8 Hz, 1H), 5.04 (s, 2H), 4.42 (d, J=2.0 Hz, 1H).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained.

Step C

The adduct, generated in Step B, was debenzylated using the procedure described in Example 71 (Step B) to afford the desired product. ¹H NMR (500 MHz, CD₃OD) δ (ppm: 7.00-7.12 (m, 6H), 6.90 (d, 2H), 6.75 (d, 2H), 6.42 (s, 1H), 5.42 (d, J=2.1 Hz, 1H), 4.48 (d, J=2.3 Hz, 1H), 4.06 (t, 2H), 2.78 (t, 2H), 2.55 (br s, 4H), 1.6 (m, 4H), 1.5 (m, 2H); MS m/z 463 (M⁺+1).

### EXAMPLE 98

### Preparation of

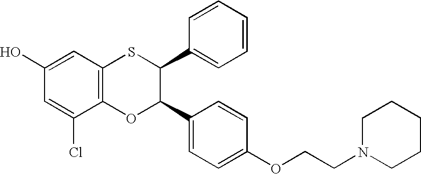

Step A

The dihydrobenzoxathiin generated from Example 66 was desilylated using the procedure described in Example 71 (Step C). The desired product was obtained as a white solid. ¹H NMR (500 MHz, CDCl₃) δ (ppm): 7.5-7.3 (m, 5H), 7.2-7.1 (m, 3H), 6.95 (d, 2H), 6.92 (d, 2H), 6.90 (d, 1H), 6.78 (d, 1H), 6.70 (d, 2H), 5.52 (d, J=2.1 Hz, 1H), 5.04 (s, 2H), 4.46 (d, J=2.2 Hz, 1H).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained.

Step C

The adduct, generated in Step B, was debenzylated using the procedure described in Example 71 (Step B) to afford the desired product. ¹H NMR (500 MHz, CD₃OD) δ (ppm): 7.05-7.15 (m, 5H), 6.90 (d, 2H), 6.79 (d, 2H), 6.65 (d, 1H), 6.55 (d, 1H), 5.50 (d, J=2.1 Hz, 1H), 4.62 (d, J=2.3 Hz, 1H), 4.10 (t, 2H), 2.80 (t, 2H), 2.60 (br s, 4H), 1.6 (m, 4H), 1.5 (m, 2H); MS m/z 483 (M⁺+1).

### EXAMPLE 99

### Preparation of

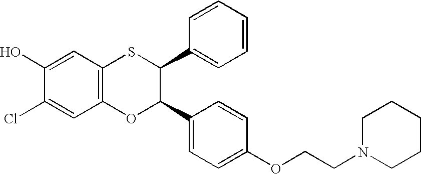

Step A

The dihydrobenzoxathiin generated from Example 67 was desilylated using the procedure described in Example 71 (Step C). The desired product was obtained as a white solid. ¹H NMR (500 MHz, CDCl₃) δ (ppm): 7.5-7.3 (m, 5H), 7.2-7.1 (m, 3H), 7.08 (s, 1H), 6.95 (d, 2H), 6.86 (m, 3H), 6.70 (d, 2H), 5.42 (d, J=2.1 Hz, 1H), 5.14 (s, 2H), 4.40 (d, J=2.0 Hz, 1H).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained.

Step C

The adduct, generated in Step B, was debenzylated using the procedure described in Example 71 (Step B) to afford the desired product. ¹H NMR (500 MHz, CD₃OD) δ (ppm): 7.05-7.15 (m, 3H), 6.95 (m, 3H), 6.90 (d, 2H), 6.75 (d, 2H), 6.72 (s, 1H), 5.45 (d, J=2.0 Hz, 1H), 4.52 (d, J=2.3 Hz, 1H), 4.10 (t, 2H), 2.80 (t, 2H), 2.60 (br s, 4H), 1.6 (m, 4H), 1.5 (m, 2H); MS m/z 483 (M⁺+1).

### EXAMPLE 100

### Preparation of

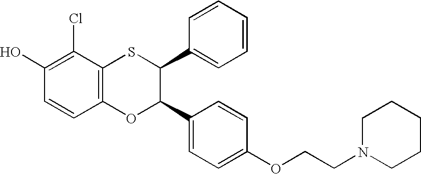

Step A

The dihydrobenzoxathiin generated from Example 68 was desilylated using the procedure described in Example 71 (Step C). The desired product was obtained as a white solid. ¹H NMR (500 MHz, CDCl₃) δ (ppm): 7.5-7.3 (m, 5H), 7.2-7.1 (m, 3H), 6.92-6.80 (m, 5H), 6.78 (d, 2H), 6.70 (d, 2H), 5.40 (d, J=2.1 Hz, 1), 5.20 (s, 2H), 4.46 (d, J=2.0 Hz, 1H).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained.

Step C

The adduct, generated in Step B, was debenzylated using the procedure described in 71 (Step B) to afford the desired product. ¹H NMR (500 MHz, CD₃OD) δ (ppm): 7.05-7.15 (m, 3H), 6.95 (d, 2H), 6.90 (d, 2H), 6.80 (d, 1H), 6.75 (d, 2H), 6.70 (d, 1H), 5.38 (d, J=1.8 Hz, 1H), 4.56 (d, J=2.1 Hz, 1H), 4.06 (t, 2H), 2.78 (t, 2H), 2.60 (br s, 4H), 1.6 (m, 4H), 1.5 (m, 2H); MS m/z 483 (M⁺+1).

### EXAMPLE 101

### Chiral Separation of

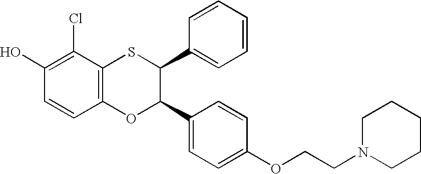

The racemic dihydrobenzoxathiin obtained from Example 100 (Step C) was resolved via chiral chromatography on a Chiralpak AD column, using 20% EtOH in hexane as the eluant. The fast moving isomer: [α]D=+26.09°(c=1.025, MeOH). The slow moving isomer: [α]D=−25.44°(c=0.95, MeOH).

### EXAMPLE 102

### Preparation of

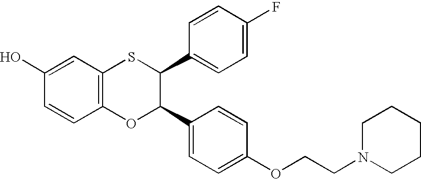

Step A

The dihydrobenzoxathiin generated from Example 69 was desilylated using the procedure described in Example 71 (Step C). The desired product was obtained as a white solid. ¹H NMR (500 MHz, CDCl₃) δ (ppm): 7.5-7.3 (m, 5H), 6.95 (d, 2H), 6.90(m, 3H), 6.85 (m, 3H), 6.74 (dd, 1H), 6.70 (d, 2H), 5.45 (d, J=1.9 Hz, 1H), 5.05 (s, 2H), 4.35 (d, J=2.1 Hz, 1H).

Step B

The desilylated product obtained from Step A was coupled with 1-piperidineethanol using the procedure described in Example 71 (Step A). After purification by silica gel chromatography with 3% MeOH/CH₂Cl₂, the desired adduct was obtained, which was used without further purification.

Step C

The adduct, generated in Step B, was debenzylated using the procedure described in Example 71 (Step B) to afford the desired product. ¹H NMR (500 MHz, CD₃OD) δ (ppm): 6.98 (d, 2H), 6.94 (m, 2H), 6.80 (m, 5H), 6.60 (d, 1H), 6.75 (dd, 1H), 5.40 (d, J=1.8 Hz, 1H), 4.50 (d, J=2.1 Hz, 1H), 4.08 (t, 2H), 2.78 (t, 2H), 2.60 (br s, 4H), 1.6 (m, 4H), 1.5 (m, 2H); MS m/z 466 (M⁺+1).

### EXAMPLE 103

### Chiral Preparation of

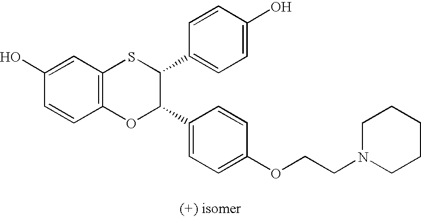

Step A

The fast moving (+)-dihydrobenzoxathiin obtained from Example 70 was coupled with 1-piperidineethanol using the procedure described in Example 71 (Step A). After purification by silica gel chromatography with 3% MeOH/CH₂Cl₂, the desired adduct was obtained.

Step B

The adduct, generated in Step A, was debenzylated using the procedure described in Example 71 (Step B).

Step C

The debenzylated product from Step B was desilylated using the procedure described in Example 71 (Step C). The desired product was obtained as a white solid after purification by silica gel chromatography with 5% MeOH/CH₂Cl₂ as eluant. ¹H NMR (500 MHz, acetone-d₆) δ (ppm): 6.90 (d, 2H), 6.78 (d, 1H), 6.72 (d, 2H), 6.70 (d, 2H), 6.60 (d, 1H), 6.50 (d, 1H), 6.48 (d, 2H), 5.38 (d, J=2.0 Hz, 1H), 4.38 (d, J=2.3 Hz, 1H), 4.08 (t, 2H), 2.8 (t, 2H), 2.62 (br s, 4H), 2.6 (q, 2H), 1.6 (m, 4H), 1.45 (m, 2H), 1.2 (t, 2H); MS m/z 465 (M⁺+1); [α:]_{D}=+27.68°(c=0.49, MeOH).

### EXAMPLE 104

### Chiral Preparation of

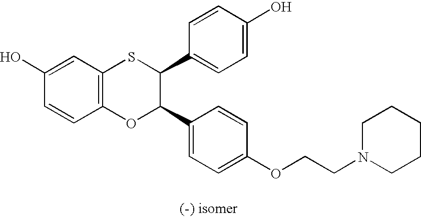

Step A

The slow moving (−)-dihydrobenzoxathiin obtained from Example 70 was coupled with 1-piperidineethanol using the procedure described in Example 71 (Step A). After purification by silica gel chromatography with 3% MeOH/CH₂Cl₂, the desired adduct was obtained.

Step B

The adduct, generated in Step A, was debenzylated using the procedure described in Example 71 (Step B).

Step C

The debenzylated product from Step B was desilylated using the procedure described in Example 71 (Step C). The desired product was obtained as a white solid after purification by silica gel chromatography with 5% MeOH/CH₂Cl₂ as eluant. ¹H NMR (500 MHz, acetone-d₆) δ (ppm): 6.90 (d, 2H), 6.78 (d, 1H), 6.72 (d, 2H), 6.70 (d, 2H), 6.60 (d, 1H), 6.50 (d, 1H), 6.48 (d, 2H), 5.38 (d, J=2.0 Hz, 1H), 4.38 (d, J=2.3 Hz, 1H), 4.08 (t, 2H), 2.8 (t, 2H), 2.62 (br s, 4H), 2.6 (q, 2H), 1.6 (m, 4H), 1.45 (m, 2H), 1.2 (t, 2H); MS m/z 465 (M⁺+1); [α]_{D}=−26.33°(c=0.515, MeOH).

### EXAMPLE 105

### General Preparation of

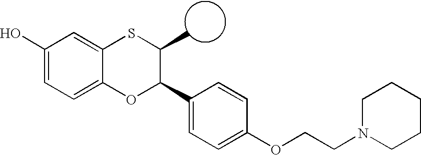

Step A: Reductive Cyclization

To a stirred solution of 102.2 mg (0.17 mmole) of the cyclopentyl-thio-ketone generated in Example 41 in 1 mL of dichloromethane at −23° C. under an N₂ atmosphere was added 68 μL (0.087 mmole) of neat trifluoroacetic acid(TFA). To the stirred reaction mixture at −23° C. was slowly added 41.4mL (0.259 mmole) of neat triethylsilane and the resulting mixture was stirred further for three hours. The reaction mixture was partitioned between ethyl acetate/saturated NaHCO₃/ice/brine, and the organic phase was separated, washed with brine, dried over anhydrous sodium sulfate, filtered, and evaporated. The residue was purified by silica gel chromatography using methylene chloride/hexanes (1:1) as eluant to provide the cis-cyclopentyl-dihydrobenzooxathiin derivative. ¹H 500 MHz NMR(CDCl₃) ppm(δ): 1.12 (d, 18H), 1.26-2.12 (m, 12H), 2.5 (m, 1H), 4.24 (d, 1H), 4.9 (m, 2H), 6.8-7.69 (m, 12H).

Starting with the cyclohexyl derivative prepared in Example 41 and utilizing the above procedure the corresponding cis-cyclohexyl-benzooxathiin was prepared after purification by silica gel chromatography using methylene chloride-hexanes (1:1). ¹H 500 MHz NMR(CDCl₃) ppm(δ): 1.14 (d, 18H), 1.11-1.9 (m, 14H), 3.2 (t, 1H), 5.03 (s, 2H), 5.44 (d, J=2.5 Hz, 1H), 6.66-7.47 (m, 12H).

Step B: Desilylation

To a stirred solution of 89.6mg (0.156 mmole) of the cis-cyclopentyl derivative prepared in Step A above in 1 mL of THF at 0° C. was added sequentially 13.3 μL (0.234 mmole) of acetic acid and then 171 μL (0.171 mmole) of a 1M solution of tetrabutylammonium fluoride in THF. The mixture was stirred at 0° C. for 0.5 hour and then partitioned between ethyl acetate/2N HCl/ice/brine, and the organic phase was separated, washed with brine, dried over anhydrous sodium sulfate, filtered, and evaporated. The residue was purified by silica gel chromatography using methylene chloride-ethyl acetate (50:1) as eluant to provide the phenolic derivative. ¹H 500 MHz NMR(CDCl₃) ppm(δ): 1.32-1.94 (m, 9H), 3.51 (dd, J=5.5, 2.5 Hz, 1H), 5.03 (s, 2H), 5.42 (d, J=2.3 Hz, 1H), 6.67-7.47 (m, 12H).

Starting with the cyclohexyl derivative prepared in the previous example and utilizing the above procedure the corresponding cis-cyclohexyl-benzooxathiin phenol was prepared. ¹H 500 MHz NMR(CDCl₃) ppm(δ): 1.11-1.93 (m, 11H), 3.23 (t, J=3 Hz, 1H), 5.03 (s, 2H), 5.44 (d, J=2.3 Hz, 1H), 6.66-7.47 (m, 12H).

Step C: Mitsunobu Reaction

To a stirred solution of a mixture of 56.3 mg (0.135 mmole) of the cis-cyclopentyl derivative prepared in Step B above, 53.6 μL (0.404 mmole) of 1-piperidineethanol, and 123.5 mg (0.47 mmole) of triphenylphosphine in 1 mL of anhydrous THF at 0° C. was added 87.4 μL (0.444 mmole) of neat diisopropylazodicarboxylate (DIAD). The ice-water bath was removed and the mixture was stirred further for six hours. The mixture was partitioned between ethyl acetate/2N HCl/ice/brine, and the organic phase was separated, washed with brine, dried over anhydrous sodium sulfate, filtered, and evaporated. The residue was purified by silica gel chromatography using ethyl acetate-methanol (9:1) as eluant to provide the adduct. ¹H 500 MHz NMR(CDCl₃) ppm(δ): 1.33-2.0 (m, 15H), 2.56 (m, 4H), 2.82 (t, J=6 Hz, 2H), 3.51 (dd, J=5.4, 2.4 Hz, 1H), 4.16 (t, J=6 Hz, 2H), 5.02 (s, 2H), 5.42 (d, J=2.3 Hz, 1H), 6.66-7.46 (m, 12H).

Starting with the cyclohexyl derivative prepared in the previous example and utilizing the above procedure the corresponding cis-cyclohexyl-benzooxathiin adduct was prepared. ¹H 500 MHz NMR(CDCl₃) ppm(δ): 1.11-1.93 (m, 17H), 2.6 (m, 4H), 2.87 (m, 2H), 3.2 (d, J=2.5 Hz, 1H), 4.2 (m, 2H), 5.02 (s, 2H), 5.44 (d, J=2.1 Hz, 1H), 6.65-7.46 (m, 12H).

Step D: Debenzylation:

A stirred mixture of 36.6 mg (0.0069 mmole) of the cis-cyclopentyl derivative prepared in Step C above, 14.7 mg (0.014 mmole) of palladium black, and 87.1 mg (0.138 mmole) of ammonium formate in 2 mL of ethanol-ethyl acetate-water (7:2:1) was heated at 80° C. for two hours. The mixture was filtered through celite, washed well with ethyl acetate and the filtrate was partitioned between ethyl acetate/saturated sodium bicarbonate/brine, and the organic phase was separated, washed with brine, dried over anhydrous sodium sulfate, filtered, and evaporated. The residue was purified by silica gel chromatography using ethyl acetate-methanol (9:1) as eluant to provide the final product. ¹H 500 MHz NMR(CDCl₃) ppm(δ): 1.33-2.0 (m, 15H), 2.6 (m, 4H), 2.88 (m, 2H), 3.48 (t, J=2.3 Hz, 1H), 4.18 (m, 2H), 5.38 (d, J=2.3 Hz, 1H), 6.5 (m, 1H), 6.63 (d, 2.9 Hz, 1H) 6.74 (d, J=8.7 Hz, 1H), 6.89 (d, J=8.7 Hz, 2H), and 7.34 (d, J=8.7 Hz, 2H).

Starting with the cyclohexyl derivative prepared in the previous example and utilizing the above procedure the corresponding cis-cyclohexyl-benzooxathiin adduct was prepared. ¹H 500 MHz NMR(CDCl₃) ppm(δ): 1.00-1.90 (m, 18H), 2.6 (m, 4H), 2.81 (t, 2H), 3.19 (t, J=3.0 Hz, 1H), 4.18 (m, 2H), 5.38 (d, J=2.3 Hz, 1H), 6.43 (m, 1H), 6.62 (d, J=3.0 Hz, 1H), 6.68 (d, J=8.7 Hz, 1H), 6.87 (d, J=8.7 Hz, 2 H), and 7.34 (d, J=8.7 Hz, 2H); MS m/z 454 (M⁺).

### EXAMPLE 106

### Preparation of

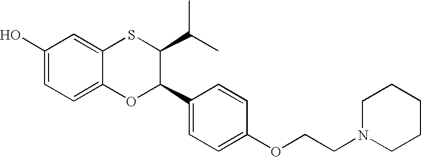

Step A: Reductive Cyclization

Starting with the isopropyl adduct (0.0208 g, 0.049 mmol) prepared in Example 42 and utilizing the procedure outlined in Example 105 (Step A), the crude product was isolated after stirring at −23° C. for 6 h 20 min. Purification by silica gel chromatography with 30% EtOAc/hexane as the eluant afforded the desired product as a yellow oil. ¹H 500 MHz NMR(CDCl₃) ppm(δ): 0.95 (d, 3H), 0.98 (d, 3H), 1.95 (m, 1H), 3.30 (t, J=3 Hz, 1H), 5.03 (s, 2H), 5.42 (d, J=2.6 Hz, 1H), 6.66-7.47 (m, 12H).

Step B: Mitsunobu Reaction

The dihydrobenzoxathiin prepared in Step A above was coupled with 1-piperidineethanol using the procedure described in Example 105 (Step C) with the exception that the reaction was allowed to slowly warm from 0° C. to ambient temperature over 3.5 h. Purification by silica gel chromatography with 10% MeOH/CH₂Cl₂ as the eluant afforded the desired product as a pale yellow oil. ¹H 500 MHz NMR(CDCl₃) ppm(δ): 0.95 (d, 3H), 0.98 (d, 3H), 1.50-1.68 (m, 6H), 1.95 (m, 1H), 2.60 (m, 4H), 2.86 (t, 2H), 3.30 (t, J=3 Hz, 1H), 4.20 (t, 2H), 5.03 (s, 2H), 5.42 (d, J=2.6 Hz, 1H), 6.66-7.49 (m, 12H).

Step C: Debenzylation

Starting with the compound prepared in Step B above, and utilizing the procedure outlined in Example 105 (Step D), the corresponding cis-isopropyl-benzoxathiin adduct was prepared after silica gel chromatography with 10% MeOH/CH₂Cl₂ as the eluant. ¹H 500 MHz NMR(CDCl₃) ppm(δ): 0.95 (d, 3H), 0.98 (d, 3H), 1.50-1.68 (m, 6H), 1.95 (m, 1H), 2.60 (m, 4H), 2.86 (t, 2H), 3.26 (t, J=3.0 Hz, 1H), 4.20 (t, 2H), 5.37 (d, J=2.5 Hz, 1H), 6.47 (dd, 1H), 6.65 (d, J=3 Hz, 1H), 6.72 (d, J=8.6 Hz, 2H), and 7.35 (d, J=8.7 Hz, 2H); MS m/z 414 (M⁺).

### EXAMPLE 107

### Preparation of

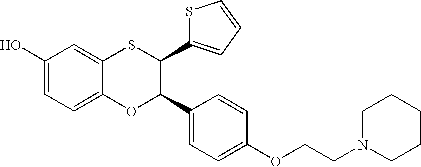

Step A: Reductive Cyclization

Starting with the 2-thiophene adduct (0.0208 g, 0.049 mmol) prepared in Example 43 and slightly modifying the procedure outlined in Example 105 (Step A), the crude product was isolated after stirring at 0° C. to ambient temperature for 1 h 40 min. Purification by silica gel chromatography with 30% EtOAc/hexane as the eluant afforded the desired product as a red oil. ¹H 500 MHz NMR(CDCl₃) ppm(δ): 1.11 (d, 18H), 1.24 (m, 3H), 4.67 (d, J=2.0 Hz, 1H), 5.50 (d, J=1.8 Hz, 1H), 6.60-7.12 (m, 10H).

Step B: Protection with MOM

To a solution of the didhydrobenzoxathiin (0.0629 g, 0.13 mmol) prepared in Step A above in distilled THF (1 mL) was added 60% NaH in mineral oil (0.0090 g, 0.19 mmol) at 0° C. under N₂. After the-gas evolution had ceased, MOMCl (0.013 mL, 0.16 mmol) was added dropwise to the reaction. After 30 min., another 1.3 equivalents of MOMCl was added to the reaction. Within 5 min., the reaction was complete by TLC. The resulting dark red solution was partitioned between EtOAc and ice/H₂O. The organic layer was washed with brine, dried over Na₂SO₄, and concentrated in vacuo. The desired product was used in the next reaction without purification. ¹H 500 MHz NMR(CDCl₃) ppm(δ): 1.11 (d, 18H), 1.24 (m, 3H), 3.52 (s, 3H), 4.67 (d, J=2.1 Hz, 1H), 5.14 (m, 2H), 5.50 (d, J=1.8 Hz, 1H), 6.60-7.12 (m, 10H).

Step C: Desilylation

The dihydrobenzoxathiin prepared in Step B above was. desilylated using the procedure described in Example 105 (Step B) to afford the desired product as a colorless oil after silica gel chromatography with 30% EtOAc/hexane as the eluant. ¹H 500 MHz NMR(CDCl₃) ppm(δ): 3.52 (s, 3H), 4.69 (d, J=1.8 Hz, 1H), 5.15 (m, 2H), 5.51 (d, J=1.8 Hz, 1H), 6.60-7.15 (m, 10H).

Step D: Mitsunobu Reaction

Following the procedure detailed in Example 105 (Step C) with the exception that the reaction was allowed to warm from 0° C. to ambient temperature over 4 h, the material prepared in the previous step was converted to the desired product after silica gel chromatography (one elution with 30% EtOAc/hexane followed by a second elution with 10% MeOH/CH₂Cl₂). ¹H 500 MHz NMR(CDCl₃) ppm(δ): 1.40-2.60 (m, 10H), 2.79 (t, 2H), 3.52 (s, 3H), 4.10 (t, 2H), 4.69 (d, J=1.8 Hz, 1H), 5.15 (m, 2H), 5.51 (d, J=1.8 Hz, 1H), 6.60-7.15 (m, 10H).

Step E: Deprotection of MOM

A mixture of the material (0.0401 g, 0.080 mmol) prepared in Step D above and 2 N HCl (0.20 mL, 0.40 mmol) in MeOH (1.0 mL) was heated to 60° C. under N₂ for 2.5 h. The reaction was partitioned between EtOAc and ice/sat. NaHCO₃. The organic layer was washed with brine, dried over Na₂SO₄, and concentrated in vacuo. The residue was triturated with Et₂O and desired product was obtained as a white solid. ¹H 500 MHz NMR(d₆-acetone+CD₃OD) ppm(δ): 1.50-3.19 (m, 10H), 3.23 (t, 2H), 4.30 (t, 2H), 5.00 (d, J=1.8 Hz, 1H), 5.51 (d, J=1.8 Hz, 1H), 6.57-7.25 (m, 10H); MS m/z 454 (M⁺)

### EXAMPLE 108

### Preparation of

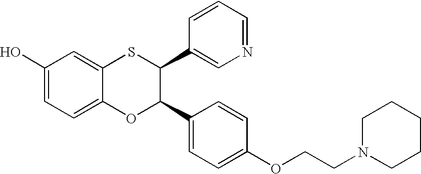

Step A: Reductive Cyclization

Following the procedure outlined in Example 44, 0.0792 g of the 3-pyridyl derivative prepared in Example 41 was converted to its corresponding benzoxathiin after stirring at ambient temperature for 5 h. The desired product was isolated from the reaction mixture after silica gel chromatography using 30% EtOAc/hexane as the eluant. ¹H 500 MHz NMR(CDCl₃) ppm(δ): 1.11 (d, 18H), 1.24 (m, 3H), 4.36 (d, J=2.1 Hz, 1H), 5.05 (s, 2H), 5.50 (d, J=1.6 Hz, 1H), 6.77-8.43 (m, 16H).

Step B: Desilylation

Following the procedure outlined in Example 105 (Step B), the dihydrobenzoxathiin generated in Step A above was desilylated to afford the desired product after silica gel chromatography (one elution with 50% EtOAc/hexane followed by a second elution with 30% EtOAc/hexane). ¹H 500 MHz NMR(CDCl₃) ppm(δ): 4.42 (d, J=2.1 Hz, 1H), 5.07 (s, 2H), 5.50 (d, J=1.6 Hz, 1H), 6.77-8.43 (m, 16H).

Step C: Mitsunobu Reaction

Following the procedure detailed in Example 105 (Step C) with the exception that the reaction was allowed to warm from 0° C. to ambient temperature over 4 h, the material prepared in the previous step was converted to the desired product after silica gel chromatography using 10% MeOH/CH₂Cl₂ as the eluant. ¹H 500 MHz NMR(CDCl₃) ppm(δ): 1.40-2.60 (m, 10H), 2.80 (t, 2H), 4.10 (t, 2H), 4.38 (d, J=1.8 Hz, 1H), 5.07 (s, 2H), 5.50 (d, J=1.8 Hz, 1H), 6.77-8.43 (m, 16H).

Step D: Debenzylation

Starting with the material prepared in Step C above, and utilizing the procedure outlined in Example 105 (Step D), the corresponding cis-3-pyridyl-dihydrobenzoxathiin adduct was prepared after silica gel chromatography with 10% MeOH/CH₂Cl₂ as the eluant. ¹H 500 MHz NMR(CDCl₃) ppm(δ): 1.40-2.60 (m, 10H), 2.80 (t, 2H), 4.10 (t, 2H), 4.36 (d, J=2.1 Hz, 1H), 5.45 (d, J=1.9 Hz, 1H), 6.59-8.43 (m, 11H); MS m/z 449 (M⁺).

### EXAMPLE 109

### Preparation of

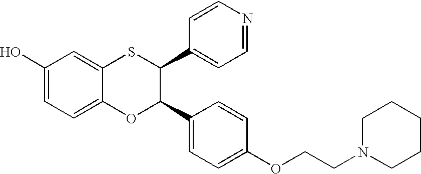

Step A: Reductive Cyclization

Following the procedure outlined in Example 44, 0.1871 g of the 4-pyridyl derivative prepared in Example 41 was converted to its corresponding dihydrobenzoxathiin after stirring at ambient temperature for 30 h. The desired product was isolated from the reaction mixture after silica gel chromatography using 30% EtOAc/hexane as the eluant. ¹H 500 MHz NMR(CDCl₃) ppm(δ): 1.11 (d, 18H), 1.24 (m, 3H), 4.32 (d, 1H), 5.08 (s, 2H), 5.50 (d, 1H), 6.60-8.39 (m, 16H).

Step B: Desilylation

Following the procedure outlined in Example 105 (Step B), the dihydrobenzoxathiin generated in Step A above was desilylated to afford the desired product after silica gel chromatography (one elution with 50% EtOAc/hexane followed by a second elution with 30% EtOAc/hexane). ¹H 500 MHz NMR(CDCl₃) ppm(δ): 4.33 (d, 1H), 5.07 (s, 2H), 5.46 (d, 1H), 6.63-8.37 (m, 16H).

Step C: Mitsunobu Reaction

Following the procedure detailed in Example 105 (Step C) with the exception that the reaction was allowed to warm from 0° C. to ambient temperature over 5 h, the material prepared in the previous step was converted to the desired product after silica gel chromatography (one elution with 10% MeOH/CH₂Cl₂ followed by a second elution with 20% EtOAc/CH₂Cl₂). ¹H 500 MHz NMR(CDCl₃) ppm(δ): 1.40-2.60 (m, 10H), 2.80 (t, 2H), 4.14 (t, 2H), 4.32 (d, J=3.0 Hz, 1H), 5.06 (s, 2H), 5.49 (d, J=2.1 Hz, 1H), 6.79-8.38 (m, 16H).

Step D: Debenzylation

Starting with the material prepared in Step C above, and utilizing the procedure outlined in Example 105 (Step D), the desired product was obtained as a 4:1 cis/trans mixture after silica gel chromatography (1× elution with 30% EtOAc/hexane followed by a second elution with 10% MeOH/CH₂Cl₂).

Cis isomer: ¹H 500 MHz NMR(CDCl₃) ppm(δ): 1.40-2.70 (m, 10H), 2.80 (t, 2H), 4.10 (t, 2H), 4.30 (d, J=2.0 Hz, 1H), 5.44 (d, J=1.8 Hz, 1H), 6.59-8.40 (m, 11H).

Trans isomer: 3H 500 MHz NMR(CDCl₃) ppm(δ): 1.40-2.70 (m, 10H), 2.80 (t, 2H), 4.15 (t, 2H), 4.38 (d, J=8.7 Hz, 1H), 4.92 (d, J=8.7 Hz, 1H), 6.59-8.46 (m, 11H); MS m/z 449 (M⁺).

### EXAMPLE 110

### Preparation of

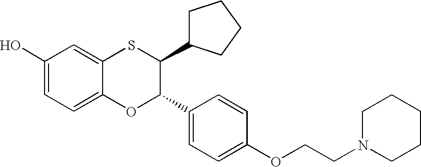

Step A: Reduction

To a stirred solution of 265.1 mg (0.449 mmole) of the cyclopentyl-thio-ketone generated in Example 41 in 3 mL of methanol-dichloromethane (1:1) at 0° C. to room temperature was added portion-wise sufficient sodium borohydride to complete the reduction. The reaction mixture was partitioned between ethyl acetate/2N HCl/ice/brine, and the organic phase was separated, washed with brine, dried over anhydrous sodium sulfate, filtered, and evaporated to provide crude cyclopentyl-thio-carbinols, which was used without further purification in the next step.

Step B: Cyclization

A mixture of 266 mg (0.449 mmole) of the crude product, prepared in Step A above, and 89 mg of amberlyst 15 in 3 mL of toluene was stirred at ambient temperature for two hours. The resin was removed by filtration and washed well with ethyl acetate. The filtrate was evaporated and the residue obtained was purified by silica gel chromatography using dichloromethane-hexanes (1:1) as eluant to provide the trans-dihydro-benzoxathiin derivative. ¹H 500 MHz NMR(CDCl₃) ppm(δ): 1.13 (d, 18H), 1.26-1.94 (m, 12H), 3.64 (dd, J=7.8 Hz, 5.5 Hz, 1H), 4.78 (d, J=7.8 Hz, 1H), 5.02 (s, 2H), 6.6-7.45 (m, 12H).

Step C: Desilylation

Following the procedure outlined in Step B of Example 105, 228.5 mg (0.397 mmole) of material prepared in the previous step was desilylated to give the corresponding phenol.

Step D: Mitsunobu Reaction

Following the procedure detailed in Step C of Example 105, the material prepared in the previous step was converted to the corresponding trans-cyclopentyl-dihydrobenzoxathiin adduct. ¹H 500 MHz NMR(CDCl₃) ppm(δ): 1.39-2.0 (m, 15H), 2.6 (m, 4H), 2.88 (m, 2H), 3.66 (dd, J=7.8 Hz, 5.5 Hz, 1H), 4.21 (m, 2H), 4.81 (t, J=7.8 Hz, 2H), 5.01 (s, 2H), 6.64-7.49 (m, 12H).

Step E: Debenzylation

Following the procedure detailed in Step D of Example 105, the material prepared in the previous step was converted to the corresponding trans-cyclopentyl-dihydrobenzoxathiin product. ¹H 500 MHz NMR(CDCl₃) ppm(δ): 1.29-2.0 (m, 15H), 2.6 (m, 4H), 2.88 (m, 2H), 3.67 (dd, J=8 Hz, 5 Hz, 1H), 4.18 (m, 2H), 4.77 (t, J=8 Hz, 2H), 6.5 (dd. J=2.7 Hz, 8.7 Hz, 1H), 6.65 (d, 2.7 Hz, 1H) 6.77 (d, J=8.7 Hz, 1H), 6.88 (d, J=7.5 Hz, 2H), and 7.27 (d, J=7.5 Hz, 2H).

### EXAMPLE 111

### General Preparation of

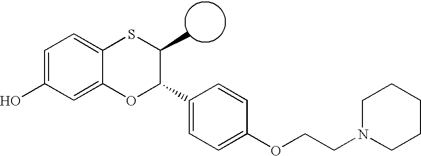

Steps A and B: Reduction and Cyclization

Utilizing the thio-ketones prepared in Example 39 and employing the procedures outlined above in Step A and B of Example 110, the following compounds were prepared:

Trans-cyclohexyl derivative: ¹H 500 MHz NMR(CDCl₃) ppm(δ): 1.14 (d, 18H), 0.98-1.8 (m, 14H), 3.37 (dd, J=2.5 Hz, 8.1 Hz, 1H), 5.01 (s, 2H), 5.05 (d, J=8.1 Hz, 1H), 6.6-7.44 (m, 12H).

Trans-cyclopentyl derivative: ¹H 500 MHz NMR(CDCl₃) ppm(δ): 1.14 (d, 18H), 1.28-1.9 (m, 12H), 4.53 (m, 1H), 4.93 (d, 1H), 5.01 (s, 2H), 6.6-7.43 (m, 12H).

Step C: Desiltylation

Utilizing the trans-dihydrobenzoxathiiins prepared in the previous step and employing the procedure outlined above in Step B of Example 105, the following compounds were prepared:

Trans-cyclohexyl phenol: ¹H 500 MHz NMR(CDCl₃) ppm(δ): 1.0-1.8 (m, 11H), 3.3 (m, 1H), 5.05 (s, 2H), 5.1 (d, 1H), 6.6-7.44 (m, 12H).

Trans-cyclopentyl phenol: ¹H 500 MHz NMR(CDCl₃) ppm(δ): 1.29-2.0 (m, 9H), 3.55 (dd, J=5.7 Hz, 7.6 Hz, 1H), 4.95 (d, J=7.6 Hz, 1H), 5.02 (s, 2H), 6.6-7.45 (m, 12H).

Step D: Mitsunobu Reaction:

Utilizing the trans-dihydrobenzoxathiiin phenols prepared in the previous step and employing the procedure outlined above in Step C of Example 105, the following compounds were prepared:

Trans-cyclohexyl adduct: ¹H 500 MHz NMR(CDCl₃) ppm(δ): 1.0-1.8 (m, 17H), 2.58 (m, 4H), 2.84 (m, 2H), 3.37 (m, 1H), 4.17 (t, J=6 Hz, 2H), 5.0 (s, 2H), 5.08 (d, J=7.8 Hz, 1H), 6.6-7.43 (m, 12H).

Trans-cyclopentyl adduct: ¹H 500 MHz NMR(CDCl₃) ppm(δ): 1.29-2.0 (m, 15H), 2.58 (m, 4H), 2.84 (m, 2H), 3.55 (m, 1H), 4.17 (m, 2H), 4.94 (d, J=7.3 Hz, 1H), 5.0 (s, 2H), 6.6-7.72 (m, 12H).

Step E: Debenzylation:

Utilizing the trans-dihydrobenzoxathiiin adducts prepared in the previous step and employing the procedure outlined above in Step D of Example 105, the following compounds were prepared:

Trans-cyclohexyl adduct: ¹H 500 MHz NMR(CDCl₃) ppm(δ): 1.0-1.8 (m, 17H), 2.58 (m, 4H), 2.86 (m, 2H), 3.33 (m, 1H), 4.16 (m, 2H), 5.08 (d, J=7.8 Hz, 1H), 6.4-7.23 (m, 7H).

Trans-cyclopentyl adduct: ¹H 500 MHz NMR(CDCl₃) ppm(δ): 1.29-2.0 (m, 15H), 2.68 (m, 4H), 2.94 (m, 2H), 3.51 (m, 1H), 4.2 (m, 2H), 4.95 (d, J=7.4 Hz, 1H), 6.45-7.31 (m, 7H).

### EXAMPLE 112

### Preparation of

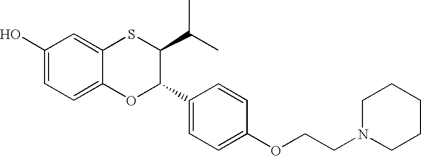

Step A: Silylation

To a stirred solution of the isopropyl-thio-ketone (0.0395 g, 0.097 mmol) generated in Example 42 in distilled THF (1 mL) at 0° C. was added 60% NaH in mineral oil (0.0183 g, 0.20 mmol) followed by TIPSCl (0.048 mL, 0.22 mmol). After 35 min., another equivalent of TIPSCl was added to drive the reaction to completion. The reaction was partitioned between EtOAc and ice/H₂O, and the organic layer was washed with brine, dried over Na₂SO₄, and concentrated in vacuo to afford the desired product. The crude material was used in the next step without further purification.

Step B: Reduction

To a solution of the crude product (0.097 mmol) prepared in Step A above in distilled THF (1 mL) was added a 1 M solution of super-hydride in THF (0.15 mL, 0.15 mmol) at 0° C. under N₂. The reaction mixture was stirred for 20 min. before partitioning between EtOAc and ice/H₂O. The organic layer was further washed with brine, dried over Na₂SO₄, and concentrated in vacuo to give the desired product. The crude material was used in the next step without further purification. ¹H 500 MHz NMR(CDCl₃) ppm(δ): 0.90-1.40 (m, 49H), 1.69 (m, 1H), 3.10 (dd, 1H), 4.60 (d, 1H), 5.05 (s, 2 H), 6.70-7.50 (m, 12H).

Step C: Desilylation

To a solution of the material (0.097 mmol) prepared in the previous step in distilled THF (1 mL) was added AcOH (0.018 mL, 0.32 mmol) at 0° C. under N₂ followed by the addition of a 1 M solution of TBAF in THF (0.29 mL, 0.29 mmol). After 15 min., the reaction was partitioned between EtOAc and ice/sat. NaHCO₃. The organic layer was washed with brine, dried over Na₂SO₄, and concentrated in vacuo. Purification by silica gel chromatography using 40% EtOAc/hexane as the eluant afforded the desired product as a yellow foam. ¹H 500 MHz NMR(CDCl₃) ppm(δ): 0.92 (d, 3H), 0.98 (d, 3H), 1.59 (m, 1H), 2.86 (dd, 1H), 4.62 (d, 1H), 5.02 (q, 2 H), 6.77-7.45 (m, 12H).

Step D: Cyclization

Following the procedure outlined in Example 110 (Step B), the material (0.0366 g, 0.089 mmol) generated in the previous step was converted to its corresponding trans-dihydrobenzoxathiin after stirring for 5 h 15 min. at ambient temperature.

Purification by silica gel chromatography using 30% EtOAc/hexane as the eluant afforded the desired product as a white solid. ¹H 500 MHz NMR(CDCl₃) ppm(δ): 0.98 (d, 3H), 1.03 (d, 3H), 1.78 (m, 1H), 3.57 (dd, J=3.7 Hz, J=8.5 Hz, 1H), 4.82 (d, J=8.4 Hz, 1H), 5.02 (s, 2 H), 6.63-7.46 (m, 12H).

Step E: Mitsunobu Reaction

Following the procedure detailed in Example 105 (Step C), the material (0.0266 g, 0.068 mmol) generated in the previous step was converted to its corresponding trans-isopropyl-dihydrobenzoxathiin adduct after warming from 0° C. to ambient temperature over 4 h 20 min. Purification by silica gel chromatography (one elution with 10% MeOH/CH₂Cl₂ followed by a second elution with 30% EtOAc/hexane) afforded the desired product as a white solid. ¹H 500 MHz NMR(CDCl₃) ppm(δ): 0.98 (d, 3H), 1.02 (d, 3H), 1.29-1.67 (m, 6H), 1.78 (m, 1H), 2.58 (m, 4H), 2.85 (t, 2H), 3.57 (dd, J=3.7 Hz, J=8.5 Hz, 1H), 4.18 (t, 2H), 4.83 (d, J=8.4 Hz, 1H), 5.02 (s, 2 H), 6.63-7.46 (m, 12H).

Step F: Debenzylation

Following the procedure detailed in Example 105 (Step D), the material (0.0395 g, 0.068 mmol) generated in the previous step was converted to its corresponding trans-isopropyl-dihydrobenzoxathiin product. Purification was accomplished by silica gel chromatography using 10% MeOH/CH₂Cl₂ as the eluant. ¹H 500 MHz NMR(CDCl₃) ppm(δ): 0.98 (d, 3H), 1.02 (d, 3H), 1.29-1.67 (m, 6H), 1.78 (m, 1H), 2.58 (m, 4H), 2.85 (t, 2H), 3.57 (dd, J=3.7 Hz, J=8.5 Hz, 1H), 4.18 (t, 2H), 4.83 (d, J=8.4 Hz, 1H), 6.48-7.29 (m, 7H); MS m/z 414 (M⁺).

### EXAMPLE 113

### Preparation of

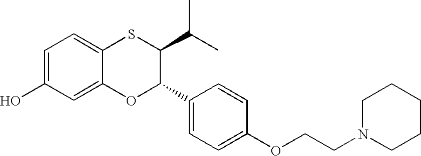

Step A: Silylation

Following the procedure outlined in Example 112 (Step A), the isopropyl-thio-ketone (0.6314 g, 1.5 mmol) generated in Example 40 was silylated. Purification by silica gel chromatography using 30% EtOAc/hexane as the eluant afforded the desired product as a yellow oil. ¹H 500 MHz NMR(CDCl₃) ppm(δ): 0.98-1.30 (m, 49H), 2.35 (m, 1H), 4.38 (d, 1H), 4.99 (q, 2H), 6.33-7.79 (m, 12H).

Step B: Reduction

Following the procedure outlined in Example 112 (Step B), the material (0.8009 g, 1.1 mmol) isolated in Step A above was reduced to its corresponding alcohol and used without further purification in the next step. ¹H 500 MHz NMR(CDCl₃) ppm(δ): 0.98-1.30 (m, 49H), 1.90 (m, 1H), 2.92 (dd, 1H), 4.59 (d, 1H), 5.05 (q, 2 H), 6.47-7.43 (m, 12H).

Step C: Desilylation

Following the procedure outlined in Example 112 (Step C), the material (0.022 mmol) isolated in Step B above was deprotected to afford the desired product which was used in the next step without purification.

Step D: Cyclization

Following the procedure outlined in Example 110 (Step B), the material generated in the previous step was converted to its corresponding trans-dihydrobenzoxathiin after stirring for 22 h at ambient temperature. Purification by silica gel chromatography using 30% EtOAc/hexane as the eluant afforded the desired product as a colorless oil. ¹H 500 MHz NMR(CDCl₃) ppm(δ): 0.98 (d, 3H), 1.03 (d, 3H), 1.79 (m, 1H), 3.45 (dd, 1H), 4.98 (d, 1H), 5.02 (s, 2 H), 6.59-7.46 (m, 12H); MS m/z 393 (M⁺).

Step E: Mitsunobu Reaction

Following the procedure detailed in Example 105 (Step C), the material (0.008 g, 0.020 mmol) generated in the previous step was converted to its corresponding trans-isopropyl-dihydrobenzoxathiin adduct after warming from 0° C. to ambient temperature over 6 h. Purification by silica gel chromatography using 10% MeOH/CH₂Cl₂ as the eluant afforded the desired product as a pale yellow oil. ¹H 500 MHz NMR(CDCl₃) ppm(δ): 0.98 (d, 3H), 1.02 (d, 3H), 1.29-1.67 (m, 6H), 1.79 (m, 1H), 2.58 (m, 4H), 2.81 (t, 2H), 3.50 (dd, J=3.8 Hz, J=8.3 Hz, 1H), 4.18 (t, 2H), 4.97 (d, J=8.2 Hz, 1H), 5.01 (s, 2 H), 6.59-7.46 (m, 12H).

Step F: Debenzylation

Following the procedure detailed in Example 105 (Step D), the material (0.0085 g, 0.017 mmol) generated in the previous step was converted to its corresponding trans-isopropyl-dihydrobenzoxathiin product. Purification was accomplished by silica gel chromatography using 10% MeOH/CH₂Cl₂ as the eluant. ¹H 500 MHz NMR(CDCl₃) ppm(δ): 0.98 (d, 3H), 1.02 (d, 3H), 1.49-1.70 (m, 6H), 1.75 (m, 1H), 2.61 (m, 4H), 2.85 (t, 2H), 3.41 (dd, J=3.8 Hz, J=8.3 Hz, 1H), 4.18 (t, 2H), 4.96 (d, J=8.2 Hz, 1H), 6.43-7.26 (m, 7H); MS m/z 414 (M⁺).

Assay Methods

The utility of the compounds of the instant invention can be readily determined by methods well known to one of ordinary skill in the art. These methods may include, but are not limited to, the methods described in detail below.

Estrogen Receptor Binding Assay

The estrogen receptor ligand binding assays are designed as scintillation proximity assays employing the use of tritiated estradiol and recombinant expressed estrogen receptors. The full length recombinant human ER-α and ER-β proteins are produced in a bacculoviral expression system. ER-α or ER-β extracts are diluted 1:400 in phosphate buffered saline containing 6 mM α-monothiolglycerol. 200 μL aliquots of the diluted receptor preparation are added to each well of a 96-well Flashplate. Plates are covered with Saran Wrap and incubated at 4° C. overnight.

The following morning, a 20 ul aliquot of phosphate buffered saline containing 10% bovine serum albumin is added to each well of the 96 well plate and allowed to incubate at 4° C. for 2 hours. Then the plates are washed with 200 ul of buffer containing 20 mM Tris (pH 7.2), 1 mM EDTA, 10% Glycerol, 50 mM KCl, and 6 mM α-monothiolglycerol. To set up the assay in these receptor coated plates, add 178 ul of the same buffer to each well of the 96 well plate. Then add 20 ul of a 10 nM solution of ³H-estradiol to each well of the plate.

Test compounds are evaluated over a range of concentrations from 0.01 nM to 1000 nM. The test compound stock solutions should be made in 100% DMSO at 100× the final concentration desired for testing in the assay. The amount of DMSO in the test wells of the 96 well plate should not exceed 1%. The final addition to the assay plate is a 2 ul aliquot of the test compound which has been made up in 100% DMSO. Seal the plates and allow them to equilibrate at room temperature for 3 hours. Count the plates in a scintillation counter equipped for counting 96 well plates.

Ovariectomized Rat Assay

In the ovariectomized (OVX) Rat Assay, estrogen-deficiency is used to induce cancellous osteopenia (e.g. low bone mineral density [BMD; mg/cm²]), associated with accelerated bone resorption and formation. Both the BMD and bone resorption/formation outcomes are used to model the changes in bone that occur as women pass through menopause. The OVX Rat Assay is the principal in vivo assay used by all major academic and industrial laboratories studying the efficacy of new chemical entities in preventing estrogen-deficiency bone loss.

Sprague-Dawley female rats aged 6-8 months are OVXd and, within 24 hours, started on treatment for 42 days with vehicle or multiple doses of test compound. Untreated sham-OVX and alendronate-treated (0.003 mg/kg s.c., q.d.) or 17-β-estradiol-treated (0.004 mg/kg s.c., q.d.) groups are included as positive controls. Test compounds may be administered orally, subcutaneously, or by infusion through subcutaneously-implanted minipump. Before necropsy, in vivo dual labeling with calcein (8 mg/kg by subcutaneous injection), a bone seeking fluorochrome, is completed. At necropsy, blood, femurs, a vertebral body segment, and the uterus, are obtained.

The routine endpoints for the OVX Rat Assay include assessments of bone mass, bone resorption, and bone formation. For bone mass, the endpoint is BMD of the distal femoral metaphysis, a region that contains about 20% cancellous bone. The vertebral segment, a region with ˜25% cancellous bone may also be used for BMD determination. The BMD measurement is made by dual energy x-ray absorptiometry (DXA, Hologic 4500A; Waltham, Mass.). For bone resorption, the endpoint is urinary deoxypyridinoline crosslinks, a bone collagen breakdown product (uDPD; expressed as nM DPD/nM creatinine). This measurement is made with a commercially available kit (Pyrilinks; Metra Biosystems, Mountain View, Calif.). For bone formation, the endpoints are mineralizing surface and mineral apposition rate, histomorphometric measures of osteoblast number and activity. This measurement is done on 5 μm sections of the non-decalcified proximal tibial metaphysis, using a semi-automated system (Bioquant; R&M Biometrics; Nashville, Tenn.). Similar endpoints and measuring techniques for each endpoint are commonly used in postmenopausal women.

Rat Cholesterol Lowering Assay

Sprague-Dawley rats (5 per group) weighing about 250 g were subcutaneously dosed with compounds of the present invention dissolved in propylene glycol for 4 days. A group of 5 rats were dosed with vehicle only. On the fifth day, rats were euthanized with carbon dioxide and their blood samples were obtained. Plasma levels of cholesterol were assayed from these samples with commercially available cholesterol determination kits from Sigma.

MCF-7 Estrogen Dependent Proliferation Assay

MCF-7 cells (ATCC #HTUB-22) are human mammary gland adenocarcinoma cells that require estrogen for growth. The growth media (GM) for the MCF-7 cells is Minimum Essential Media (without phenol red) supplemented with fetal bovine serum(FBS) to 10%. The FBS serves as the sole source of estrogen and this GM supports the full growth of the cells and is used for the routine growth of the cell cultures. When MCF-7 cells are placed in a media in which 10% Charcoal-Dextran treated fetal bovine serum (CD-FBS) is substituted for FBS, the cells will cease to divide but will remain viable. The CD-FBS does not contain detectable levels of estrogen and the media containing this sera is referred to as Estrogen Depleted Media (EDM). The addition of estradiol to EDM stimulates the growth of the MCF-7 cells in a dose dependent manner with an EC₅₀ of 2 μM.

Growing MCF-7 cells are washed several times with EDM and the cultures then maintained in EDM for a minimum of 6 days in order to deplete the cells of endogenous estrogen. On day 0 (at the start of the assay), these estrogen depleted cells are plated into 96-well cell culture plates at a density of 1000 cells/well in EDM in a volume of 180 ul/well. On day 1 test compounds are diluted in a 10-fold dilution series in EDM and 20 ul of these dilutions added to the 180 ul of media in the appropriate well of the cell plate resulting in a further 1:10 dilution of the test compounds. On days 4 and 7 of the assay, the culture supernatant is aspirated and replaced with fresh EDM and test compound dilutions as above. The assay is terminated at day 8-10 when the appropriate controls reach 80-90% confluency. At this point, the culture supernatants are aspirated, the cells washed 2× with PBS, the wash solution aspirated and the protein content of each well determined. Each drug dilution is evaluated on a minimum of 5 wells and the range of dilution of the test compounds in the assay is 0.001 nM to 1000 nM. The assay in the above format is employed to determine the estradiol agonist potential of a test compound.

In order to evaluate the antagonist activity of a test compound, the MCF-7 cells are maintained in EDM for a minimum of 6 days. Then on day 0 (at the start of the assay), these estrogen depleted cells are plated into 96-well cell culture plates at a density of 1000 cells/well in EDM in a volume of 180 ul/well. On day 1 the test compounds in fresh media containing 3 pM estradiol are applied to the cells. On days 4 and 7 of the assay, the culture supernatant is aspirated and replaced with fresh EDM containing 3 pM estradiol and the test compound. The assay is terminated at day 8-10 when the appropriate controls reach 80-90% confluency and the protein content of each well is determined as above.

Rat Endometriosis Model

Animals:

Species: Rattus norvegicus

Strain: Sprague-Dawley CD

Supplier: Charles River Laboratories, Raleigh, N.C.

Sex: Female Weight: 200- 240 gram

Rats are single-housed in polycarbonate cages and are provided Teklad Global Diet 2016 (Madison, Wis.) and bottled reverse osmosis purified H₂O ad libitum. They are maintained on a 12/12 light/dark cycle.

Rats are anesthetized with Telazol™ (20 mg/kg, ip) and oxymorphone (0.2 mg/kg sc) and positioned dorsoventrally on a sterile drape. Body temperature is maintained using a underlying circulating water blanket. The surgical sites are shaved with clippers and cleaned using three cycles of betadine/isopropyl alcohol or Duraprep® (3M). The incisional area is covered with a sterile drape.

Using aseptic technique, a 5 cm midline lower abdominal incision is made through the skin, subcutaneous and muscle layers. A bilateral ovariectomy is performed. The left uterine blood vessels are ligated and a 7 mm segment of the left uterine horn is excised. The uterus is closed with 4-0 gut suture. The myometrium is aseptically separated from the endometrium and trimmed to 5×5 mm. The trimmed section of the endometrium is transplanted to the ventral peritoneal wall with the epithelial lining of the segment opposed to the peritoneal wall. The explanted endometrial tissue is sutured at its four corners to the body wall using sterile 6-0 silk. The abdominal muscular layer is closed using sterile 4-0 chromic gut. The skin incision is closed using sterile stainless surgical clips. A sterile 90-day sustained release estrogen pellet (Innovative Research of America, 0.72 ng/pellet; circulating estrogen equivalent of 200-250 pg/mL) is implanted subcutaneously in the dorsal lateral scapular area. A sterile implantable programmable temperature transponder (IPTT) (BMDS, Seaford, Del.) is injected subcutaneously in the dorsoscapular region. The rats are observed until fully ambulatory, and allowed to recover from surgery undisturbed for 3 weeks.

Three weeks after transplantation of the endometrial tissue, the animals undergo a repeat laparotomy using aseptic surgical site preparation and technique. The explant is evaluated for graft acceptance, and the area is measured with calipers and recorded. The animals with rejected grafts are removed from the study. Animals are sorted to create similar average explant volume per group.

Drug or vehicle (control) treatment is initiated one day after the second laparotomy and continued for 14 days. Body temperature is recorded every other day at 10:00 am using the BMDS scanner.

At the end of the 14 day treatment period, the animals are euthanized by CO₂ overdose. Blood is collected by cardiocentesis for circulating estrogen levels. The abdomen is opened, the explant is examined, measured, excised, and wet weight is recorded. The right uterine horn is excised, and wet and dry weights are recorded.

Pharmaceutical Composition

As a specific embodiment of this invention, 25 mg of the compound from Example 71, is formulated with sufficient finely divided lactose to provide a total amount of 580 to 590 mg to fill a size 0, hard-gelatin capsule.

What is claimed is:

1. A process for preparing a compound of formula I 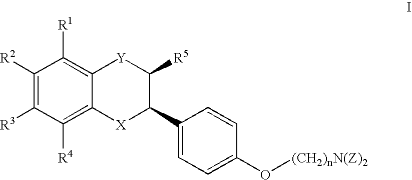
wherein R¹ is H, F, or Cl;
R² is H or OR⁶;
R³ is H or OR⁶;
R⁴ is H or CH₃;
R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SONZ₂), and sulfonyl (—SO₂C₁₋₅ alkyl);
R⁶ is H, benzyl, methyl, methoxymethyl, or trisopropylsilyl, with the proviso that when OR⁶ exists elsewhere, it is chemically differentiable;
X and Y are each independently selected from the group consisting of oxygen and sulfur;
Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Each Q is independently selected from the group consisting of C₁₋₅ alkyl, CF₃, —OR⁶, halogen, amino, C₁₋₅ alkylthio, thiocyanato, cyano, —CO₂H, —COOC₁₅ alkyl, —COC₁₋₅ alkyl, and —SO₂C₁₋₅ alkyl;
Each n is independently an integer from one to five; n is an integer from one to five; and the stereoisomer is cis;
or a pharmaceutically acceptable salt thereof,
comprising alkylating the substituted phenol of formula VI, with a reagent, HO(CH₂)ₙN(Z)₂, to give a compound of formula I.

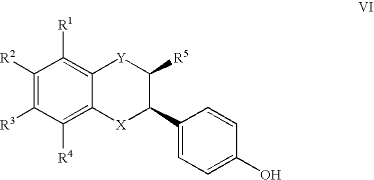

2. The process according to claim 1 further comprising the step of removing the protecting group R⁶ from V to yield the substituted phenol of formula VI.

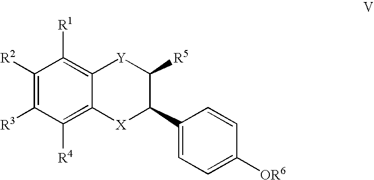

3. The process of claim 2 further comprising the step of cyclizing IV under acidic conditions in the presence of a reducing agent, to provide the cis compound of formula V.

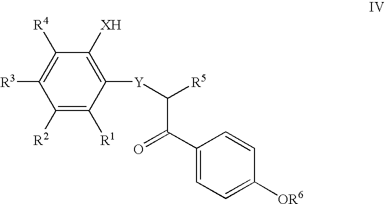

4. The process of claim 3 further comprising the step of reacting a compound of formula II with a compound of formula III under basic conditions 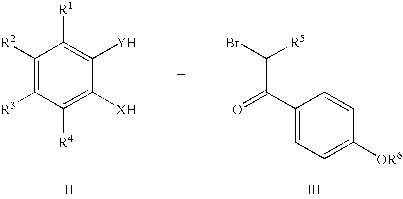
to form a compound of formula IV.

5. The process according to claim 4 wherein Y is S and X is O.

6. The process according to claim 5 for preparing a compound of formula IB 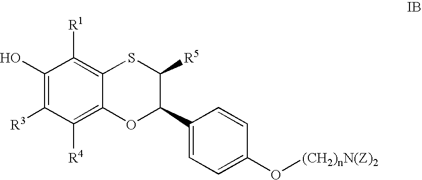
wherein R¹ is H, F, or Cl;
R³ is H or OR⁶;
R⁴ is H or CH₃;
R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SONZ₂), and sulfonyl (—SO₂C₁₋₅ alkyl);
R⁶ is H, benzyl, methyl, methoxymethyl, or trisopropylsilyl, with the proviso that when OR⁶ exists elsewhere, it is chemically differentiable;
Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl.
Each n is independently an integer from one to five;
and the stereoisomer is cis;
or a pharmaceutically acceptable salt thereof,
comprising the steps of
a) reacting a compound of formula IIB with a compound of formula IIIB under basic conditions 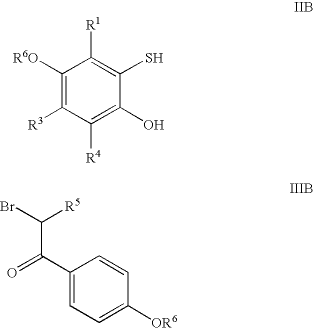
to form a compound of formula IVB 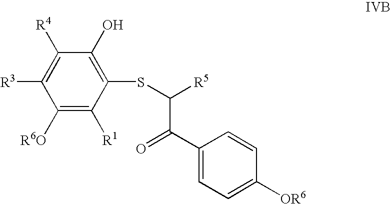
b) cyclizing the compound of formula IVB, of step a, under acidic conditions in the presence of a reducing agent to provide the cis compound of formula VB 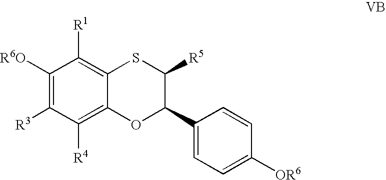
c) selectively removing the protecting group R⁶ to yield the substituted phenol of formula VIB 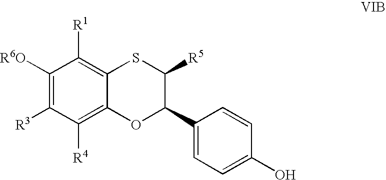
d) alkylating the substituted phenol of formula VIB, from step c, with a reagent, HO(CH₂)ₙN(Z)₂ to give a compound of formula VIIB 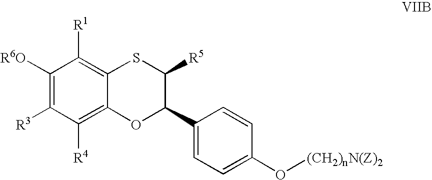
e) removing the protecting group R⁶ of VIIB, from step d, to afford a compound of formula I.

7. The process according to claim 6 for preparing a compound of formula IC 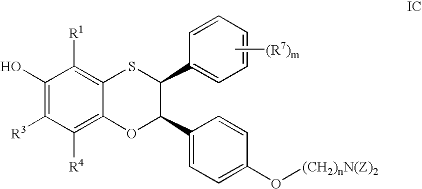
wherein R¹ is H, F, or Cl;
R³ is H or OR⁶;
R⁴ is H or CH₃;
R⁶ is H, benzyl, methyl, methoxymethyl, or trisopropylsilyl, with the proviso that when OR⁶ exists elsewhere, it is chemically differentiable;
R⁷ is selected from the group selected from the group consisting of hydrogen, C₁₋₅ alkyl, halogen, trifluoromethyl, and —OR⁶;
Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl.
Each n is independently an integer from one to five;
n is an integer from one to five;
m is an integer from one to four;
and the stereoisomer is cis;
or a pharmaceutically acceptable salt thereof,
comprising the steps of
a) reacting a compound of formula IIC with a compound of formula IIIC under basic conditions 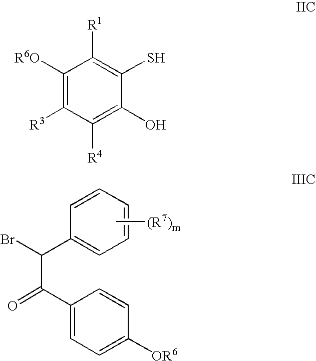
to form a compound of formula IVC 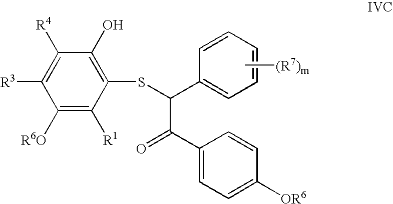
b) cyclizing IVC, of step a, under acidic conditions in the presence of a reducing agent to provide the cis compound of formula VC 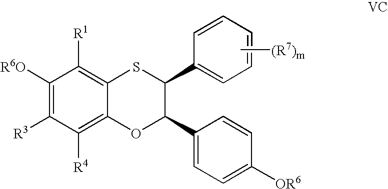
c) selectively removing the protecting group R⁶ to yield the substituted phenol of formula VIC 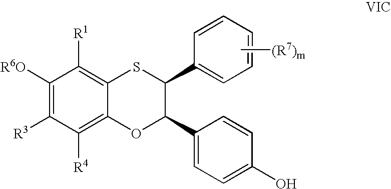
d) alkylating the substituted phenol VIC, from step c, with a reagent, HO(CH₂)ₙN(Z)₂ to give a compound of formula VIIC 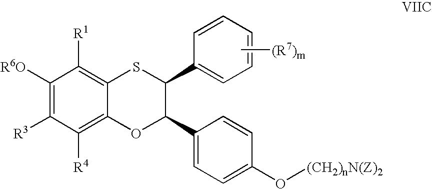
e) removing the protecting group R⁶ of VIIC, from step d, to afford a compound of formula IC.

8. A process for preparing a compound of formula ID 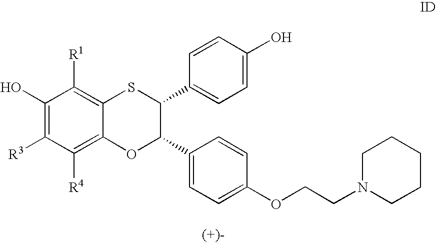
wherein R¹ is H, F, or Cl;
R³ is H or OR⁶;
R⁴ is H or CH₃;
R⁶ is H, benzyl, methyl, methoxymethyl, or trisopropylsilyl, with the proviso that when OR⁶ exists elsewhere, it is chemically differentiable;
and the stereoisomer is cis;
and the optical isomer is dextrorotatory (+), having the absolute configuration: (2S, 3R);
or a pharmaceutically acceptable salt thereof,
comprising the steps of
a) reacting a compound of formula with a compound of formula IID with a compound of formula IIID under basic conditions 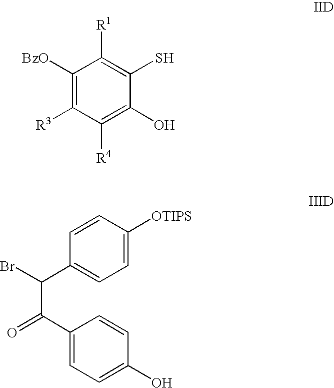
to form a compound of formula IVD 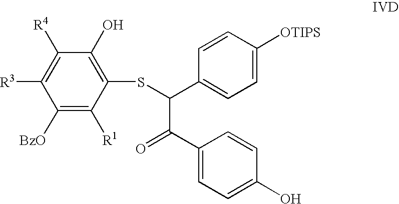
b) cyclizing IVD, of step a, under acidic conditions in the presence of a reducing agent to provide the racemic, cis compound of formula VD 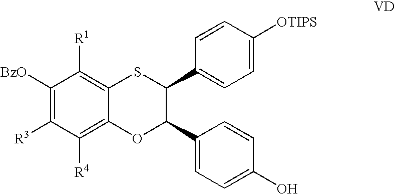
c) performing a chiral chromatography with VD, from step b, to resolve the enantiomeric forms to provide the dextrorotatory (+) isomer VID; 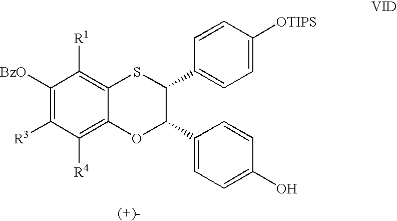
d) alkylating the dextrorotatory (+) isomer VID, from step c, with 1-piperidineethanol to give a compound of formula VIID 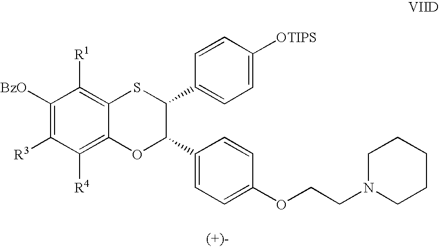
e) removing either protecting group from VIID, from step d, to afford either a compound of formula VIIID or a compound of formula IXD 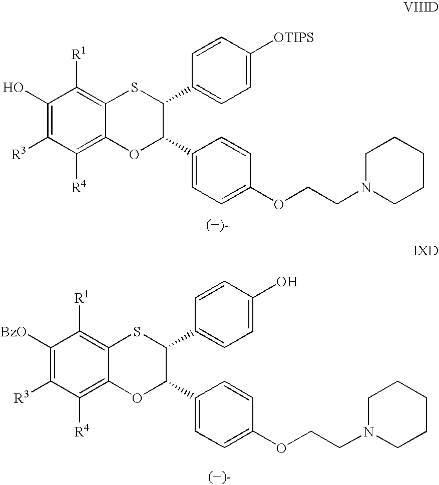
f) removing the remaining protecting group from either VIIID or IXD, from step e, to give a compound of formula (+)-ID.

9. A process for preparing a compound of formula IE 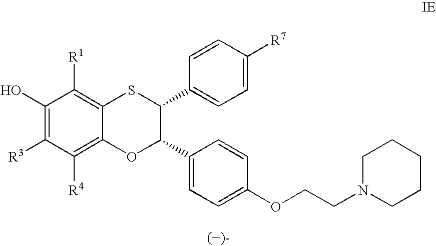
wherein
R¹ is selected from the group consisting of H, F, or Cl;
R³ and R⁴ are each H;
R⁷ is selected from the group consisting of H or OH;
the stereoisomer is cis, and the optical isomer is dextrorotatory (+), having the absolute configuration (2S, 3R);
or a pharmaceutically acceptable salt thereof
comprising the steps of
a) reacting a compound of formula IIE with a compound of formula IIIE under basic conditions 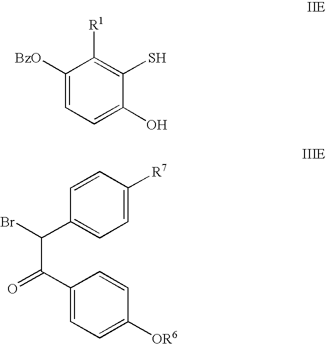
to form a compound of formula IVE 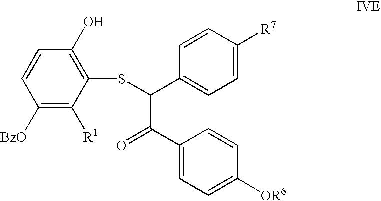
b) cyclizing IVE, of step a, under acidic conditions in the presence of a reducing agent to provide the racemic, cis compound of formula VE 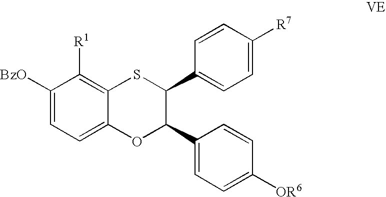
c) selectively removing the protecting group of VE, from step b, to yield the substituted phenol of formula VIE 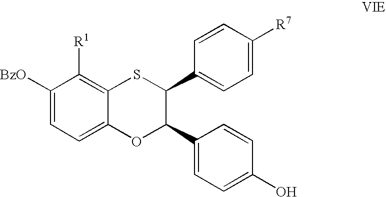
d) alkylating the substituted phenol of formula VIE, from step c, with 1-piperidineethanol to give a compound of formula VIIE 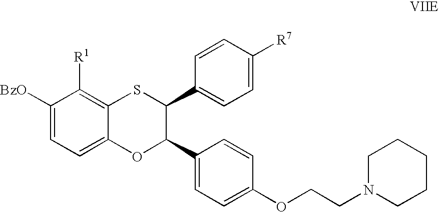
e) removing either protecting group from VIIE to afford either a compound of formula VIIIE or a compound of formula IXE 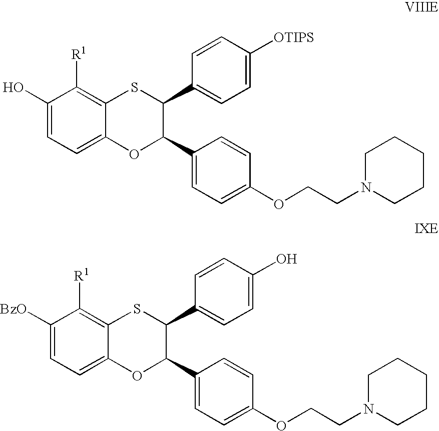
f) removing the remaining protecting group from either VIIE or IXE, from step e, to provide racemic I.
g) performing a resolution of the enantiomeric forms of I to provide the dextrorotatory (+) isomer IE, having the (2S, 3R) absolute configuration.

10. A compound of the formula: 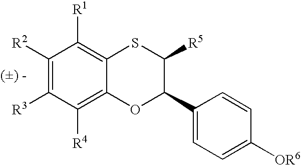
wherein R¹ is H, F, or Cl;
R² is H or OR⁶;
R³ is H or OR⁶;
R⁴ is H or CH₃;
R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SONZ₂), and sulfonyl (—SO₂C₁₋₅ alkyl);
R⁶ is H, benzyl, methyl, methoxymethyl, or trisopropylsilyl, with the proviso that when OR⁶ exists elsewhere, it is chemically differentiable;
Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl.

11. The compound of claim 10 of the formula: 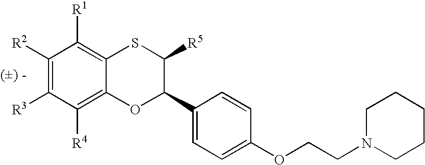
wherein R¹ is H, F, or Cl;
R² is H or OR⁶;
R³ is H or OR⁶;
R⁴ is H or CH₃;
R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, —OH, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SONZ₂), and sulfonyl (—SO₂C₁₋₅ alkyl);
R⁶ is benzyl, methyl, methoxymethyl, or trisopropylsilyl, with the proviso that when OR⁶ exists elsewhere, it is chemically differentiable;

12. The compound of claim 10 of the formula: 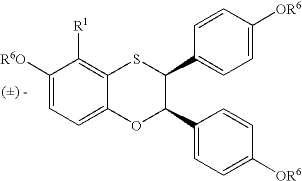
wherein R¹ is H, F, or Cl;
R⁶ is H, benzyl, methyl, methoxymethyl, or trisopropylsilyl, with the proviso that all existing R⁶ groups are chemically differentiable.

13. The compound of claim 12 of the formula: 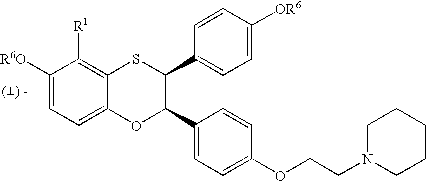
wherein R¹ is H, F, or Cl;
R⁶ is H, benzyl, methyl, methoxymethyl, or trisopropylsilyl, with the proviso that all existing R⁶ groups are chemically differentiable.

14. A compound of the formula: 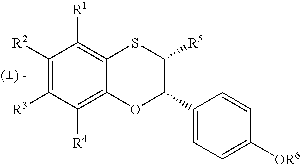
wherein R¹ is H, F, or Cl;
R² is H or OR⁶;
R³ is H or OR⁶;
R⁴ is H or CH₃;
R⁵ is C₁₋₅ alkyl, C₃₋₈ cycloalkyl, C₃₋₈ cycloalkenyl, phenyl, heteroaryl, or heterocyclical groups wherein said groups can be optionally substituted with C₁₋₅ alkyl, C₃₋₈ cycloalkyl, CF₃, phenyl, heteroaryl, heterocyclical, —OR⁶, halogen, amino, C₁₋₅ alkylthio, thiocyanato, cyano, carboxyl (—CO₂H), carboalkoxyl (—COOC₁₋₅ alkyl), carbonyl (—COC₁₋₅ alkyl, carboxamido (—CONZ₂), sulfonamido (—SONZ₂), and sulfonyl (—SO₂C₁₋₅ alkyl);
R⁶ is H, benzyl, methyl, methoxymethyl, or trisopropylsilyl, with the proviso that when OR⁶ exists elsewhere, it is chemically differentiable;
Each Z is independently selected from the group consisting of hydrogen, C₁₋₅ alkyl, trifluoromethyl, wherein said alkyl group can be optionally substituted with C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Or both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, —CONQ₂, —SO₂NQ₂, and —SO₂C₁₋₅ alkyl;
Each Q is independently selected from the group consisting of C₁₋₅ alkyl, CF₃, —OR⁶, halogen, amino, C₁₋₅ alkylthio, thiocyanato, cyano, —CO₂H, —COOC₁₋₅ alkyl, —COC₁₋₅ alkyl, and —SO₂C₁₋₅ alkyl.

15. The compound of claim 14 of the formula: 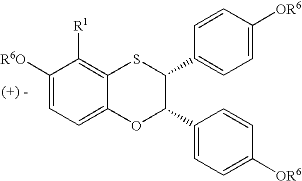
wherein R¹ is H, F, or Cl;
R⁶ is H, benzyl, methyl, methoxymethyl, or trisopropylsilyl, with the proviso that all existing R⁶ groups are chemically differentiable.

16. The compound of claim 15 of the formula: 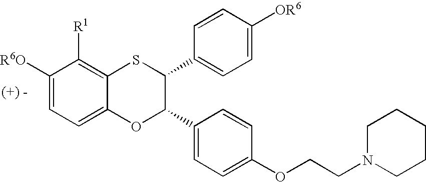
wherein R¹ is H, F, or Cl;
R⁶ is H, benzyl, methyl, methoxymethyl, or trisopropylsilyl, with the proviso that all existing R⁶ groups are chemically differentiable.
